(19) United States
(12) Patent Application Publication
(10) Pub. No.: US 2006/0122660 A1
(43) Pub. Date: Jun. 8, 2006

(54) Method and system for modulating sacral nerves and/or its branches in a patient to provide therapy for urological disorders and/or fecal incontinence, using rectangular and/or complex electrical pulses

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/320,434

(22) Filed: Dec. 28, 2005

Related U.S. Application Data
(63) Continuation-in-part of application No. 10/195,961, filed on Jul. 16, 2002.
Application No. 10/195,961 is a continuation of application No. 09/752,083, filed on Dec. 29, 2000, now Pat. No. 6,505,074.
Application No. 09/752,083 is a continuation of application No. 09/178,060, filed on Oct. 26, 1998, now Pat. No. 6,205,359.

(51) Int. Cl. A61N 1/08

(52) U.S. Cl. 607/40

(57) ABSTRACT

A method and system for providing pulsed electrical stimulation to sacral nerves and/or its branches, to provide therapy for urinary/fecal incontinence and other urological disorders. The stimulation system comprising implanted and external components. The pulsed electrical stimulation may be provided using a system which is one from a group comprising: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with an external magnet; d) a programmable implantable pulse generator; e) a combination implantable device comprising both a stimulus-receiver and a programmable IPG; and f) an implantable pulse generator (IPG) comprising a rechargeable battery. In one embodiment, the external components such as the programmer or external stimulator may comprise telemetry means for interrogation or programming of the implanted device from a remote location, over a wide area network.

This is a Continuation of application Ser. No. 10/195,961 which is a Continuation of application Ser. No. 09/752,083 (now U.S. Pat. No. 6,505,074) which is a Continuation-in Part of application Ser. No. 09/178,060 now U.S. Pat. No. 6,205,359 having a filing date of Oct. 26, 1998. Priority is claimed from these applications, and the prior applications being incorporated herein by reference.

### FIELD OF INVENTION

The present invention relates to electrical neuromodulation therapy for medical disorders, more specifically pulsed electrical neuromodulation therapy for urological disorders and/or fecal incontinence utilizing rectangular and/or complex electrical pulses.

### BACKGROUND

Biomedical and clinical research has shown utility of electrical nerve stimulation (neuromodulation) of sacral nerves or branches for urinary and fecal incontinence, and a broad group of urological disorders. This invention is directed to method and system for providing pulsed electrical stimulation/blocking therapy for urological disorders, and fecal incontinence. The urological disorders comprise urinary incontinence, overflow incontinence, stress incontinence, idiopathic chronic urinary retention, interstitial cystitis, neuro-urological disorder, vesico-urethral dysfunctions, bladder inflammation, bladder pain, pelvic pain, constipation, and genito-urinary disorders such as prostatitis, prostatalgia, and prostatodynia.

Pulse generator system to provide therapy for urinary incontinence and urological disorders are known in the art. But, pulse generator systems can be designed in different ways, and a particular type may be more suitable for an individual patient. For example, for patients requiring high stimulation outputs, an external stimulator which works in conjunction with an implanted stimulus-receiver would be appropriate, since a fully implantable system would have a short service life for such a patient. For patients requiring low stimulation outputs, an implantable system may be appropriate because of its convenience, and for patient compliance. This Application discloses six distinct types of pulse generator systems that can be used to provide therapy for urinary incontinence and/or fecal incontinence.

The seven types of systems disclosed in this Application to provide pulsed electrical stimulation to a patient, to provide therapy for urinary/fecal incontinence, and other urological disorders, are:

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a programmable implantable pulse generator (IPG);

e) a combination implantable device comprising both a stimulus-receiver and a programmable IPG; and

f) an IPG comprising a rechargeable battery.

In one aspect, a patient may be implanted with more than one type of pulse generator system over time, utilizing the same implanted lead. For example, a patient may be initially implanted with an implanted stimulus-receiver and the stimulation performed with an external stimulator, since an external stimulator can be adjusted by the patient, within the limits prescribed by the physician. This simple and inexpensive system can be used to evaluate a patient's response to neuromodulation therapy. If the patient responds well and neuromodulation therapy is to be continued, at a future time, the implanted stimulus-receiver can be exchanged with an implanted pulse generator (IPG), using the same lead.

In another example, a patient implanted with an implanted pulse generator (I PG) finds that the stimulation thresholds have increased, or the patient does better with high outputs, such that the battery is depleting prematurely. In such a patient, at replacement an IPG comprising rechargeable battery may be used, or an implantable stimulus-receiver may be implanted, and an external pulse generator may be used. Again, without replacing the original lead.

The external components of these systems may be networked over a wide area network, as disclosed in a co-pending application. These external components are either the external pulse generator, or the programmer for an implanted pulse generator.

In one aspect, since the pulse generator system and the patient can be monitored and remotely controlled, the appropriate therapy for each patient can be “customized” without the patient having to visit the clinic, for each adjustment or programming of the device.

With reference to prior art, U.S. Pat. No. 5,562,717 (Tippey et al.) teaches an external system comprising a portable electrical stimulator which can be coupled to one or more electrodes for applying electrical stimulation signals to a patient. The signal generator being responsive to the instruction storage or programming device.

U.S. Pat. No. 6,393,323 B1 (Sawan et al.) teaches a selective stimulation system which is composed of an internal stimulator implanted in the patient and operated with an external hand-held controller. The system being used to prevent bladder hyeperreflexia combined with a voiding signal generator generating a voiding signal for voiding the bladder.

U.S. Patent Applications 2004/0193228 (Gerber), 2005/0033372 (Gerber), 2005/0033374 (Gerber), and 2005/0010259 (Gerber) are generally directed to applying electrical stimulation signals, and/or infusing one or more drugs to the patients's pelvic floor for treating various disorders.

U.S. Pat. No. 6,505,074 B2 (Boveja et al.) is directed to an implanted stimulus receiver coupled with an external stimulator for providing neuromodulation therapy. U.S. Pat. No. 6,449,512 B1 (Boveja) is directed to an implantable pulse generator for providing electrical stimulation therapy for urological disorders. The implanted pulse generator, though convenient, has the disadvantage that the internal battery will not last for a desired period of time, which can lead to repeated surgeries for generator replacement. The inductively coupled implanted stimulus receiver overcomes the disadvantage of implanted battery replacement, but patient convenience is an issue since a primary coil has to be kept in close proximity to an implanted secondary coil.

It would be desirable to have the advantages of both an IPG system and an inductively coupled system. The system and method disclosed, provides an improved method and system for adjunct therapy by providing a system that has the benefits of both systems, and has additional synergistic benefits not possible in the prior art. In the system of this invention, the patient can choose when to use an external inductively coupled system to conserve the battery life of the implanted module and receive higher levels of therapy.

### Urinary Incontinence

In considering the background of urinary urge incontinence, FIG. 1 shows a sagittal section of the human female pelvis showing the bladder 89 and urethra 13 in relation to other anatomic structures. Although FIG. 1 displays a female pelvis, the pulsed electrical stimulation therapy of the current invention applies equally to males. Urinary continence requires a relaxed bladder during the collecting phase and permanent closure of the urethra, whereas at micturition (urination), an intravesical pressure above the opening pressure of the simultaneously relaxing urethra has to be generated. These functions of the bladder 89 and urethra 13 are centrally coordinated and non-separable. At bladder filling, the sensation of urge is mediated by slowly adapting mechanoreceptors in the bladder wall and the same receptors provide the triggering signal for micturition and the main driving force for a sustained micturition contraction. The mechanoreceptors are, technically speaking, tension receptors. It has been found that they respond equally well to tension increases induced passively by bladder filling and those induced actively by a detrusor contraction. These receptors have high dynamic sensitivity and are easily activated by external pressure transients, as may occur during coughing or tapping of the abdominal wall. Their faithful response to active changes in bladder pressure is well illustrated.

When sufficiently activated, the mechanorecptors trigger a coordinated micturition reflex via a center in the upper pons 388, as depicted schematically in FIG. 2. The reflex detrusor 92 (muscle in the wall of the urinary bladder) contraction generates an increased bladder pressure and an even stronger activation of the mechanoreceptors. Their activity in turn reinforces the pelvic motor output to the bladder 89, which leads to a further increase in pressure and more receptor activation and so on. In this way, the detrusor contraction is to a large extent self generating once initiated. Such a control mechanism usually is referred to as a positive feedback, and it may explain the typical all-or-nothing behavior of the parasympathetic motor output to the bladder 89. Once urine enters the urethra 13, the contraction is further enhanced by reflex excitation from urethral receptors. Quantitatively, the bladder receptors are most important.

A great advantage of the positive feedback system is that it ascertains a complete emptying of the bladder during micturition. As long as there is any fluid left in the lumen, the intravesical pressure will be maintained above the threshold for the mechanoreceptors and thus provide a continuous driving force for the detrusor. A drawback with this system is that it can easily become unstable. Any stimulus that elicits a small burst of impulses in mechanoreceptor afferents may trigger a full-blown micturition reflex. To prevent this from happening during the filling phase, the neuronal system controlling the bladder is equipped with several safety devices both at the spinal and supraspinal levels.

The best-known spinal mechanism is the reflex control of the striated urethral sphincter 90, which increases its activity in response to bladder mechanoreceptor activation during filling. An analogous mechanism is Edvardsen's reflex, which involves machanoreceptor activation of inhibitory sympathetic neurons to the bladder 89. The sympathetic efferents have a dual inhibitory effect, acting both at the postganglionic neurons in the vesical ganglia and directly on the detrusor muscle of the bladder 89. The sphincter and sympathetic reflexes are automatically turned off at the spinal cord level during a normal micturition. At the supraspinal level, there are inhibitory connections from the cerebral cortex and hypothalamus to the pontine micturition center 88. The pathways are involved in the voluntary control of continence. Other inhibitory systems seem to originate from the pontine and medullary parts of the brainstem with at least partly descending connections.

Bladder over-activity and urinary urge incontinence may result from an imbalance between the excitatory positive feedback system of the bladder 89 and inhibitory control systems causing a hyperexcitable voiding reflex. Such an imbalance may occur after macroscopic lesions at many sites in the nervous system or after minor functional disturbances of the excitatory or inhibitory circuits. Urge incontinence due to detrusor instability seldom disappears spontaneously. The symptomatic pattern also usually is consistent over long periods.

Based on clinical experience, subtypes of urinary incontinence include, Phasic detrusor instability and uninhibited overactive bladder. Phasic detrusor instability is characterized by normal or increased bladder sensation, phasic bladder contractions occurring spontaneously during bladder filling or on provocation, such as by rapid filling, coughing, or jumping. This condition results from a minor imbalance between the bladder's positive-feedback system and the spinal inhibitory mechanisms. Uninhibited overactive bladder is characterized by loss of voluntary control of micturition and impairment of bladder sensation. The first sensation of filling is experienced at a normal or lowered volume and is almost immediately followed by involuntary micturition. The patient does not experience a desire to void until she/he is already voiding with a sustained detrusor contraction and a concomitant relaxation of the urethra, i.e., a well-coordinated micturition reflex. At this stage, she/he is unable to interrupt micturition voluntarily. The sensory disturbance of these subjects is not in the periphery, at the level of bladder mechanoreceptors, as the micturition reflex occurs at normal or even small bladder volumes. More likely, the suprapontine sensory projection to the cortex is affected. Such a site is consistent with the coordinated micturition and the lack of voluntary control. The uninhibited overactive bladder is present in neurogenic dysfunction.

Since bladder over-activity results from defective central inhibition, it seems logical to improve the situation by reinforcing some other inhibitory system. Patients with stress and urge incontinence are difficult to treat adequately. Successful therapy of the urge component does not influence the stress incontinence. While an operation for stress incontinence sometimes results in deterioration of urgency component. Electro stimulation using pulsed electrical stimulation is a logical alternative in mixed stress and urge incontinence, since the method improves urethral closure as well as bladder control. Drug treatment often is insufficient and, even when effective, does not lead to restoration of a normal micturition pattern.

Neuromodulation is a technique that applies pulsed electrical stimulation to the sacral nerves. A general diagram of spinal cord and sacral nerves 54 is shown in FIGS. 3A and 3B. One of the aim of this treatment modality is to achieve detrusor 392 inhibition by chronic electrical stimulation of afferent somatic sacral nerve fibers 54 via implanted electrodes in contact with sacral nerve fibers and connected to pulse generator means.

The rationale of this treatment modality is based on the existence of spinal inhibitory systems that are capable of interrupting a detrusor 392 contraction. Inhibition can be achieved by electrical stimulation of afferent anorectal branches of the pelvic nerve, afferent sensory fibers in the pudendal nerve and muscle afferents from the limbs. Most of these branches and fibers reach the spinal cord via the dorsal roots of the sacral nerves 54. Of the sacral nerve roots the S₃ root is the most practical for use in chronic pulsed electrical stimulation, although S₄ and S₂ along with S₃ may be stimulated.

### Other Urological Disorders

In addition to urinary incontinence, pulsed electrical stimulation of sacral nerve(s) and/or pudendal nerve(s) also provides therapy or alleviates symptoms for a broad group of urological or genito-urinary disorders such as prostatitis, prostatalgia and prostatodynia. Therapy may be provided using bilateral stimulation (FIG. 10A) or unilateral stimulation (FIG. 10B).

### Interstitial Cystitis

Interstitial cystitis is a painful and frequently debilitating condition of the urinary bladder. There are an estimated 700,000 cases of interstitial cystitis in the United States. Its symptoms include pelvic pain, dyspareunia, urinary urgency and frequency, nocturia, and small voided volumes with small bladder capacity. A prospective study that evaluated sacral neuromodulation for the treatment of refractory interstitial cystitis found that 94% of subjects implanted demonstrated a sustained improvement in symptoms. It was also shown that sacral neuromodulation decreased narcotic requirements in refractory interstitial cystitis. Also, in this study patients were overwhelmingly satisfied with the results of their trial of neuromodulation compared with their prior therapies.

### Fecal Incontinence

Fecal incontinence is a common disorder with a prevalence that rises with age. Individuals suffering from fecal incontinence find it distressing and socially incapacitating. The prevalence is estimated to be 3.5% for women and 2.3% for men. It has been shown that between four and six percent of women having a vaginal delivery will suffer from fecal incontinence.

Dietary manipulation, pharmacological drugs, pelvic floor physiotherapy as well as surgery are often used as combination treatment for patients suffering from fecal incontinence. A stoma (colostomy or ileostomy) is reserved for patients with end-stage fecal incontinence where available treatments have failed or are inappropriate due to comorbidities. While a stoma is successful in controlling fecal incontinence, it is associated with significant psychosocial and economic issues and stoma-related complications. Sacral nerve stimulation (SNS) is an innovative treatment for end-stage fecal incontinence and could obviate the need for a stoma.

The neural supply to the anorectal region is both somatic and autonomic. The superficial perineal nerve (branch of pudendal nerve) provides sensory fibers to the perineum, external genitalia as well as anal canal mucosa. Motor nerve supply to the pelvic floor and external anal sphincter is from the sacral plexus (S2-S4 level). The levator ani and puborectalis muscles are supplied on both the pelvic and perineal surfaces by direct branches from the nerve roots. The external anal sphincter receives its motor supply from the inferior rectal nerve (a branch of the pudendal nerve) and the deep perineal nerve (also a branch of the pudendal nerve) supplies the transverse perineal muscle and urethral sphincter.

The autonomic nerve supply is from both the sympathetic and parasympathetic systems. The sympathetic system is mainly inhibitory to colonic motility and excitatory to the internal anal sphincter. The supply is from the L1-L2 level via the hypogastric nerves. The parasympathetic supply is distributed via the sacral nerves (S2-S4) via the pelvic plexus and is excitatory to colonic motility as well as inhibitory to the internal anal sphincter. There is also an intrinsic nervous system of the colon and rectum with cell bodies within the colonic wall, but these can be affected by the autonomic system and local factors.

There appears to be a dual peripheral nerve supply (branches of the pudendal nerve and direct branches of sacral nerves) to the continence mechanism and the sacral spinal nerve is the most distal common location of this dual supply. Therefore, stimulation at this level can potentially excite both nerves. The basis for sacral nerve stimulation (SNS) is that by stimulating these sacral nerves, additional residual function of an inadequate pelvic floor musculature and pelvic organs can be recruited.

During SNS treatment for patients with urinary incontinence, some patients noticed improvement in any concurrent fecal incontinence also. The medical investigators found increased anorectal junction angulation, as well, as increased anal canal closure pressure as potential mechanisms to account for improvement of fecal continence.

### Mechanism of Action

One clinical study reported on the use of SNS in treating three patients with fecal incontinence. After 6 months, two patients regained complete continence while the third improved significantly. He noticed that the maximal anal squeeze pressure increased after stimulation. This improved continence was attributed to a direct nervous stimulation of the external anal sphincter. It was hypothesized that SNS stimulated the conversion of fast twitch, fatiguable type II muscle fibers in the external sphincter and pelvic floor to slow twitch type I fibers, based on the findings in previous studies.

Subsequent studies showed that mean resting anal pressure is raised after successful SNS treatment, suggesting that SNS might have an effect on the autonomic nervous system as well. In one study the rectal blood flow by laser Doppler flowmetry showed increased rectal blood flow after stimulation, and attributed this to modulation of the autonomic system that affects the blood vessels. More recent studies have shown reduced rectal sensory threshold and improved balloon expulsion time. These findings suggest SNS modulates the normal anorectal reflexes, as well as, stimulated the sacral nerve motor outflow.

In summery, sacral nerve stimulation achieves its effect through several physiological mechanisms. It stimulates the motor output from the sacral nerves and pudendal nerve, modulates the local spinal reflex arcs, and modulates the autonomic supply to the rectum and pelvic floor as well as spinal tracts to the higher center in the brain.

### Patient Screening

In one aspect of the invention, peripheral nerve evaluation (PNE) may be used to determine the feasibility of implanting an electrode into the sacral foramina (acute stage) and to assess the benefits after a period of stimulation (subchronic stage) of the sacral nerves. Screening of patients with fecal incontinence through PNE allows preselection of patients who are likely to have a good response to SNS. Acute PNE serves to locate the optimal sacral spinal nerve that will elicit contractions of the striated pelvic floor muscles, thus establishing the integrity of the sacral spinal nerves.

The procedure can be done under local or general anesthesia. During the procedure, with the patient lying prone, sheathed needles are inserted into the dorsal foramina of S2, S3 and S4 bilaterally under sterile conditions, such that the electrodes are placed close to where the sacral spinal nerves enter the pelvic cavity through the ventral sacral foramina and proximal to the sacral plexus. Intermittent stimulation with graduated amplitudes is applied to a needle until a muscle contraction is obtained. If acute peripheral nerve evaluation (PNE) successfully elicits the required reaction, an electrode is inserted for the subchronic stage of PNE.

For the subchronic stage, a tined lead is implanted with the electrodes in the appropriate position, which if successful, will be retained and connected to the permanent implant. The electrodes of the tined lead are connected to a temporary external pulse generator, via an external extension cable. Stimulation is applied, which can be turned off during micturition and defecation. The patient is evaluated typically for a minimum period of 7 days of subchronic PNE for improvement in fecal continence.

Patients who have significant improvement after subchronic PNE can be implanted with a permanent implantable pulse generator (IPG). The spinal nerve site which is chosen is the one that has previously been demonstrated to be therapeutically effective during the test stimulation phase. Using a tined lead during PNE has the advantage that it does not require any change of electrode, which is already in the optimal location in the sacral canal. The electrode is then connected subcutaneously via an extension wire to the implantable pulse generator, which is then placed in a subcutaneous location in the lower abdomen or gluteal area.

### Neuromodulation

As shown in conjunction with FIGS. 4 and 5, most nerves in the human body are composed of thousands of fibers, of different sizes designated by groups A, B and C. A peripheral nerve, for example, may have approximately 100,000 fibers of the three different types, each carrying signals. Each axon or fiber of that nerve conducts only in one direction, in normal circumstances. The A and B fibers are myelinated (i.e., have a myelin sheath, constituting a substance largely composed of fat, whereas the C fibers are unmyelinated.

A commonly used nomenclature for peripheral nerve fibers, using Roman and Greek letters, is given in the table below:

| | External | Conduction |
|---|---|---|
| Group | Diameter (μm) | Velocity (m/sec) |
| Myelinated Fibers | | |
| Aα or IA | 13-20 | 80-120 |
| Aβ: IB | 10-15 | 60-75 |
| II | 5-15 | 30-75 |
| Aγ | 3-8 | 15-40 |
| Aδ or III | 3-8 | 10-30 |
| B | 1-3 | 5-15 |
| Unmyelinted fibers | | |
| C or IV | 0.2-1.5 | 0.5-2.5 |

The diameters of group A and group B fibers include the thickness of the myelin sheaths. Group A is further subdivided into alpha, beta, gamma, and delta fibers in decreasing order of size. There is some overlapping of the diameters of the A, B, and C groups because physiological properties, especially the form of the action potential, are taken into consideration when defining the groups. The smallest fibers (group C) are unmyelinated and have the slowest conduction rate, whereas the myelinated fibers of group B and group A exhibit rates of conduction that progressively increase with diameter. Group B fibers are not present in the nerves of the limbs; they occur in white rami and some cranial nerves.

Compared to unmyelinated fibers, myelinated fibers are typically larger, conduct faster, have very low stimulation thresholds, and exhibit a particular strength-duration curve or respond to a specific pulse width versus amplitude for stimulation. The A and B fibers can be stimulated with relatively narrow pulse widths, from 50 to 200 microseconds (μs), for example. The A fiber conducts slightly faster than the B fiber and has a slightly lower threshold. The C fibers are very small, conduct electrical signals very slowly, and have high stimulation thresholds typically requiring a wider pulse width (300-1,000 μs) and a higher amplitude for activation. Selective stimulation of only A and B fibers is readily accomplished. The requirement of a larger and wider pulse to stimulate the C fibers, however, makes selective stimulation of only C fibers, to the exclusion of the A and B fibers, virtually unachievable inasmuch as the large signal will tend to activate the A and B fibers to some extent as well.

Stimulation of individual fibers is shown in conjunction with FIGS. 6A, 6B and 7. A nerve cell can be excited by increasing the electrical charge within the neuron, thus increasing the membrane potential inside the nerve with respect to the surrounding extracellular fluid. The threshold stimulus intensity is defined as that value at which the net inward current (which is largely determined by Sodium ions) is just greater than the net outward current (which is largely carried by Potassium ions), and is typically around −55 mV inside the nerve cell relative to the outside (critical firing threshold). If however, the threshold is not reached, the graded depolarization will not generate an action potential and the signal will not be propagated along the axon. This fundamental feature of the nervous system i.e., its ability to generate and conduct electrical impulses, can take the form of action potentials, which are defined as a single electrical impulse passing down an axon. This action potential (nerve impulse or spike) is an “all or nothing” phenomenon, that is to say once the threshold stimulus intensity is reached, an action potential will be generated.

FIG. 6A illustrates a segment of the surface of the membrane of an excitable cell. Metabolic activity maintains ionic gradients across the membrane, resulting in a high concentration of potassium (K⁺) ions inside the cell and a high concentration of sodium (Na⁺) ions in the extracellular environment. The net result of the ionic gradient is a transmembrane potential that is largely dependent on the K⁺ gradient. Typically in nerve cells, the resting membrane potential (RMP) is slightly less than 90 mV, with the outside being positive with respect to inside.

To stimulate an excitable cell, it is only necessary to reduce the transmembrane potential by a critical amount. When the membrane potential is reduced by an amount ΔV, reaching the critical or threshold potential (TP); which is shown in conjunction with FIG. 6B. When the threshold potential (TP) is reached, a regenerative process takes place: sodium ions enter the cell, potassium ions exit the cell, and the transmembrane potential falls to zero (depolarizes), reverses slightly, and then recovers or repolarizes to the resting membrane potential (RMP).

For a stimulus to be effective in producing an excitation, it must have an abrupt onset, be intense enough, and last long enough. These facts can be drawn together by considering the delivery of a suddenly rising cathodal constant-current stimulus of duration d to the cell membrane as shown in FIG. 6B. Cell membranes can be reasonably well represented by a capacitance C, shunted by a resistance R as shown by a simplified electrical model in FIG. 7.

When the stimulation pulse is strong enough, an action potential will be generated and propagated. Immediately after the spike of the action potential there is a refractory period when the neuron is either unexcitable (absolute refractory period) or only activated to sub-maximal responses by supra-threshold stimuli (relative refractory period). The absolute refractory period occurs at the time of maximal Sodium channel inactivation while the relative refractory period occurs at a later time when most of the Na⁺ channels have returned to their resting state by the voltage activated K⁺ current. The refractory period has two important implications for action potential generation and conduction. First, action potentials can be conducted only in one direction, away from the site of its generation, and secondly, they can be generated only up to certain limiting frequencies.

Pulsed electrical stimulation induces nerve impulses in the form of action potentials in the nerve fibers. These electrical signals travel along the nerve fibers. The information in the nervous system is coded by frequency of firing rather than the size of the individual action potentials. The bottom portion of FIG. 8 shows a train of action potentials 7. Shown in conjunction with FIG. 9, the rate of action potential generation depends on the magnitude of the depolarizing current. Thus, the firing frequency of action potentials reflects the magnitude of the depolarizing current. This is one way that stimulation intensity is encoded in the nervous system, as shown in FIG. 9. Although firing frequency increases with the amount of depolarizing current, there is a limit to the rate at which neurons can generate action potentials, depending on the absolute refractory period and the relative refractory period.

In neuromodulation of the current invention, the entire innervation system should be intact. As shown schematically in FIG. 10A, the procedure consists of placing the distal portion of the lead 40 with electrodes 61,62 in one of the sacral foraman as close to the pelvic plexus and pudendal nerve as possible, and connecting the lead 40 to the implanted stimulator 75, which is placed subcutaneously. Bilateral lead placement for bilateral stimulation is depicted in FIG. 10B. The hypothesis behind neuromodulation of the sacral roots (sensory and motor) is to correct, by use of the regulating electrical impulses, the dys-synergic activities of the cholinergic, adrenergic, and motor reflex pathways that initiate vesical storage and micturition. Although some theories have been developed that explain the effects of neuromodulation, most of the results are based on empiric findings in human studies. Some animal experiments and electrophysiologic studies in humans show there is a spinal inhibitory action through the afferent branches of the pelvic and pudendal nerves. It is not clear whether neuromodulation primarily influences the micturiction center located near the thalamus in the brain. Some maintain that there is a direct correction of the dys-synergis of the pelvic floor (pudendal nerve) by influencing the abnormal contractility of the pelvic floor.

A neurophysiological explanation for the effectiveness of this treatment modality in detrusor instability is based on animal experiments and electrophysiological studies in humans. Electrical stimulation for the treatment of urinary incontinence has evolved over the past 40 years. The mechanism of action of electrical stimulation was investigated initially in animal models. Over 100 years ago, Griffiths demonstrated relaxation of a contracted detrusor during stimulation of the proximal pudendal nerve in the cat model and further work clarified the role of pudendal afferents in relation of the detrusor. Spinal inhibitory systems capable of interrupting a detrusor contraction can be activated by electrical stimulation of afferent anorectal branhes of the pelvic nerve, afferent sensory fibers in the pudendal nerve and muscle afferents from the limbs. The effectiveness of neuromodulation in humans has been objectively demonstrated by urodynamic improvement, especially in light of the fact that such effects have not been noted in drug trials.

Neuromodulation also acts on neural reflexes but does so internally by stimulation of the sacral nerves 54. Sacral nerves 54 stimulation is based on research dedicated to the understanding of the voiding reflex as well as the role and influence of the sacral nerves 54 on voiding behavior. This research led to the development of a technique to modulate dysfunctional voiding behavior through sacral nerve stimulation. It is thought that sacral nerve stimulation induces reflex mediated inhibitory effects on the detrusor through afferent and/or efferent stimulation of the sacral nerves 54.

Even though the precise mechanism of action of electrical stimulation in humans is not fully understood, it has been shown that sensory input traveling through the pudendal nerve can inhibit detrusor activity in humans. It is generally believed that non-implanted electrical stimulation works by stimulating the pudendal nerve afferents, with the efferent outflow causing contraction of the striated pelvic musculature. There is also inhibition of inappropriate detrusor activity, though the afferent mechanism has yet to be clarified. There is consensus that the striated musculature action is able to provide detrusor inhibiton in this setting.

In summary, the rationale for neuromodulation in the management of such patients is the observation that stimulation of the sacral nerves via electrical stimulation can inhibit inappropriate neural reflex behavior.

In the method and system of this invention, pulsed electrical stimulation is provided using both implanted and external components. The pulse generator may be implanted in the body, or may be external to the body. In one aspect the external components may be networked over a wide area network, for remote interrogation and remote programming of stimulation parameters.

### SUMMARY OF THE INVENTION

The present invention has certain objects. That is, various embodiments of the present invention provide solution to one or more problems exiting in the prior art, including the problems of: a) testing the effectiveness of the therapy with a device and then implanting a different system to provide therapy; b) patient requires periodic surgeries to replace system at the end of battery-life, (typical battery life is 3-6 years); c) patient is not able to easily change between an implanted, external, or integrated system; d) patient is unable to make use of ‘cumulative effect’ of therapy to reduce/eliminate disorders, in addition to just ‘event’ based therapy due to limited battery life of exiting systems; e) frequent patient visits to clinics/physician office to monitor the device; f) the titration of therapy is a long and drawn out.

The method and system of current invention provides pulsed electrical stimulation to provide therapy for urinary incontinence, fecal incontinence, and urological disorders. The urological disorders include overflow incontinence, stress incontinence, idiopathic chronic urinary retention, interstitial cystitis, neuro-urological disorder, vesico-urethral dysfunctions, bladder inflammation, bladder pain, pelvic pain, and genito-urinary disorders such as prostatitis, prostatalgia, and prostatodynia. The stimulation is to sacral nerve(s) or its branches or portions thereof, to provide therapy. The method and system comprises both implantable and external components. The power source may also be external or implanted in the body. The system to provide selective stimulation to sacral nerve(s) or branches may be selected from a group consisting of:

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a programmable implantable pulse generator (IPG);

e) a combination implantable device comprising both a stimulus-receiver and a programmable IPG; and

f) an IPG comprising a rechargeable battery.

In one aspect of the invention, the selective stimulation is to sacral nerve(s) or branches or parts thereof to provide therapy.

In another aspect of the invention, the electrical pulses to sacral nerve(s) or branches may be provided unilaterally, or bilaterally.

In another aspect of the invention, rectangular and/or complex pulses are used.

In another aspect of the invention, these predetermined/pre-packaged programs may be used to provide therapy.

In another aspect of the invention, predetermined/pre-packaged programs can be modified.

In another aspect of the invention, the stimulation may be unidirectional.

In another aspect of the invention, blocking may be provided to selected branches.

In another aspect of the invention, the pulse generator may be implanted in the body.

In another aspect of the invention, the implanted pulse generator is adapted to be re-chargable via an external power source.

In another aspect of the invention, the external components such as the external stimulator, or programmer comprise telemetry means adapted to be networked, for remote interrogation or remote programming of the device.

In another aspect of the invention, the implanted lead body may be made of a material selected from the group consisting of polyurethane, silicone, and silicone with polytetrafluoroethylene (PTFE).

In yet another aspect of the invention, the implanted lead comprises at least one electrode selected from the group consisting of platinum, platinum/iridium alloy, platinum/iridium alloy coated with titanium nitride, and carbon.

Various other features, objects and advantages of the invention will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram of the sagittal section of the female pelvis, showing the relationship between various anatomic structures.

FIG. 2 is a schematic diagram showing physiological control of micturition.

FIG. 3A is a diagram showing anatomic relationships of spinal nerves and sacral region.

FIG. 3B is a diagram showing the sacral nerves (S₁-S₄), and selected branches including the pudendal nerve.

FIG. 4 is a diagram of the structure of a nerve.

FIG. 5 is a table showing details of nerve fiber characteristics.

FIGS. 6A and 6B show an action potential across a nerve fiber.

FIG. 7 is a schematic illustration of electrical properties of the nerve fiber membrane.

FIG. 8 is an illustration showing a train of action potentials.

FIG. 9 is a diagram depicting action potentials in response to changing depolarization currents.

FIG. 10A is a diagram showing schematically the placement of the implanted lead in contact with the sacral nerve(s) or branches, depicting unilateral stimulation.

FIG. 10B is a diagram showing schematically the placement of the implanted leads in contact with the sacral nerve(s) or branches, depicting bilateral stimulation, with a dual channel stimulator.

FIG. 11 is a simplified block diagram depicting supplying amplitude and pulse width modulated electromagnetic pulses to an implanted coil.

FIG. 12 shows coupling of the external stimulator and the implanted stimulus-receiver.

FIG. 13 is a schematic of the passive circuitry in the implanted lead-receiver.

FIG. 14A is a schematic of an alternative embodiment of the implanted lead-receiver.

FIG. 14B is another alternative embodiment of the implanted lead-receiver.

FIG. 15 is a top-level block diagram of the external stimulator and proximity sensing mechanism.

FIG. 16 is a diagram showing the proximity sensor circuitry.

FIG. 17 shows the pulse train to be transmitted to the nerve tissue.

FIG. 18 shows the ramp-up and ramp-down characteristic of the pulse train.

FIGS. 19A and 19B are schematic diagrams of the implantable leads.

FIGS. 20A, 20B, and 20C depicts various types of electrodes at the distal end of a lead.

FIGS. 21A, 21B, 21C, 21D, and 21E are diagrams depicting various types of anchoring sleeves.

FIG. 22A is diagram depicting stimulating electrode-tissue interface.

FIG. 22B is diagram depicting an electrical model of the electrode-tissue interface.

FIG. 23 is a schematic diagram showing the implantable lead and one form of stimulus-receiver.

FIG. 24 is a schematic block diagram showing a system for neuromodulation of nerve tissue, with an implanted component which is both RF coupled and contains a capacitor power source.

FIG. 25 is a simplified block diagram showing control of an implantable neurostimulator with a magnet.

FIG. 26 is a schematic diagram showing implementation of a multi-state converter.

FIG. 27 is a schematic diagram depicting digital circuitry for state machine.

FIG. 28A is a simplified block diagram of the implantable pulse generator.

FIG. 28B is a simplified block diagram of the implantable pulse generator, depicting a dual channel stimulator.

FIG. 28C is a simplified block diagram of the implantable pulse generator, depicting a dual channel stimulator with sensing.

FIG. 29 is a functional block diagram of a microprocessor-based implantable pulse generator.

FIG. 30 shows details of implanted pulse generator.

FIGS. 31A and 31B show details of digital components of the implantable circuitry.

FIG. 32A shows a schematic diagram of the register file, timers and ROM/RAM.

FIG. 32B shows datapath and control of custom-designed microprocessor based pulse generator.

FIG. 33 is a block diagram for generation of a pre-determined stimulation pulse.

FIG. 34 is a simplified schematic for delivering stimulation pulses.

FIG. 35 is a circuit diagram of a voltage doubler.

FIG. 36A is a diagram depicting ramping-up of a pulse train.

FIG. 36B depicts rectangular pulses.

FIGS. 36C, 36D, and 36E depict multi-step pulses.

FIGS. 36F, 36G, and 36H depict complex pulse trains.

FIG. 36-I depicts the use of tripolar electrodes.

FIGS. 36J and 36K depict step pulses used in conjunction with tripolar electrodes.

FIGS. 36L and 36M depict biphasic pulses used in conjunction with tripolar pulses.

FIGS. 36N and 36-O depict modified square pulses to be used in conjunction with tripolar electrodes.

FIG. 37 depicts an implantable system with tripolar lead for selective unidirectional blocking of nerve stimulation;

FIG. 38 depicts selective unidirectional blocking with nerve stimulation.

FIGS. 39A and 39B are diagrams showing communication of programmer with the implanted stimulator.

FIGS. 40A and 40B show diagrammatically encoding and decoding of programming pulses.

FIG. 41 is a simplified overall block diagram of implanted pulse generator (IPG) programmer.

FIG. 42 shows a programmer head positioning circuit.

FIG. 43 depicts typical encoding and modulation of programming messages.

FIG. 44 shows decoding one bit of the signal from FIG. 43.

FIG. 45 shows a diagram of receiving and decoding circuitry for programming data.

FIG. 46 shows a diagram of receiving and decoding circuitry for telemetry data.

FIG. 47 is a block diagram of a battery status test circuit.

FIG. 48 is a diagram showing the two modules of the implanted pulse generator (IPG) of one embodiment.

FIG. 49A depicts coil around the titanium case with two feedthroughs for a bipolar configuration.

FIG. 49B depicts coil around the titanium case with one feedthrough for a unipolar configuration.

FIG. 49C depicts two feedthroughs for the external coil which are common with the feedthroughs for the lead terminal.

FIG. 49D depicts one feedthrough for the external coil which is common to the feedthrough for the lead terminal.

FIG. 50 shows a block diagram of an implantable stimulator which can be used as a stimulus-receiver or an implanted pulse generator with rechargeable battery.

FIG. 51 is a block diagram highlighting battery charging circuit of the implantable stimulator of FIG. 50.

FIG. 52 is a schematic diagram highlighting stimulus-receiver portion of implanted stimulator of one embodiment.

FIG. 53A depicts bipolar version of stimulus-receiver module.

FIG. 53B depicts unipolar version of stimulus-receiver module.

FIG. 54 depicts power source select circuit.

FIG. 55A shows energy density of different types of batteries.

FIG. 55B shows discharge curves for different types of batteries.

FIG. 56 depicts externalizing recharge and telemetry coil from the titanium case.

FIGS. 57A and 57B depict recharge coil on the titanium case with a magnetic shield in-between.

FIG. 58 shows in block diagram form an implantable rechargable pulse generator.

FIG. 59 depicts in block diagram form the implanted and external components of an implanted rechargable system.

FIG. 60 depicts the alignment function of rechargable implantable pulse generator.

FIG. 61 is a block diagram of the external recharger.

FIG. 62 depicts remote monitoring of stimulation devices.

FIG. 63 is an overall schematic diagram of the external stimulator, showing wireless communication.

FIG. 64 is a schematic diagram showing application of Wireless Application Protocol (WAP).

FIG. 65 is a simplified block diagram of the networking interface board.

FIGS. 66A and 66B is a simplified diagram showing communication of modified PDA/phone with an external stimulator via a cellular tower/base station.

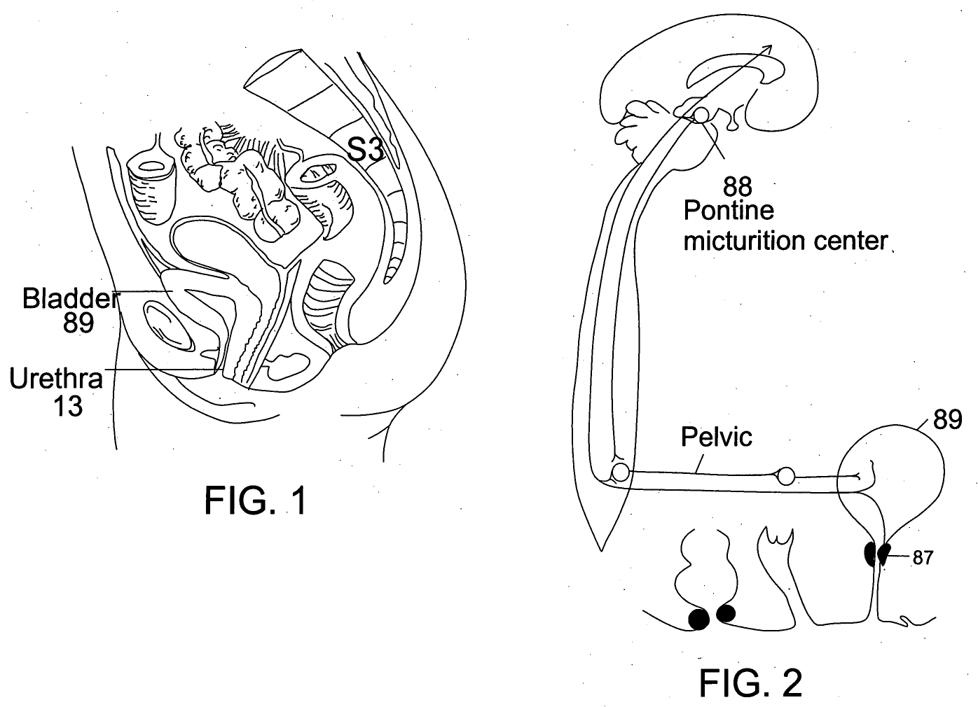

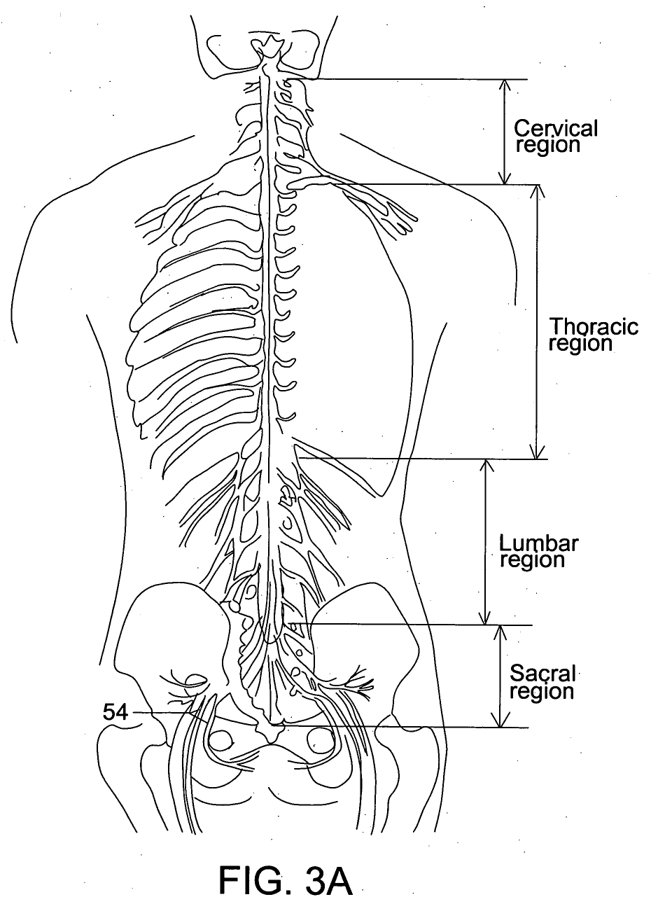

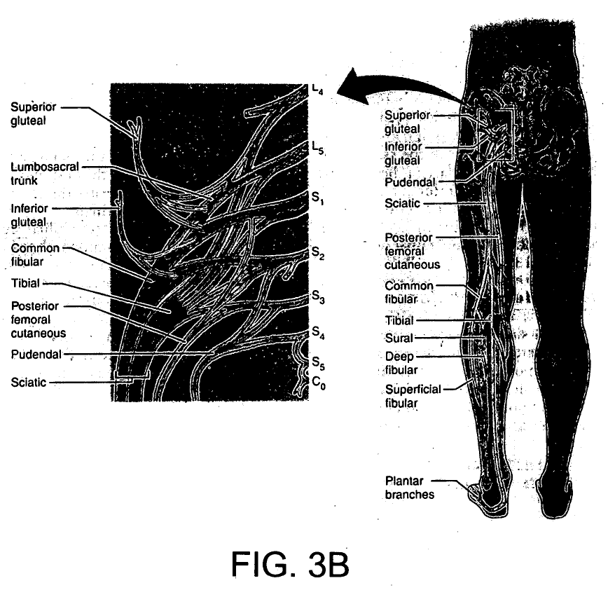

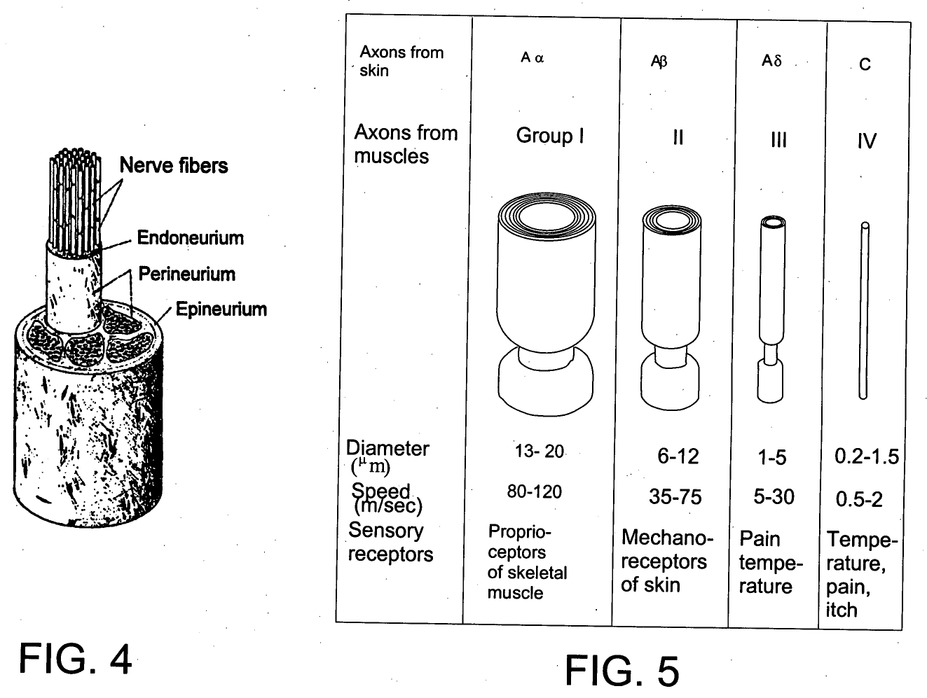

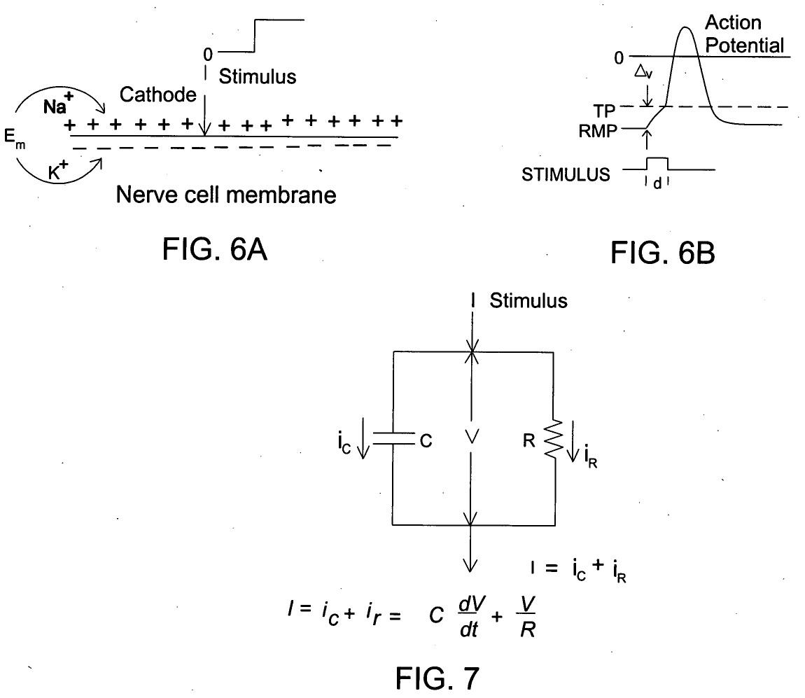

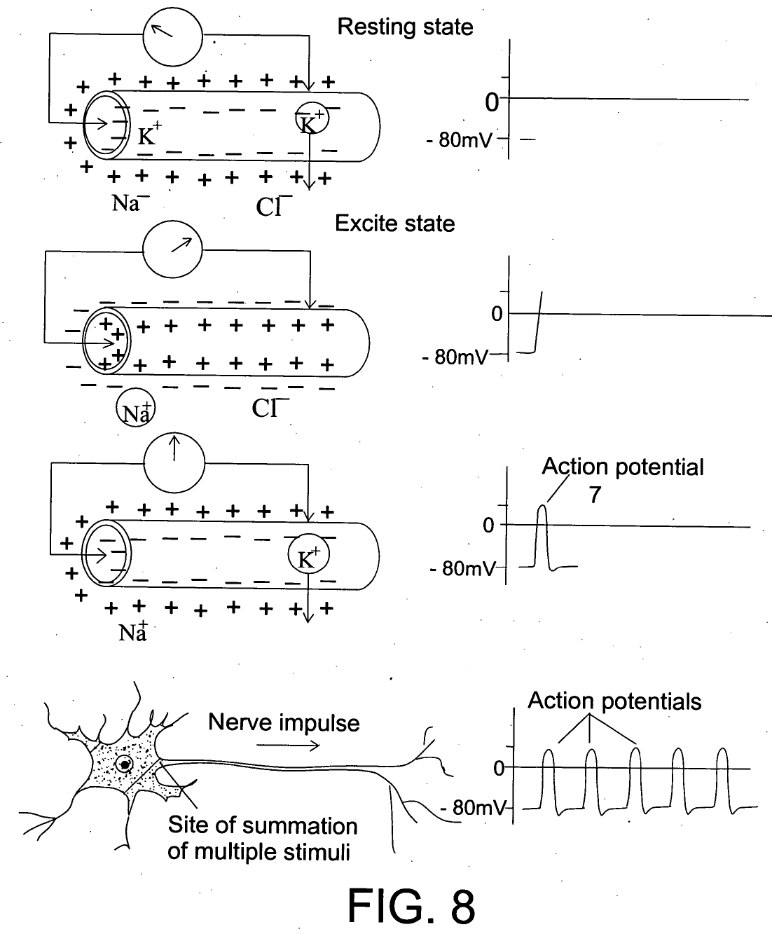

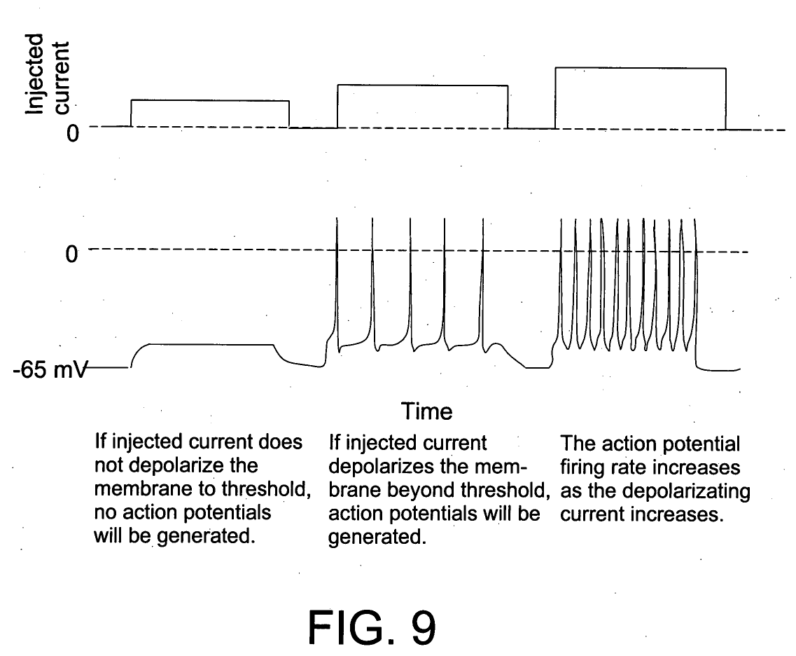

### DETAILED DESCRIPTION OF THE INVENTION

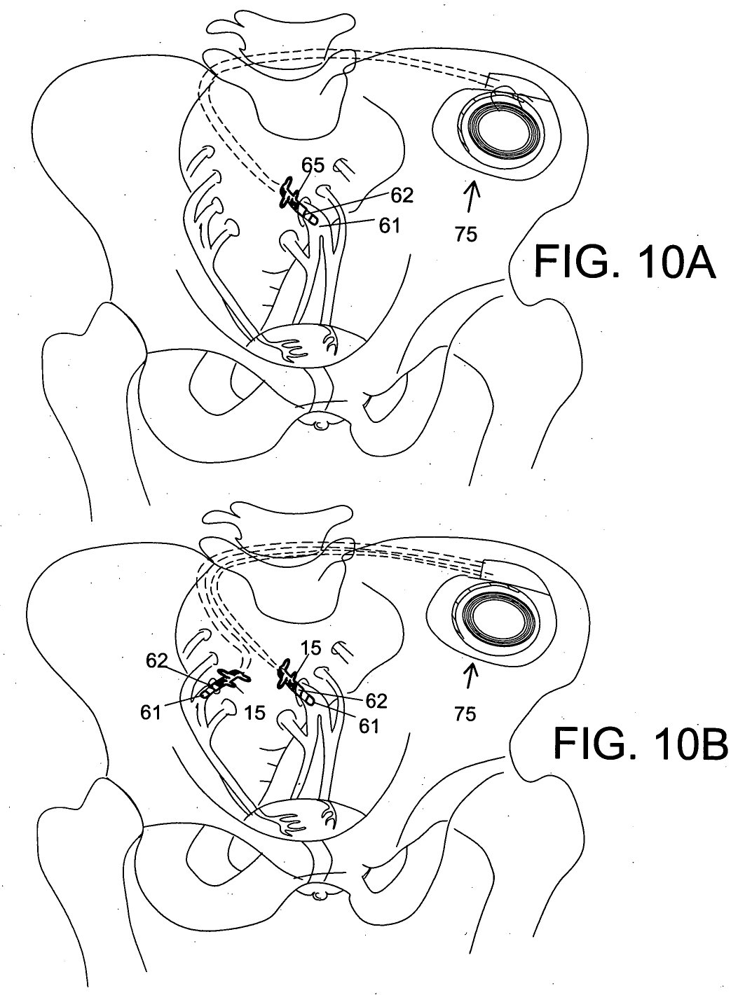

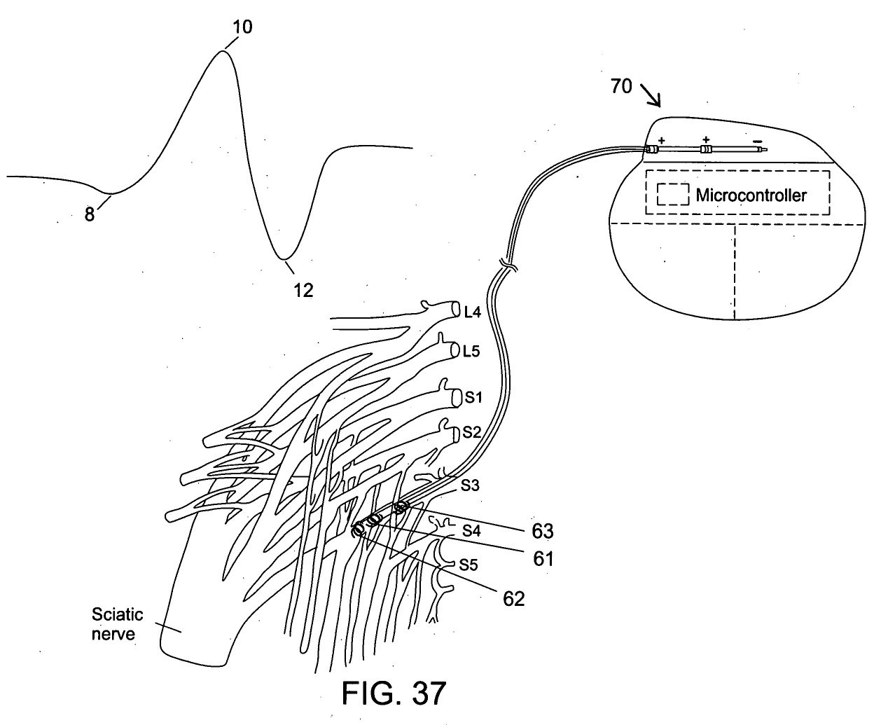

The method and system of the current invention delivers pulsed electrical stimulation, to provide therapy for urinary incontinence, urological disorders and/or fecal incontinence. The urological disorders include overflow incontinence, stress incontinence, idiopathic chronic urinary retention, interstitial cystitis, neuro-urological disorder, vesico-urethral dysfunctions, bladder inflammation, bladder pain, pelvic pain, and genito-urinary disorders such as prostatitis, prostatalgia, and prostatodynia. The electrical stimulation is delivered usually to S₃ (shown in FIGS. 10A and 37), but may be to S₄ or other sacral nerves or branches such as the pudendal nerves or perineal nerves. The electrode placement and stimulation may be unilateral (FIG. 10A) or bilateral (FIG. 10B). The method and system comprises both implantable and external components.

For implantation of the system, an incision is made and the distal portion of the lead is implanted in the tissue with electrodes in contact with the nerve tissue to be stimulated. The terminal portion of the lead is tunneled subcutaneously to a site where the pulse generator means is implanted, which is usually in the lower abdominal area (or may be in the gluteal region). The pulse generator (or stimulus-receiver) means is connected to the proximal end of the lead, placed in a subcutaneous pocket, and the tissues are surgically closed in layers (FIG. 10A). Stimulation therapy can be applied after the tissues are healed from the surgery. Stimulation can be applied in bipolar mode or in unipolar mode where the pulse generator can is used as the anode.

In the method and system of this invention, the pulse generator means may be implanted in the body or may be external to the body. Also, the power source may be external, implantable, or a combination device.

In the method of this invention, a simple and cheap pulse generator may be used to test a patient's response to neuromodulation therapy. As one example only, an implanted stimulus-receiver in conjunction with an external stimulator may be used initially to test patient's response. If the patient responds well, then at a later time, the pulse generator may be exchanged for a more elaborate implanted pulse generator (IPG) model, keeping the same lead. Some examples of stimulation and power sources that may be used interchangeably with the same lead for the practice of this invention, and disclosed in this Application, include:

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a programmable implantable pulse generator (IPG);

e) a combination implantable device comprising both a stimulus-receiver and a programmable IPG; and

f) an IPG comprising a rechargeable battery.

As another example, a cheap programmer-less IPG may be implanted initially to test the efficacy of neuromodulation therapy in the patient. If the patient responds well, the simple programmer-less IPG may be replaced with a higher functionality (and more expensive) version of IPG at a future time.

Also as disclosed later, the external components such as a programmer, or the external pulse generator, may comprise a telemetry module for remote communication over a wide area network such as the internet. This would provide means of remotely interrogating the device, or loading or activating new programs from a remote location.

Even though the pulse generator means are interchangeble, the lead(s) is implanted only once. The proximal (or terminal) portion of the lead is plugged into the pulse generator means. The distal portion of the lead comprises two, or three, or four electrodes for delivering electrical stimulation. As described earlier, the pulsed electrical stimulation may be to one of several nerves, however for purposes of describing the system, the stimulation site is referred to as simply “sacral nerves 54”. It is to be understood that the “sacral nerves 54” includes sacral nerves S₁, S₂, S₃, S₄, pudendal nerve, superior gluteal nerve, lumbo-sacral trunk, inferior gluteal nerve, common fibular nerve, tibial nerve, posterior femoral cutaneous nerve, sciatic nerve, and obturator nerve. Additionally, stimulation may be provided unilaterally or bilaterally via two leads.

### Implanted Stimulus-Receiver with an External Stimulator

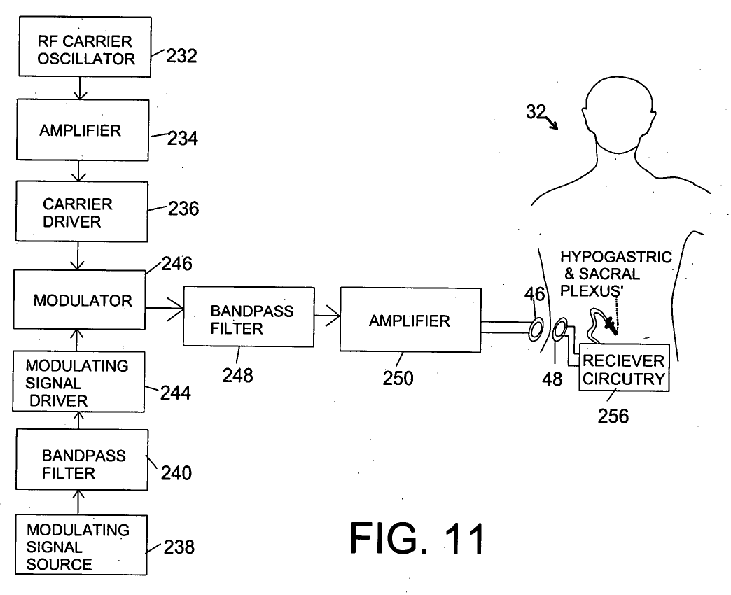

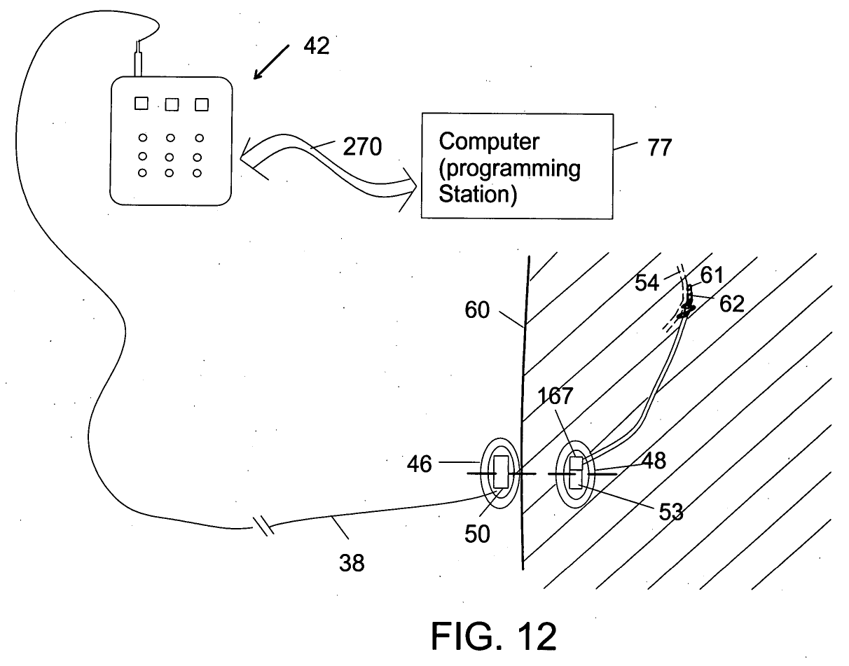

For an external power source, a passive implanted stimulus-receiver may be used. The appropriate stimulation of selected nerve fibers in the sacral and pelvic region, as performed by one embodiment of the method and system of this invention is shown schematically in FIG. 11, as a block diagram. A modulator 246 receives analog (sine wave) high frequency “carrier” signal and modulating signal. The modulating signal can be multilevel digital, binary, or even an analog signal. In this embodiment, mostly multilevel digital type i.e., pulse amplitude and pulse width modulated signals are used. The modulated signal is conditioned 254, amplified 250, and transmitted via a primary coil 46 which is external to the body. Shown in conjunction with FIG. 12, a secondary coil 48 of an implanted stimulus-receiver, receives, demodulates, and delivers these pulses to the sacral nerves 54 via electrodes 61 and 62 (or a proximal pair). The receiver circuitry 256 is described later.

The carrier frequency is optimized. One preferred embodiment utilizes electrical signals of around 1 Mega-Hertz, even though other frequencies can be used. Low frequencies are generally not suitable because of energy requirements for longer wavelengths, whereas higher frequencies are absorbed by the tissues and are converted to heat, which again results in power losses.

Also, shown in conjunction with FIG. 12, the primary (external) coil 46 of the external stimulator 42 is inductively coupled to the secondary (implanted) coil 48 of the implanted stimulus-receiver 34. The implantable stimulus-receiver has circuitry at the proximal end, and has stimulating electrodes at the distal end of the lead.

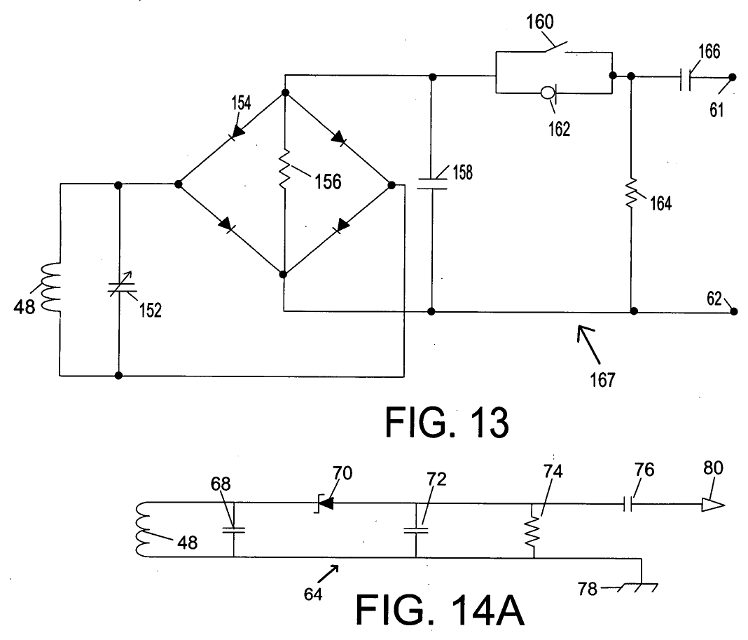

The circuitry contained in the proximal end of the implantable stimulus-receiver 34 is shown schematically in FIG. 13, for one embodiment. In this embodiment, the circuit uses all passive components. Approximately 25 turn copper wire of 30 gauge, or comparable thickness, is used for the primary coil 46 and secondary coil 48. This wire is concentrically wound with the windings all in one plane. The frequency of the pulse-waveform delivered to the implanted coil 48 can vary, and so a variable capacitor 152 provides ability to tune secondary implanted circuit 167 to the signal from the primary coil 46. The pulse signal from secondary (implanted) coil 48 is rectified by the diode bridge 154 and frequency reduction obtained by capacitor 158 and resistor 164. The last component in line is capacitor 166, used for isolating the output signal from the electrode wire. The return path of signal from cathode 61 will be through anode 62 placed in proximity to the cathode 61 for “Bipolar” stimulation. In this embodiment bipolar mode of stimulation is used, however, the return path can be connected to the remote ground connection (case) of implantable circuit 167, providing for much larger intermediate tissue for “Unipolar” stimulation. The “Bipolar” stimulation offers localized stimulation of tissue compared to “Unipolar” stimulation and is therefore, preferred in this embodiment. Unipolar stimulation is more likely to stimulate skeletal muscle in addition to nerve stimulation. The implanted circuit 167 in this embodiment is passive, so a battery does not have to be implanted.

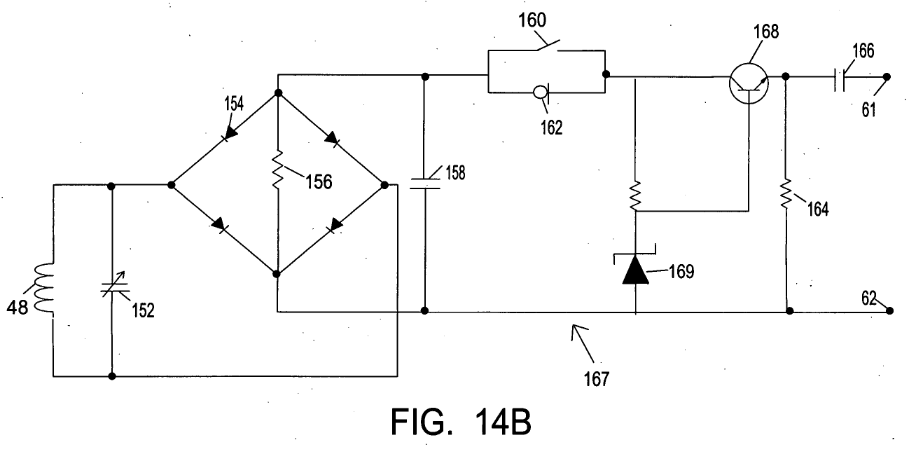

The circuitry shown in FIGS. 14A and 14B can be used as an alternative for the implanted stimulus-receiver. The circuitry of FIG. 14A is a slightly simpler version, and circuitry of FIG. 14B contains a conventional NPN transistor 168 connected in an emitter-follower configuration.

For therapy to commence, the primary (external) coil 46 is placed on the skin 60 on top of the surgically implanted (secondary) coil 48. An adhesive tape may be placed on the skin 60 and external coil 46 such that the external coil 46, is taped to the skin 60. For efficient energy transfer to occur, it is important that the primary (external) and secondary (internal) coils 46,48 be positioned along the same axis and be optimally positioned relative to each other. In this embodiment, the external coil 46 may be connected to proximity sensing circuitry 50. The correct positioning of the external coil 46 with respect to the internal coil 48 is indicated by turning “on” of a light emitting diode (LED) on the external stimulator 42.

Many different forms of proximity sensing mechanisms may be used. In one embodiment optimal placement of the external (primary) coil 46 may be done with the aid of proximity sensing circuitry incorporated in the system. Proximity sensing occurs utilizing a combination of external and implantable components. The implanted components contains a relatively small magnet composed of materials that exhibit Giant Magneto-Resistor (GMR) characteristics such as Samarium-cobalt, a coil, and passive circuitry. As was shown in conjunction with FIG. 12, the external coil 46 and proximity sensor circuitry 50 are rigidly connected in a convenient enclosure which is attached externally on the skin. The sensors measure the direction of the field applied from the magnet to sensors within a specific range of field strength magnitude. The dual sensors exhibit accurate sensing under relatively large separation between the sensor and the target magnet. As the external coil 46 placement is “fine tuned”, the condition where the external (primary) coil 46 comes in optimal position, i.e. is located adjacent and parallel to the subcutaneous (secondary) coil 48, along its axis, is recorded and indicated by a light emitting diode (LED) on the external stimulator 42.

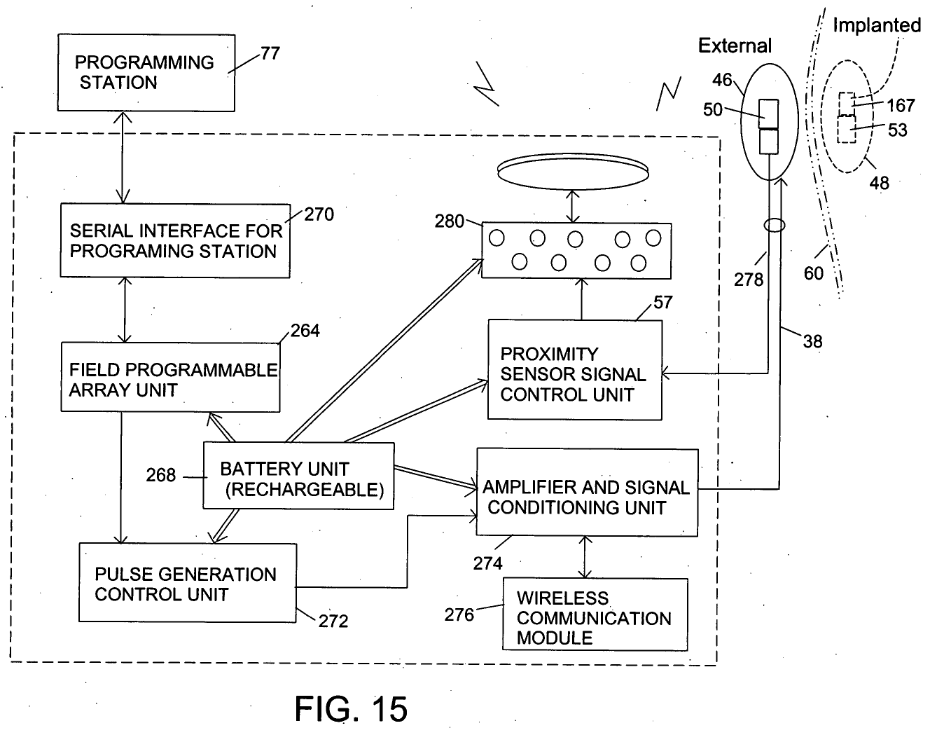

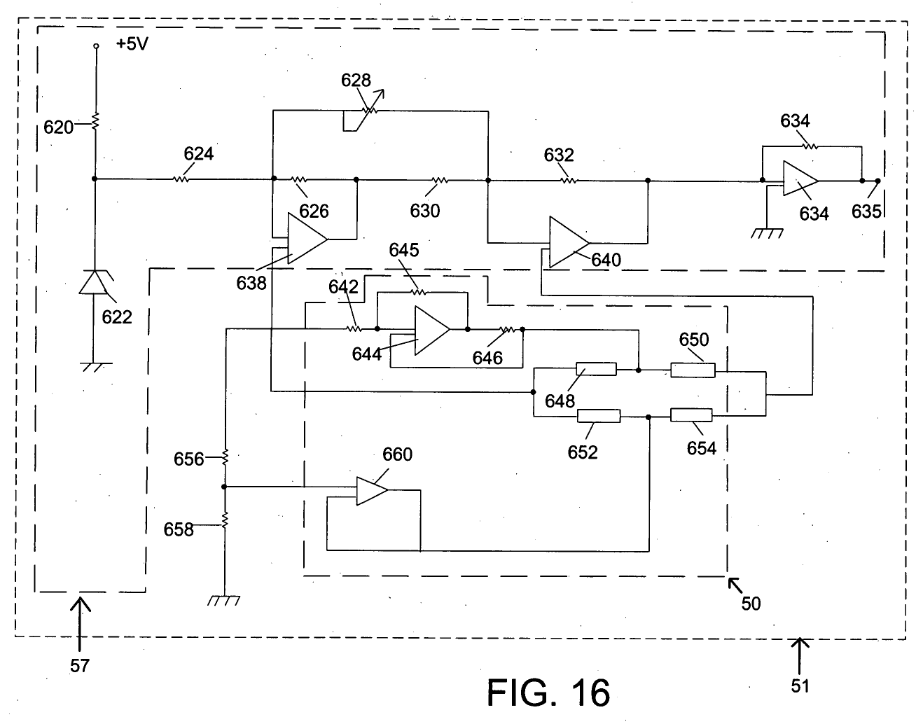

FIG. 15 shows an overall block diagram of the components of the external stimulator and the proximity sensing mechanism. The proximity sensing components are the primary (external) coil 46, supercutaneous (external) proximity sensors 648, 652 (FIG. 16) in the proximity sensor circuit unit 50, and a subcutaneous secondary coil 48 with a Giant Magneto Resister (GMR) magnet 53 associated with the proximity sensor unit. The proximity sensor circuit 50 provides a measure of the position of the secondary implanted coil 48. The signal output from proximity sensor circuit 50 is derived from the relative location of the primary and secondary coils 46, 48. The sub-assemblies consist of the coil and the associated electronic components, that are rigidly connected to the coil.

The proximity sensors (external) contained in the proximity sensor circuit 50 detect the presence of a GMR magnet 53, composed of Samarium Cobalt, that is rigidly attached to the implanted secondary coil 48. The proximity sensors, are mounted externally as a rigid assembly and sense the actual separation between the coils, also known as the proximity distance. In the event that the distance exceeds the system limit, the signal drops off and an alarm sounds to indicate failure of the production of adequate signal in the secondary implanted circuit 167, as applied in this embodiment of the device. This signal is provided to the location indicator LED 280.

FIG. 16 shows the circuit used to drive the proximity sensors 648, 652 of the proximity sensor circuit 50. The two proximity sensors 648, 652 obtain a proximity signal based on their position with respect to the implanted GMR magnet 53. This circuit also provides temperature compensation. The sensors 648, 652 are ‘Giant Magneto Resistor’ (GMR) type sensors packaged as proximity sensor unit 50. There are two components of the complete proximity sensor circuit. One component is mounted supercutaneously 50, and the other component, the proximity sensor signal control unit 57 is within the external stimulator 42. The resistance effect depends on the combination of the soft magnetic layer of magnet 53, where the change of direction of magnetization from external source can be large, and the hard magnetic layer, where the direction of magnetization remains unchanged. The resistance of this sensor 50 varies along a straight motion through the curvature of the magnetic field. A bridge differential voltage is suitably amplified and used as the proximity signal.

The Siemens GMR B6 (Siemens Corp., Special Components Inc., New Jersey) is used for this function in one embodiment. The maximum value of the peak-to-peak signal is observed as the external magnetic field becomes strong enough, at which point the resistance increases, resulting in the increase of the field-angle between the soft magnetic and hard magnetic material. The bridge voltage also increases. In this application, the two sensors 648, 652 are oriented orthogonal to each other.

The distance between the magnet 53 and sensor 50 is not relevant as long as the magnetic field is between 5 and 15 KA/m, and provides a range of distances between the sensors 648, 652 and the magnetic material 53. The GMR sensor registers the direction of the external magnetic field. A typical magnet to induce permanent magnetic field is approximately 15 by 8 by 5 mm³, for this application and these components. The sensors 648, 652 are sensitive to temperature, such that the corresponding resistance drops as temperature increases. This effect is quite minimal until about 100° C. A full bridge circuit is used for temperature compensation, as shown in temperature compensation circuit 50 of FIG. 16. The sensors 648, 652 and a pair of resistors 650, 654 are shown as part of the bridge network for temperature compensation. It is also possible to use a full bridge network of two additional sensors in place of the resistors 650, 654.

The signal from either proximity sensor 648, 652 is rectangular if the surface of the magnetic material is normal to the sensor and is radial to the axis of a circular GMR device. This indicates a shearing motion between the sensor and the magnetic device. When the sensor is parallel to the vertical axis of this device, there is a fall off of the relatively constant signal at about 25 mm. separation. The GMR sensor combination varies its resistance according to the direction of the external magnetic field, thereby providing an absolute angle sensor. The position of the GMR magnet can be registered at any angle from 0 to 360 degrees.

In the external stimulator 42 shown in FIG. 15, an indicator unit 280 which is provided to indicate proximity distance or coil proximity failure (for situations where the patch containing the external coil 46, has been removed, or is twisted abnormally etc.). Indication is also provided to assist in the placement of the patch. In case of general failure, a red light with audible signal is provided when the signal is not reaching the subcutaneous circuit. The indicator unit 280 also displays low battery status. The information on the low battery, normal and out of power conditions forewarns the user of the requirements of any corrective actions.

Also shown in FIG. 15, the programmable parameters are stored in a programmable logic 264. The predetermined programs stored in the external stimulator 42 are capable of being modified through the use of a separate programming station 77. The Programmable Array Logic Unit 264 and interface unit 270 are interfaced to the programming station 77. The programming station 77 can be used to load new programs, change the existing predetermined programs or the program parameters for various stimulation programs. The programming station is connected to the programmable array unit 75 (comprising programmable array logic 304 and interface unit 270) with an RS232-C serial connection (other connector types may be used). The main purpose of the serial line interface is to provide an RS232-C standard interface.

This method enables any portable computer with a serial interface to communicate and program the parameters for storing the various programs. The serial communication interface receives the serial data, buffers this data and converts it to a 16 bit parallel data. The programmable array logic 264 component of programmable array unit receives the parallel data bus and stores or modifies the data into a random access matrix. This array of data also contains special logic and instructions along with the actual data. These special instructions also provide an algorithm for storing, updating and retrieving the parameters from long-term memory. The programmable logic array unit 264, interfaces with long term memory to store the predetermined programs. All the previously modified programs can be stored here for access at any time, as well as, additional programs can be locked out for the patient. The programs consist of specific parameters and each unique program will be stored sequentially in long-term memory. A battery unit is present to provide power to all the components. The logic for the storage and decoding is stored in a random addressable storage matrix (RASM).

Conventional microprocessor and integrated circuits are used for the logic, control and timing circuits. Conventional bipolar transistors are used in radio-frequency oscillator, pulse amplitude ramp control and power amplifier. A standard voltage regulator is used in low-voltage detector. The hardware and software to deliver the pre-determined programs is well known to those skilled in the art.

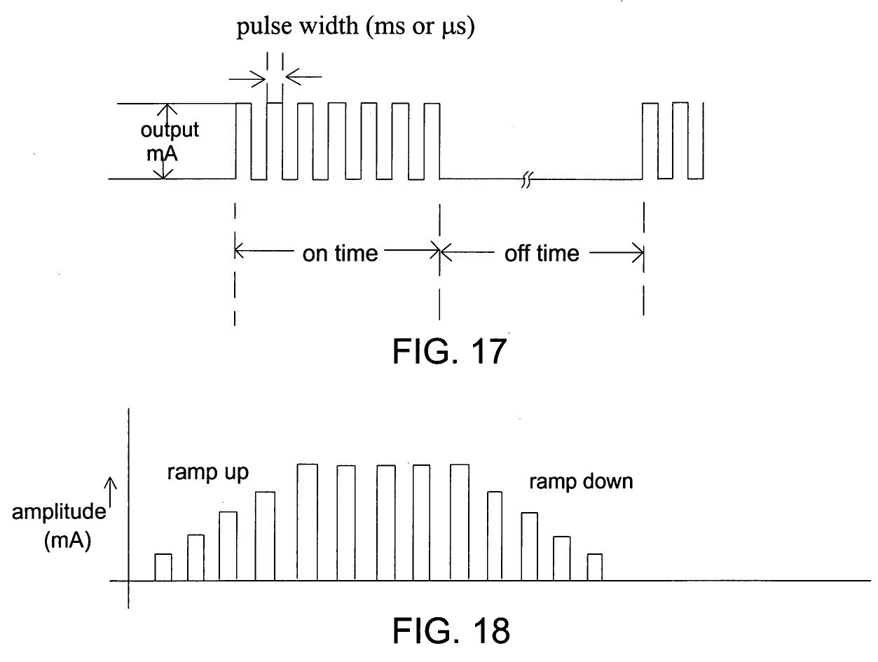

The pulses delivered to the nerve tissue for stimulation therapy are shown graphically in FIG. 17. In one embodiment as shown in FIG. 18, for patient comfort when the electrical stimulation is turned on, the electrical stimulation is ramped up and ramped down, instead of abrupt delivery of electrical pulses.

The selective stimulation to the sacral nerves can be performed in one of two ways. One method is to activate one of several “pre-determined” programs. A second method is to “custom” program the electrical parameters which can be selectively programmed, for specific therapy to the individual patient. The electrical parameters which can be individually programmed, include variables such as pulse amplitude, pulse width, frequency of stimulation, stimulation on-time, and stimulation off-time. Table two below defines the approximate range of parameters,

TABLE 2
Electrical parameter range delivered to the nerve

| PARAMER | RANGE |
|---|---|
| Pulse Amplitude | 0.1 Volt-15 Volts |
| Pulse width | 20 μS-5 mSec. |
| Frequency | 5 Hz-200 Hz |
| On-time | 10 Secs-24 hours |
| Off-time | 10 Secs-24 hours |

The parameters in Table 2 are the electrical signals delivered to the nerve tissue via the two electrodes 61,62 (distal and proximal) at the nerve tissue. It being understood that the signals generated by the external pulse generator 42 and transmitted via the primary coil 46 are larger, because the attenuation factor between the primary coil and secondary coil is approximately 10-20 times, depending upon the distance, and orientation between the two coils. Accordingly, the range of transmitted signals of the external pulse generator 42 are approximately 10-20 times larger than shown in Table 2.

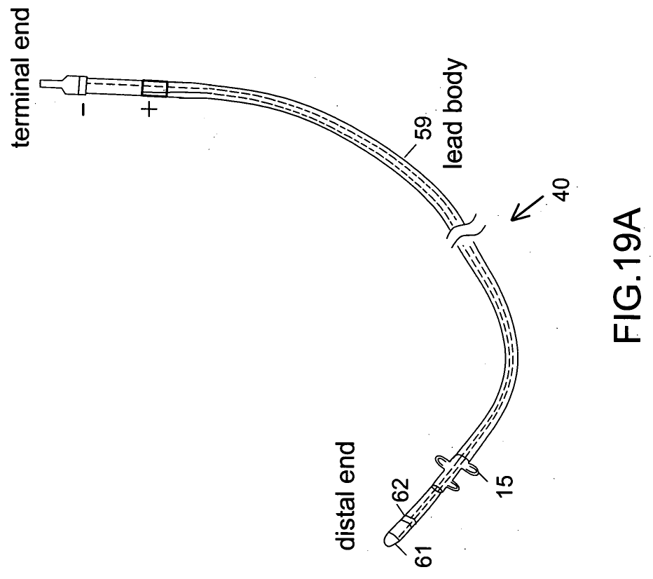

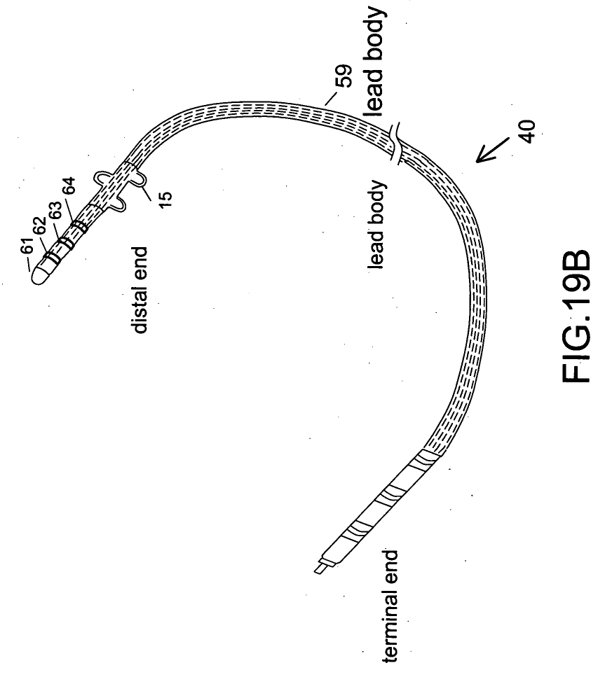

Referring now to FIGS. 19A and 19B, the implanted lead component of the system is somewhat similar to cardiac pacemaker leads, except for distal portion 40 (or electrode end) of the lead. The lead terminal preferably is linear, even though it can be bifurcated, and plug(s) into the cavity of the pulse generator means. The lead body 59 insulation may be constructed of medical grade silicone, silicone reinforced with polytetrafluoro-ethylene (PTFE), or polyurethane. The electrodes 61,62,63,64 for stimulating the sacral nerves 54 are typically implanted adjacent to the nerve tissue to be stimulated.

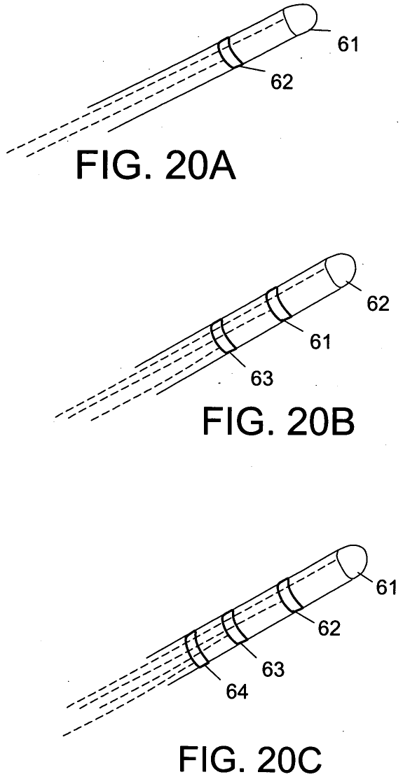

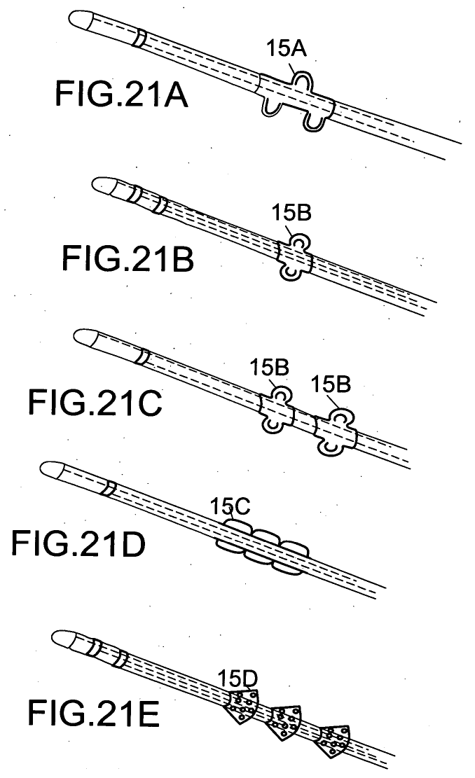

FIG. 20 shows close-up of the distal end of the lead. FIGS. 21A-21E show different types of anchoring sleeves pulled back form the most proximal electrode. All anchoring devices are made of silicone in this embodiment, even though they can be made of other bicompatible material. FIGS. 21A, 21B, and 21C show anchoring sleeves which have holes for suturing the lead to the tissue. FIG. 21D shows a type of suture sleeve that has grooves 15B for suturing to the tissue. FIG. 21E shows a passive fixation anchoring sleeve 15C where the holes in the silicone material promote tissue in-growth over time, for lead fixation.

The stimulating electrodes may be made of pure platinum, platinum/Iridium alloy or platinum/iridium coated with titanium nitride. The conductor connecting the terminal to the electrodes 61, 62, 63, 64 is made of an alloy of nickel-cobalt. The implanted lead design variables are also summarized in table three below.

TABLE 3
Lead design variables

| Proximal | | | Distal | | |
|---|---|---|---|---|---|
| End | | | End | | |
| | | | Conductor | | |
| | | | (connect- | | |
| | | | ing | | |
| | Lead body- | | proximal | Elec- | Elec- |
| Lead | Insulation | Lead- | and distal | trode - | trode - |
| Terminal | Materials | Coating | ends) | Material | Type |
| Linear | Polyure- | Anti- | Alloy of | Pure | Standard |
| bipolar | thane | microbial | Nickel- | Platinum | Ball and |
| | | coating | Cobalt | | Ring |
| | | | | | elec- |
| | | | | | trodes |
| Bifur- | Silicone | Anti- | | Platinum- | Steroid |
| cated | | Inflam- | | Iridium | eluting |
| | | matory | | (Pt/Ir) | |
| | | coating | | Alloy | |
| | Silicone | Lubricious | | Pt/Ir | |
| | with | coating | | coated | |
| | Polytetra- | | | with | |
| | fluoro- | | | Titanium | |
| | ethylene | | | Nitride | |
| | (PTFE) | | | | |
| | | | | Carbon | |

Once the lead is fabricated, coating such as anti-microbial, anti-inflammatory, or lubricious coating may be applied to the body of the lead.

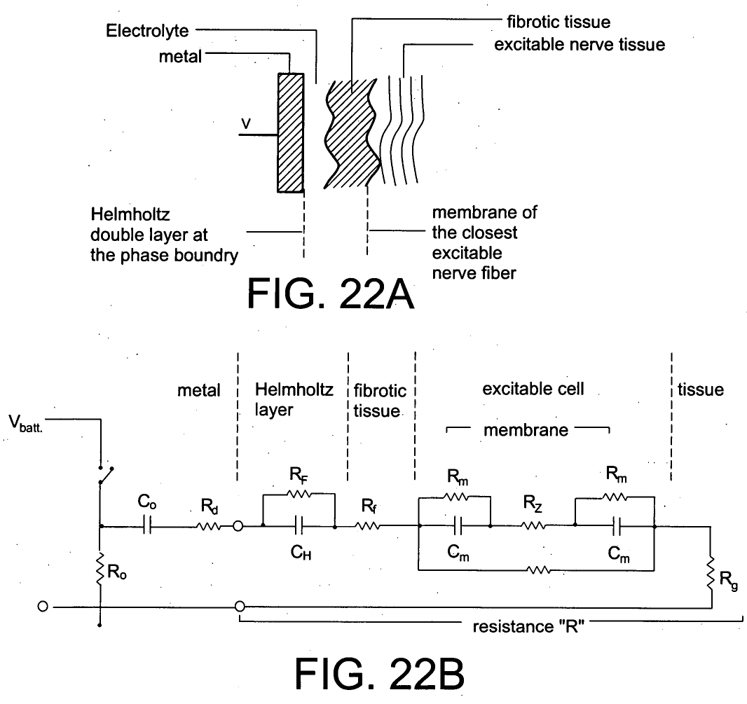

FIG. 22A summarizes electrode-tissue interface between the nerve tissue and electrodes 61, 62. There is a thin layer of fibrotic tissue between the stimulating electrode 61 and the excitable nerve fibers of the sacral nerves 54. FIG. 22B summarizes the most important properties of the metal/tissue phase boundary in an equivalent circuit diagram. Both the membrane of the nerve fibers and the electrode surface are represented by parallel capacitance and resistance. Application of a constant battery voltage Vbat from the pulse generator, produces voltage changes and current flow, the time course of which is crucially determined by the capacitive components in the equivalent circuit diagram. During the pulse, the capacitors Co, Ch and Cm are charged through the ohmic resistances, and when the voltage Vbat is turned off, the capacitors discharge with current flow on the opposite direction.

### Implanted Stimulus-Receiver Comprising a High Value Capacitor for Storing Charge, Used in Conjunction with an External Stimulator

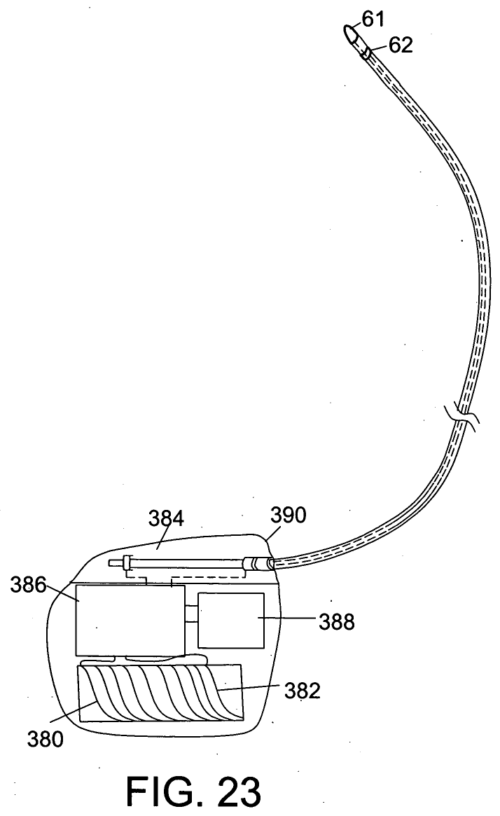

In one embodiment, the implanted stimulus-receiver may be a system which is RF coupled combined with a power source. In this embodiment, the implanted stimulus-receiver contains high value, small sized capacitor(s) for storing charge and delivering electric stimulation pulses for up to several hours by itself, once the capacitors are charged. The packaging is shown in FIG. 23. Using mostly hybrid components and appropriate packaging, the implanted portion of the system described below is conducive to miniaturization. As shown in FIG. 23, a solenoid coil 382 wrapped around a ferrite core 380 is used as the secondary of an air-gap transformer for receiving power and data to the implanted device. The primary coil is external to the body. Since the coupling between the external transmitter coil and receiver coil 382 may be weak, a high-efficiency transmitter/amplifier is used in order to supply enough power to the receiver coil 382. Class-D or Class-E power amplifiers may be used for this purpose. The coil for the external transmitter (primary coil) may be placed in the pocket of a customized garment.

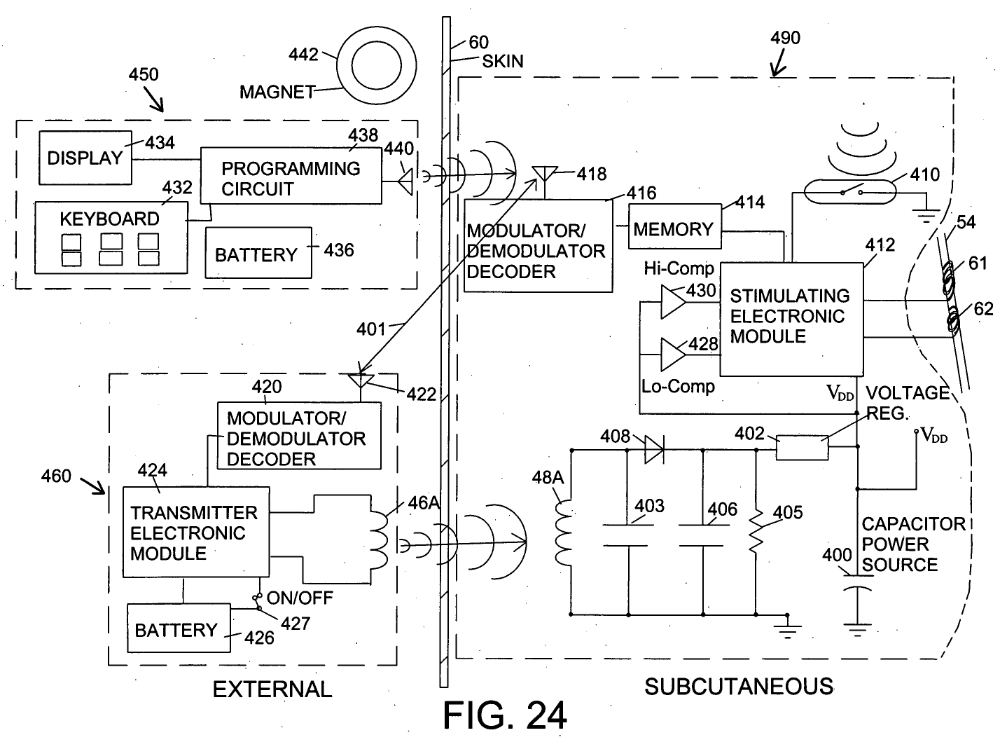

As shown in conjunction with FIG. 24 of the implanted stimulus-receiver 490 and the system, the receiving inductor 48A and tuning capacitor 403 are tuned to the frequency of the transmitter. The diode 408 rectifies the AC signals, and a small sized capacitor 406 is utilized for smoothing the input voltage V_{I} fed into the voltage regulator 402. The output voltage V_{D} of regulator 402 is applied to capacitive energy power supply and source 400 which establishes source power V_{DD}. Capacitor 400 is a big value, small sized capacative energy source which is classified as low internal impedance, low power loss and high charge rate capacitor, such as Panasonic Model No. 641 (available from Pansonic corporation).

The refresh-recharge transmitter unit 460 includes a primary battery 426, an ON/Off switch 427, a transmitter electronic module 442, an RF inductor power coil 46A, a modulator/demodulator 420 and an antenna 422.

When the ON/OFF switch is on, the primary coil 46A is placed in close proximity to skin 60 and secondary coil 48A of the implanted stimulator 490. The inductor coil 46A emits RF waves establishing EMF wave fronts which are received by secondary inductor 48A. Further, transmitter electronic module 424 sends out command signals which are converted by modulator/demodulator decoder 420 and sent via antenna 422 to antenna 418 in the implanted stimulator 490. These received command signals are demodulated by decoder 416 and replied and responded to, based on a program in memory 414 (matched against a “command table” in the memory). Memory 414 then activates the proper controls and the inductor receiver coil 48A accepts the RF coupled power from inductor 46A.

The RF coupled power, which is alternating or AC in nature, is converted by the rectifier 408 into a high DC voltage. Small value capacitor 406 operates to filter and level this high DC voltage at a certain level. Voltage regulator 402 converts the high DC voltage to a lower precise DC voltage while capacitive power source 400 refreshes and replenishes.

When the voltage in capacative source 400 reaches a predetermined level (that is V_{DD} reaches a certain predetermined high level), the high threshold comparator 430 fires and stimulating electronic module 412 sends an appropriate command signal to modulator/decoder 416. Modulator/decoder 416 then sends an appropriate “fully charged” signal indicating that capacitive power source 400 is fully charged, is received by antenna 422 in the refresh-recharge transmitter unit 460.

In one mode of operation, the patient may start or stop stimulation by waving the magnet 442 once near the implant. The magnet emits a magnetic force Lₘ which pulls reed switch 410 closed. Upon closure of reed switch 410, stimulating electronic module 412 in conjunction with memory 414 begins the delivery (or cessation as the case may be) of controlled electronic stimulation pulses to the sacral nerves 54 (sacral plexus) via a pair of electrodes. In another mode (AUTO), the stimulation is automatically delivered to the implanted lead based upon programmed ON/OFF times.

The programmer unit 450 includes keyboard 432, programming circuit 438, rechargeable battery 436, and display 434. The physician or medical technician programs programming unit 450 via keyboard 432. This program regarding the frequency, pulse width, modulation program, ON time etc. is stored in programming circuit 438. The programming unit 450 must be placed relatively close to the implanted stimulator 490 in order to transfer the commands and programming information from antenna 440 to antenna 418. Upon receipt of this programming data, modulator/demodulator and decoder 416 decodes and conditions these signals, and the digital programming information is captured by memory 414. This digital programming information is further processed by stimulating electronic module 412. In the DEMAND operating mode, after programming the implanted stimulator, the patient turns ON and OFF the implanted stimulator via hand held magnet 442 and the reed switch 410. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

### Programmer-Less Implantable Pulse Generator (IPG)

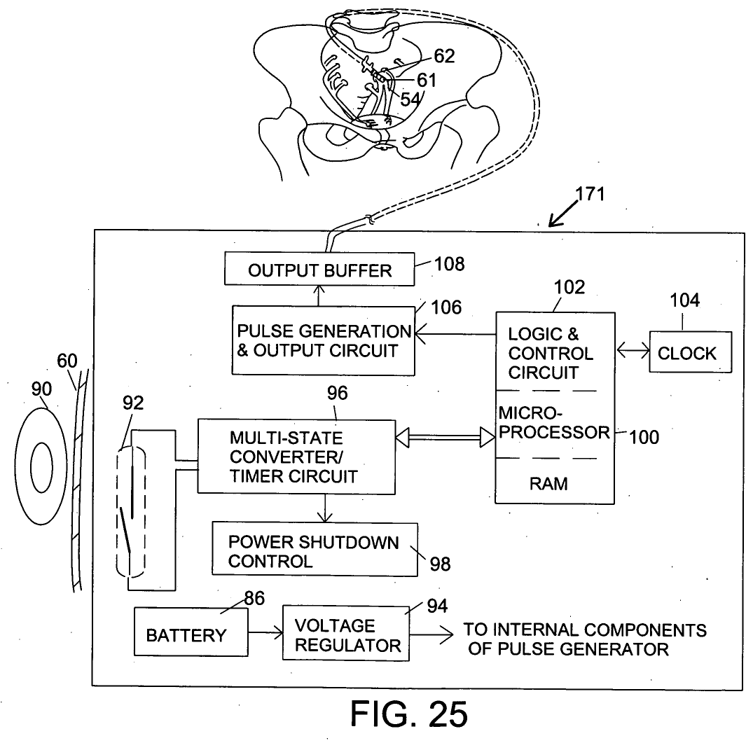

In one embodiment, a programmer-less implantable pulse generator (IPG) may be used. In this embodiment, shown in conjunction with FIG. 25, the implantable pulse generator 171 is provided with a reed switch 92 and memory circuitry 102. The reed switch 92 being remotely actuable by means of a magnet 90 brought into proximity of the pulse generator 171, in accordance with common practice in the art. In this embodiment, the reed switch 92 is coupled to a multi-state converter/timer circuit 96, such that a single short closure of the reed switch can be used as a means for non-invasive encoding and programming of the pulse generator 171 parameters.

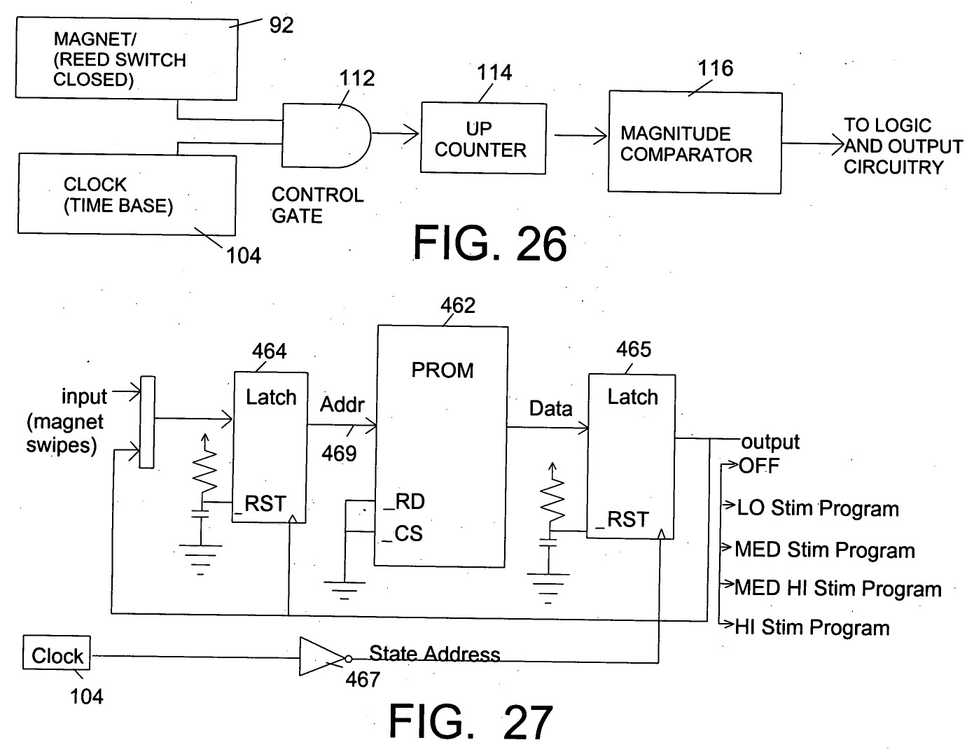

In one embodiment, shown in conjunction with FIG. 26, the closing of the reed switch 92 triggers a counter. The magnet 90 and timer are ANDed together. The system is configured such that during the time that the magnet 82 is held over the pulse generator 171, the output level goes from LOW stimulation state to the next higher stimulation state every 5 seconds. Once the magnet 82 is removed, regardless of the state of stimulation, an application of the magnet, without holding it over the pulse generator 171, triggers the OFF state, which also resets the counter.

Once the prepackaged/predetermined logic state is activated by the logic and control circuit 102, as shown in FIG. 25, the pulse generation and amplification circuit 106 deliver the appropriate electrical pulses to the sacral nerves 54 of the patient via an output buffer 108. The delivery of output pulses is configured such that the distal electrode 61 is the cathode and the proximal electrode 62 is the anode. Timing signals for the logic and control circuit 102 of the pulse generator 171 are provided by a crystal oscillator 104. The battery 86 of the pulse generator 171 has terminals connected to the input of a voltage regulator 94. The regulator 94 smoothes the battery output and supplies power to the internal components of the pulse generator 171. A microprocessor 100 controls the program parameters of the device, such as the voltage, pulse width, frequency of pulses, on-time and off-time. The microprocessor may be a commercially available, general purpose microprocessor or microcontroller, or may be a custom integrated circuit device augmented by standard RAM/ROM components.

In one embodiment, there are four stimulation states. A larger (or smaller) number of states can be achieved using the same methodology, and such is considered within the scope of the invention. These four states are, LOW stimulation state, LOW-MED stimulation state, MED stimulation state, and HIGH stimulation state. Examples of stimulation parameters (delivered to the sacral plexus) for each state are as follows,

LOW stimulation state example is,

Current output: 0.75 milliAmps.

Pulse width: 0.20 msec.

Pulse frequency: 20 Hz

ON for 5 minutes

LOW-MED stimulation state example is,

Current output: 1.5 milliAmps,

Pulse width: 0.30 msec.

Pulse frequency: 22 Hz

ON for 7.5 minutes

MED stimulation state example is,

Current output: 2.0 milliAmps.

Pulse width: 0.40 msec.

Pulse frequency: 25 Hz

ON for 15 minutes

HIGH stimulation state example is,

Current output: 3.0 milliAmps,

Pulse width: 0.50 msec.

Pulse frequency: 30 Hz

ON for 30 minutes

These prepackaged/predetermined programs are mearly examples, and the actual stimulation parameters will deviate from these depending on the treatment application.

It will be readily apparent to one skilled in the art, that other schemes can be used for the same purpose. For example, instead of placing the magnet 90 on the pulse generator 171 for a prolonged period of time, different stimulation states can be encoded by the sequence of magnet applications. Accordingly, in an alternative embodiment there can be three logic states, OFF, LOW stimulation (LS) state, and HIGH stimulation (HS) state. Each logic state again corresponds to a prepackaged/predetermined program such as presented above. In such an embodiment, the system could be configured such that one application of the magnet triggers the generator into LS State. If the generator is already in the LS state then one application triggers the device into OFF State. Two successive magnet applications triggers the generator into MED stimulation state, and three successive magnet applications triggers the pulse generator in the HIGH Stimulation State. Subsequently, one application of the magnet while the device is in any stimulation state, triggers the device OFF.

FIG. 27 shows a representative digital circuitry used for the basic state machine circuit. The circuit consists of a PROM 462 that has part of its data fed back as a state address. Other address lines 469 are used as circuit inputs, and the state machine changes its state address on the basis of these inputs. The clock 104 is used to pass the new address to the PROM 462 and then pass the output from the PROM 462 to the outputs and input state circuits. The two latches 464, 465 are operated 180° out of phase to prevent glitches from unexpectedly affecting any output circuits when the ROM changes state. Each state responds differently according to the inputs it receives.

The advantage of this embodiment is that it is cheaper to manufacture than a fully programmable implantable pulse generator (IPG).

### Programmable Implantable Pulse Generator (IPG)

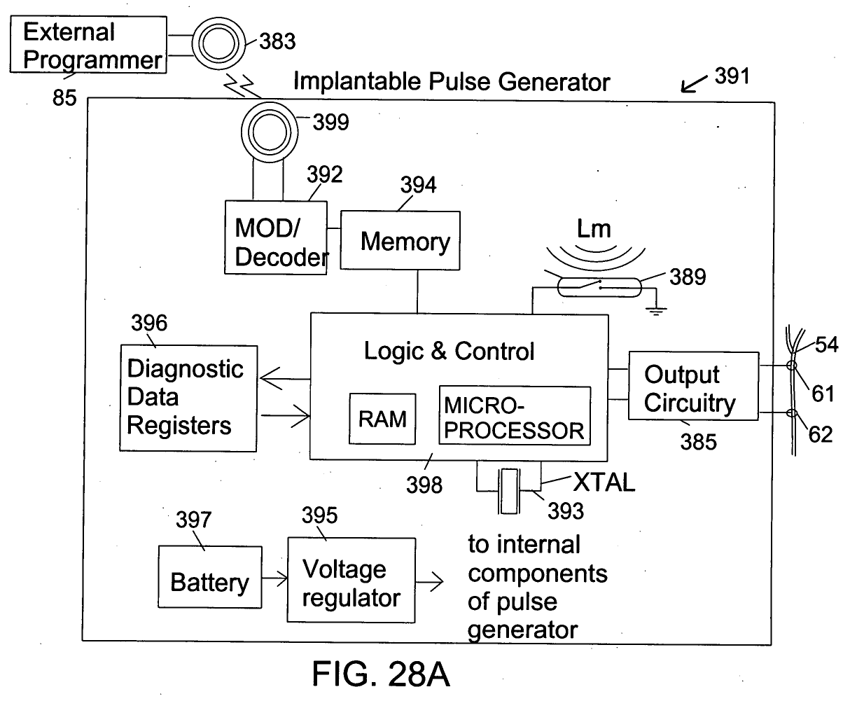

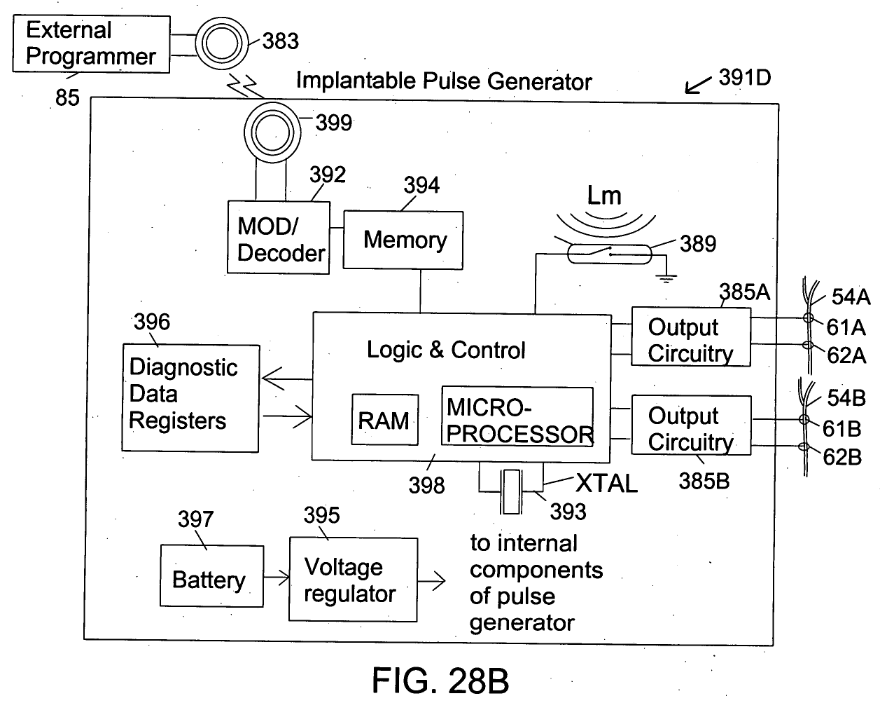

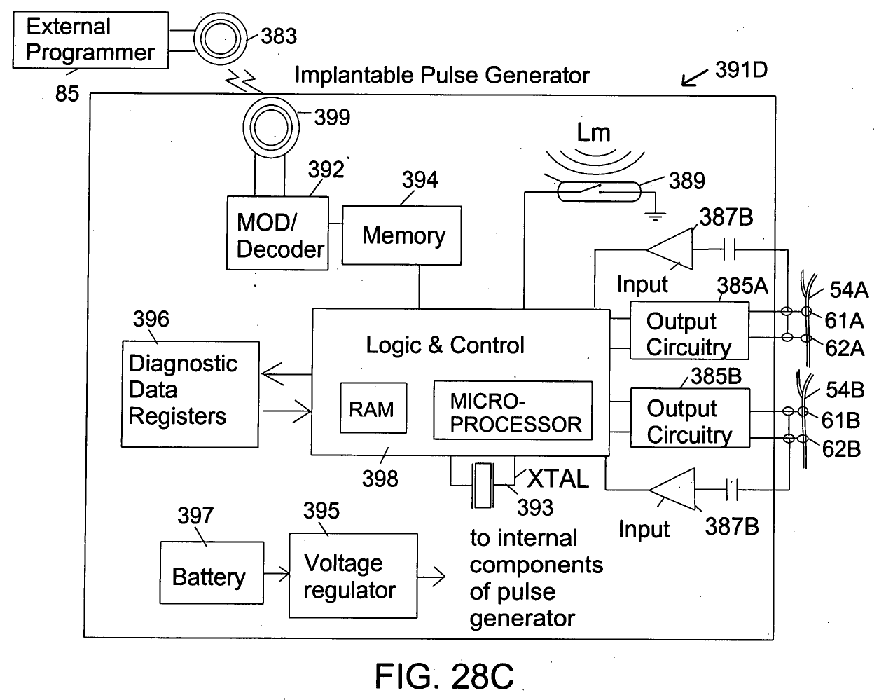

In one embodiment, a fully programmable implantable pulse generator (IPG), capable of generating stimulation and blocking pulses may be used. Shown in conjunction with FIG. 28A, the implantable pulse generator unit 391 is preferably a microprocessor based device, where the entire circuitry is encased in a hermetically sealed titanium can. As shown in the overall block diagram, the logic & control unit 398 provides the proper timing for the output circuitry 385 to generate electrical pulses that are delivered to electrodes 61, 62 via a lead 40. Programming of the implantable pulse generator (IPG) is done via an external programmer 85, as described later. Once activated or programmed via an external programmer 85, the implanted pulse generator 391 provides appropriate electrical stimulation pulses to the sacral nerve(s) 54 via electrodes 61,62.

As was previously mentioned in the background section, the stimulation to sacral and/or pudendal nerve(s) may be unilateral or bilateral. For bilateral stimulation, a dual channel stimulator with two leads may be utilized. This is shown in conjunction with FIGS. 28B and 28C.

In one embodiment, as shown in conjunction with FIG. 28B, the pulse generator is adapted to be used as a dual channel stimulator, wherein the header of the implantable pulse generator 391D is adapted to accommodate two leads. The functioning of the circuitry is similar to as described for FIG. 28A, except the logic and control unit 398, controls the output of 2 channels. The two channels may be operated synchronous to each other or independent of each other, depending on physician judgement for the particular patient. In another embodiment, as shown in conjunction with FIG. 28C, sensing from tissues may be utilized for a closed-loop system. As shown in FIG. 28C, sense amplifier(s) 387A and 387B can be used in conjunction with the same two leads used for delivering output pulses.

This embodiment also comprises predetermined/pre-packaged programs. Examples of four stimulation states were given in the previous section, under “Programmer-less Implantable Pulse Generator (IPG)”. These predetermined/pre-packaged programs comprise unique combinations of pulse amplitude, pulse width, pulse morphology, pulse frequency, ON-time and OFF-time. Any number of predetermined/pre-packaged programs, even 100, can be stored in the memory of the implantable pulse generator of this invention, and are considered within the scope of the invention.

Examples of additional predetermined/pre-packaged programs for urological disorders are:

### Program One

Pulse amplitude: 0.5 volts

Pulse width: 0.150 msec.

Pulse frequency: 5 Hz

Cycles: 10 seconds ON-time and 10 seconds OFF-time in repeating cycles.

Configuration: Unipolar

### Program Two

Pulse amplitude: 0.75 volts

Pulse width: 0.160 msec.

Pulse frequency: 7 Hz

Cycles: 12 seconds ON-time and 8 seconds OFF-time in repeating cycles.

Configuration: Unipolar

### Program Three

Pulse amplitude: 1.0 volts

Pulse width: 0.175 msec.

Pulse frequency: 9 Hz

Cycles: 12 seconds ON-time and 6 seconds OFF-time in repeating cycles.

Configuration: Unipolar

### Program Four

Pulse amplitude: 1.5 volts

Pulse width: 0.200 msec.

Pulse frequency: 10 Hz

Cycles: 12 seconds ON-time and 6 seconds OFF-time in repeating cycles.

Configuration: Unipolar

### Program Five

Pulse amplitude: 2.0 volts

Pulse width: 0.225 msec.

Pulse frequency: 10 Hz

Cycles: 12 seconds ON-time and 5 seconds OFF-time in repeating cycles.

Configuration: Unipolar

### Program Six

Pulse amplitude: 2.5 volts

Pulse width: 0.250 msec.

Pulse frequency: 15 Hz

Cycles: 12 seconds ON-time and 4 seconds OFF-time in repeating cycles.

Configuration: Unipolar

### Program Seven

Pulse amplitude: 3.5 volts

Pulse width: 0.250 msec.

Pulse frequency: 20 Hz

Cycles: 12 seconds ON-time and 4 seconds OFF-time in repeating cycles.

Configuration: Unipolar

### Program Eight

Pulse amplitude: 4.5 volts

Pulse width: 0.250 msec.

Pulse frequency: 20 Hz.

Cycles: 15 seconds ON-time and 3 seconds OFF-time in repeating cycles.

Configuration: Unipolar

### Program Nine

Pulse amplitude: 2.0 volts

Pulse width: 0.225 msec.

Pulse frequency: 10 Hz

Cycles: 12 seconds ON-time and 5 seconds OFF-time in repeating cycles.

Configuration: Bipolar

### Program Ten

Pulse amplitude: 2.5 volts

Pulse width: 0.250 msec.

Pulse frequency: 15 Hz

Cycles: 12 seconds ON-time and 4 seconds OFF-time in repeating cycles.

Configuration: Bipolar

### Program Eleven (Complex Pulses)

Pulse amplitude: 1.5 volts

Pulse width: 0.20 msec.

Pulse frequency: 10 Hz

Pulse type: step pulses

Cycles: 10 seconds ON-time and 5 seconds OFF-time in repeating cycles.

Configuration: unipolar

### Program Twelve (Complex Pulses)

Pulse amplitude: 1.5 volts

Pulse width: 0.20 msec.

Pulse frequency: 12 Hz

Pulse type: step pulses

Cycles: 10 seconds ON-time and 5 seconds OFF-time in repeating cycles.

Configuration: bipolar

### Pre-Packaged Programs for Fecal Incontinence

### Program One

Pulse amplitude: 1.5 volts

Pulse width: 0.20 msec.

Pulse frequency: 12 Hz

Cycles: 6 seconds ON-time and 2 seconds OFF-time in repeating cycles.

### Program Two

Pulse amplitude: 2.0 volts

Pulse width: 0.225 msec.

Pulse frequency: 15 Hz

Cycles: 5 seconds ON-time and 1 second OFF-time in repeating cycles.

### Program Three

Pulse amplitude: 2.5 volts

Pulse width: 0.250 msec.

Pulse frequency: 15 Hz

Cycles: 6 seconds ON-time and 1 second OFF-time in repeating cycles.

### Program Four

Pulse amplitude: 4.0 volts

Pulse width: 0.225 msec.

Pulse frequency: 18 Hz

Cycles: 10 seconds ON-time and 1 second OFF-time in repeating cycles.

### Program Five

Pulse amplitude: 6.0 volts

Pulse width: 0.250 msec.

Pulse frequency: 18 Hz

Cycles: 6 seconds ON-time and 2 seconds OFF-time in repeating cycles.

These prepackaged/predetermined programs are mearly examples, and the actual stimulation parameters will deviate from these depending on the treatment application and physician preference. One advantage of predetermined/pre-packaged program is that it can be readily activated by a program number. A simple version of a programmer, adapted to activate only a limited number of predetermined/pre-packaged programs may also be supplied to the patient.

In addition, each variable parameter may be individually adjusted and stored in the memory 394. The range of programmable electrical stimulation parameters include both stimulating and blocking frequencies, and are shown in table four below.

TABLE 4
Programmable electrical parameter range

| PARAMER | RANGE |
|---|---|
| Pulse Amplitude | 0.1 Volt-25 Volts |
| Pulse width | 20 μS-5 mSec. |
| Stim. Frequency | 2 Hz-80 Hz |
| Freq. for blocking | DC to 750 Hz |
| On-time | 3 Secs-24 hours |
| Off-time | 1 Sec.-24 hours |
| Ramp | ON/OFF |
| Mode | Unipolar, Bipolar |

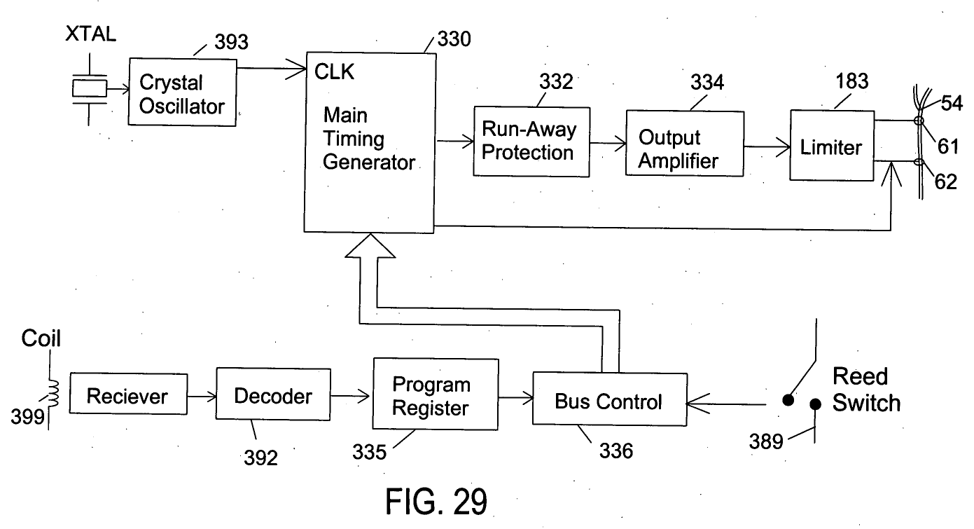

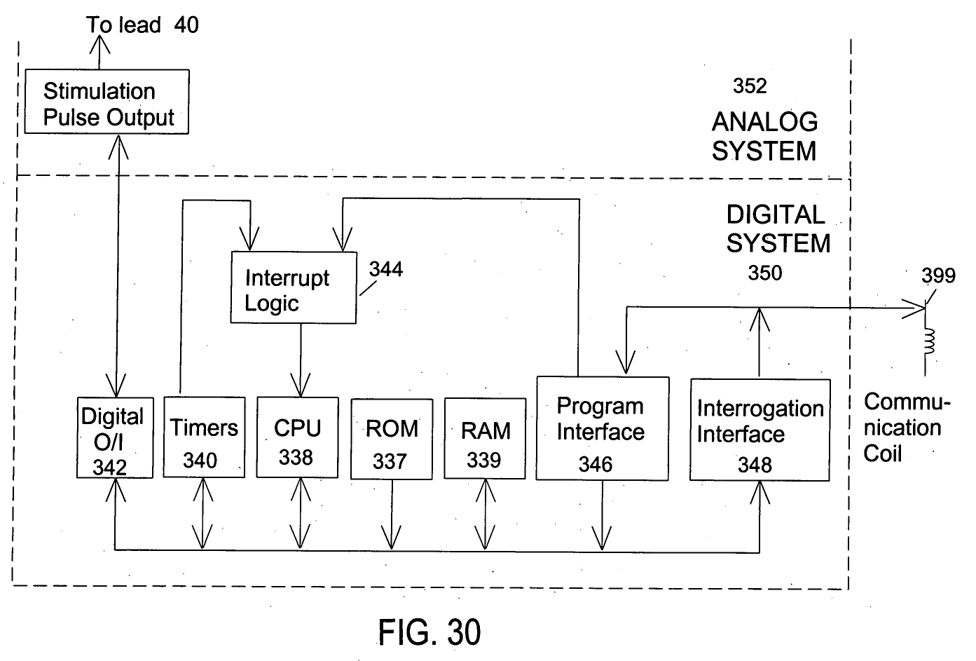

Shown in conjunction with FIGS. 29 and 30, the electronic stimulation module comprises both digital 350 and analog 352 circuits. A main timing generator 330 (shown in FIG. 29), controls the timing of the analog output circuitry for delivering neuromodulating pulses to the sacral nerve(s) 54, via output amplifier 334. Limiter 183 prevents excessive stimulation energy from getting into the sacral nerve(s) 54. The main timing generator 330 receiving clock pulses from crystal oscillator 393. Main timing generator 330 also receiving input from programmer 85 via coil 399. FIG. 30 highlights other portions of the digital system such as CPU 338, ROM 337, RAM 339, program interface 346, interrogation interface 348, timers 340, and digital O/I 342.

Most of the digital functional circuitry 350 is on a single chip (IC). This monolithic chip along with other IC's and components such as capacitors and the input protection diodes are assembled together on a hybrid circuit. As well known in the art, hybrid technology is used to establish the connections between the circuit and the other passive components. The integrated circuit is hermetically encapsulated in a chip carrier. A coil 399 situated under the hybrid substrate is used for bidirectional telemetry. The hybrid and battery 397 are encased in a titanium can 65. This housing is a two-part titanium capsule that is hermetically sealed by laser welding. Alternatively, electron-beam welding can also be used. The header 79 is a cast epoxy-resin with hermetically sealed feed-through, and form the lead 40 connection block.

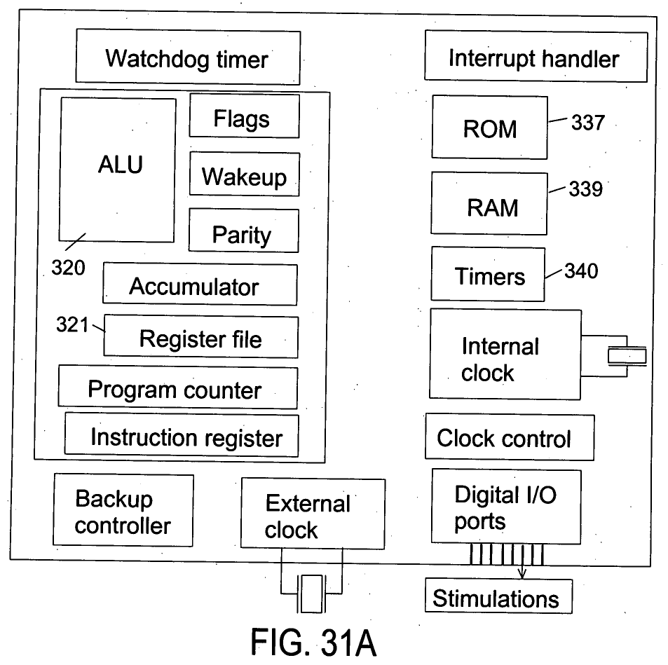

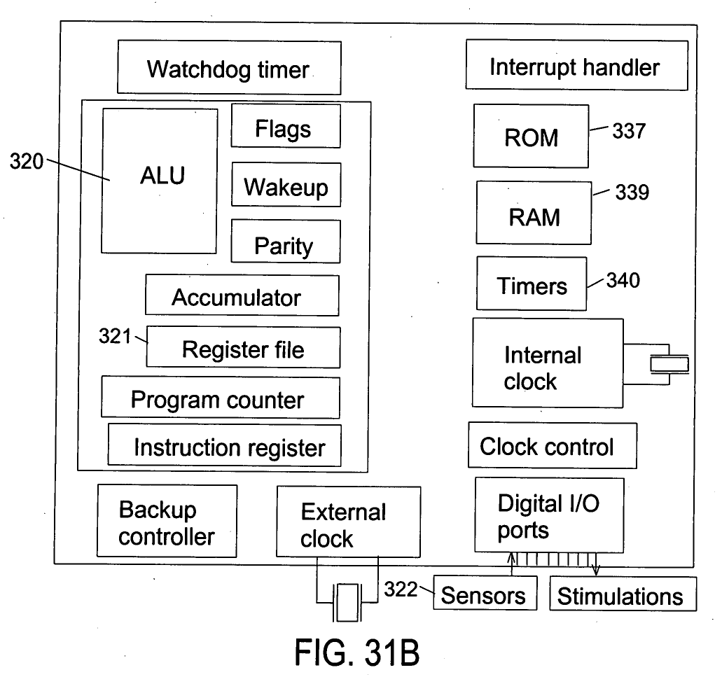

For further details, FIG. 31A highlights the general components of an 8-bit microprocessor as an example. It will be obvious to one skilled in the art that higher level microprocessor, such as a 16-bit or 32-bit may be utilized, and is considered within the scope of this invention. It comprises a ROM 337 to store the instructions of the program to be executed and various programmable parameters, a RAM 339 to store the various intermediate parameters, timers 340 to track the elapsed intervals, a register file 321 to hold intermediate values, an ALU 320 to perform the arithmetic calculation, and other auxiliary units that enhance the performance of a microprocessor-based IPG system.

The size of ROM 337 and RAM 339 units are selected based on the requirements of the algorithms and the parameters to be stored. The number of registers in the register file 321 are decided based upon the complexity of computation and the required number of intermediate values. Timers 340 of different precision are used to measure the elapsed intervals. Even though this embodiment does not have external sensors to control timing, future embodiments may have sensors 322 to effect the timing as shown in conjunction with FIG. 31B.

In this embodiment, the two main components of microprocessor are the datapath and control. The datapath performs the arithmetic operation and the control directs the datapath, memory, and I/O devices to execute the instruction of the program. The hardware components of the microprocessor are designed to execute a set of simple instructions. In general the complexity of the instruction set determines the complexity of datapth elements and controls of the microprocessor.

In this embodiment, the microprocessor is provided with a fixed operating routine. Future embodiments may be provided with the capability of actually introducing program changes in the implanted pulse generator. The instruction set of the microprocessor, the size of the register files, RAM and ROM are selected based on the performance needed and the type of the algorithms used. In this application of pulse generator, in which several algorithms can be loaded and modified, Reduced Instruction Set Computer (RISC) architecture is useful. RISC architecture offers advantages because it can be optimized to reduce the instruction cycle which in turn reduces the run time of the program and hence the current drain. The simple instruction set architecture of RISC and its simple hardware can be used to implement any algorithm without much difficulty. Since size is also a major consideration, an 8-bit microprocessor is used for the purpose. As most of the arithmetic calculation are based on a few parameters and are rather simple, an accumulator architecture is used to save bits from specifying registers. Each instruction is executed in multiple clock cycles, and the clock cycles are broadly classified into five stages: an instruction fetch, instruction decode, execution, memory reference, and write back stages. Depending on the type of the instruction, all or some of these stages are executed for proper completion.

Initially, an optimal instruction set architecture is selected based on the algorithm to be implemented and also taking into consideration the special needs of a microprocessor based implanted pulse generator (IPG). The instructions are broadly classified into Load/store instructions, Arithmetic and logic instructions (ALU), control instructions and special purpose instructions.

The instruction format is decided based upon the total number of instructions in the instruction set. The instructions fetched from memory are 8 bits long in this example. Each instruction has an opcode field (2 bits), a register specifier field (3-bits), and a 3-bit immediate field. The opcode field indicates the type of the instruction that was fetched. The register specifier indicates the address of the register in the register file on which the operations are performed. The immediate field is shifted and sign extended to obtain the address of the memory location in load/store instruction. Similarly, in branch and jump instruction, the offset field is used to calculate the address of the memory location the control needs to be transferred to.

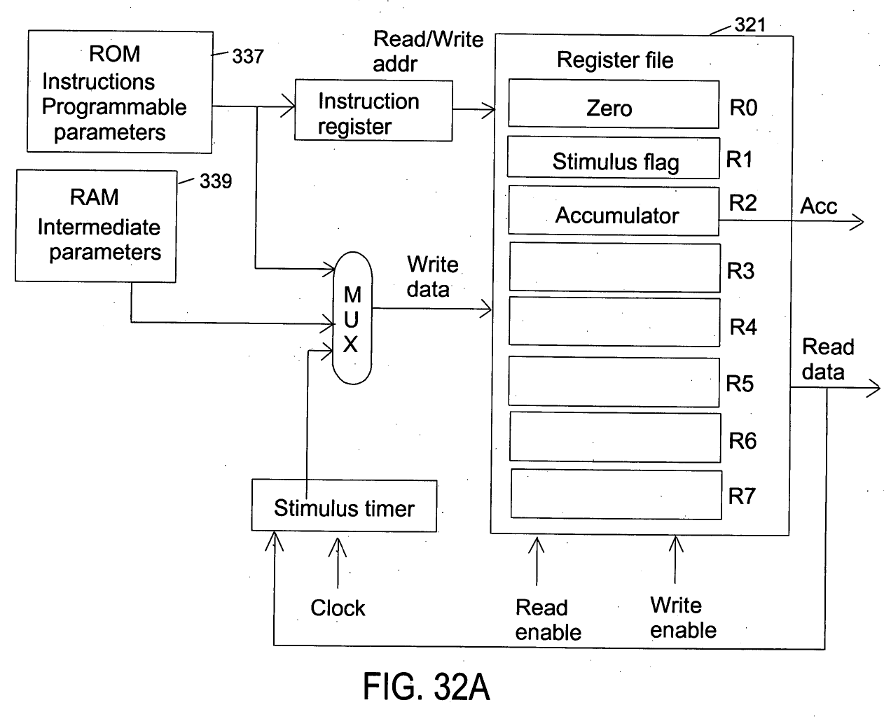

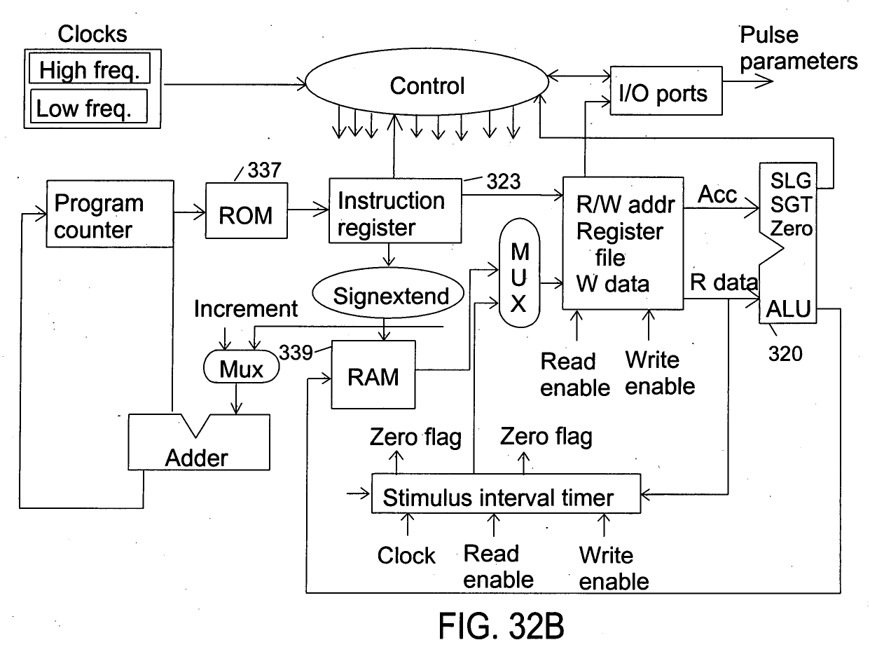

Shown in conjunction with FIG. 32A, the register file 321, which is a collection of registers in which any register can be read from or written to specifying the number of the register in the file. Based on the requirements of the design, the size of the register file is decided. For the purposes of implementation of stimulation pulses algorithms, a register file of eight registers is sufficient, with three special purpose register (0-2) and five general purpose registers (3-7), as shown in FIG. 32A. Register “0” always holds the value “zero”. Register “1” is dedicated to the pulse flags. Register “2” is an accumulator in which all the arithmetic calculations are performed. The read/write address port provides a 3-bit address to identify the register being read or written into. The write data port provides 8-bit data to be written into the registers either from ROM/RAM or timers. Read enable control, when asserted enables the register file to provide data at the read data port. Write enable control enables writing of data being provided at the write data port into a register specified by the read/write address.

Generally, two or more timers are required to implement the algorithm for the IPG. The timers are read and written into just as any other memory location. The timers are provided with read and write enable controls.

The arithmetic logic unit is an important component of the microprocessor. It performs the arithmetic operation such as addition, subtraction and logical operations such as AND and OR. The instruction format of ALU instructions consists of an opcode field (2 bits), a function field (2 bits) to indicate the function that needs to be performed, and a register specifier (3 bits) or an immediate field (4 bits) to provide an operand.

The hardware components discussed above constitute the important components of a datapath. Shown in conjunction with FIG. 32B, there are some special purpose registers such a program counter (PC) to hold the address of the instruction being fetched from ROM 337 and instruction register (IR) 323, to hold the instruction that is fetched for further decoding and execution. The program counter is incremented in each instruction fetch stage to fetch sequential instruction from memory. In the case of a branch or jump instruction, the PC multiplexer allows to choose from the incremented PC value or the branch or jump address calculated. The opcode of the instruction fetched (IR) is provided to the control unit to generate the appropriate sequence of control signals, enabling data flow through the datapath. The register specification field of the instruction is given as read/write address to the register file, which provides data from the specified field on the read data port. One port of the ALU is always provided with the contents of the accumulator and the other with the read data port. This design is therefore referred to as accumulator-based architecture. The sign-extended offset is used for address calculation in branch and jump instructions. The timers are used to measure the elapsed interval and are enabled to count down on a low-frequency clock. The timers are read and written into, just as any other memory location (FIG. 32B).

In a multicycle implementation, each stage of instruction execution takes one clock cycle. Since the datapath takes multiple clock cycles per instruction, the control must specify the signals to be asserted in each stage and also the next step in the sequence. This can be easily implemented as a finite state machine.

A finite state machine consists of a set of states and directions on how to change states. The directions are defined by a next-state function, which maps the current state and the inputs to a new state. Each stage also indicates the control signals that need to be asserted. Every state in the finite state machine takes one clock cycle. Since the instruction fetch and decode stages are common to all the instruction, the initial two states are common to all the instruction. After the execution of the last step, the finite state machine returns to the fetch state.

A finite state machine can be implemented with a register that holds the current stage and a block of combinational logic such as a PLA. It determines the datapath signals that need to be asserted as well as the next state. A PLA is described as an array of AND gates followed by an array of OR gates. Since any function can be computed in two levels of logic, the two-level logic of PLA is used for generating control signals.

The occurrence of a wakeup event initiates a stored operating routine corresponding to the event. In the time interval between a completed operating routine and a next wake up event, the internal logic components of the processor are deactivated and no energy is being expended in performing an operating routine.

A further reduction in the average operating current is obtained by providing a plurality of counting rates to minimize the number of state changes during counting cycles. Thus intervals which do not require great precision, may be timed using relatively low counting rates, and intervals requiring relatively high precision, such as stimulating pulse width, may be timed using relatively high counting rates.

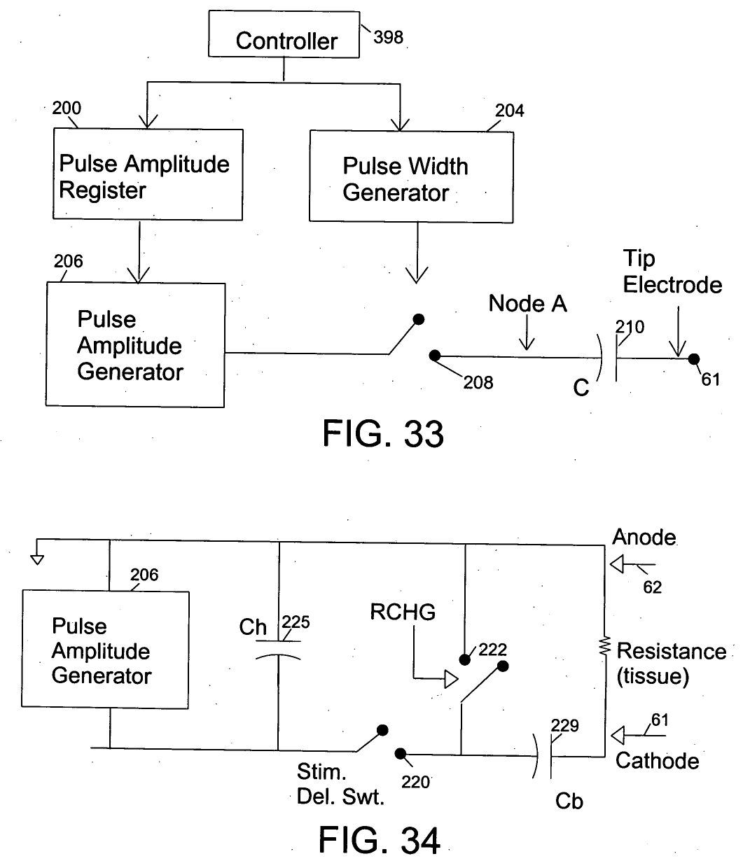

The logic and control unit 398 of the IPG controls the output amplifiers. The pulses have predetermined energy (pulse amplitude and pulse width) and are delivered at a time determined by the therapy stimulus controller. The circuitry in the output amplifier, shown in conjunction with (FIG. 33) generates an analog voltage or current that represents the pulse amplitude. The stimulation controller module initiates a stimulus pulse by closing a switch 208 that transmits the analog voltage or current pulse to the nerve tissue through the tip electrode 61 of the lead 40. The output circuit receiving instructions from the stimulus therapy controller 398 that regulates the timing of stimulus pulses and the amplitude and duration (pulse width) of the stimulus. The pulse amplitude generator 206 determines the configuration of charging and output capacitors necessary to generate the programmed stimulus amplitude. The output switch 208 is closed for a period of time that is controlled by the pulse width generator 204. When the output switch 208 is closed, a stimulus is delivered to the tip electrode 61 of the lead 40.

The constant-voltage output amplifier applies a voltage pulse to the distal electrode (cathode) 61 of the lead 40. A typical circuit diagram of a voltage output circuit is shown in FIG. 34. This configuration contains a stimulus amplitude generator 206 for generating an analog voltage. The analog voltage represents the stimulus amplitude and is stored on a holding capacitor Cₕ 225. Two switches are used to deliver the stimulus pulses to the lead 40, a stimulating delivery switch 220, and a recharge switch 222, that reestablishes the charge equilibrium after the stimulating pulse has been delivered to the nerve tissue. Since these switches have leakage currents that can cause direct current (DC) to flow into the lead system 40, a DC blocking capacitor C_{b} 229, is included. This is to prevent any possible corrosion that may result from the leakage of current in the lead 40. When the stimulus delivery switch 220 is closed, the pulse amplitude analog voltage stored in the (Cₕ 225) holding capacitor is transferred to the cathode electrode 61 of the lead 40 through the coupling capacitor, C_{b} 229. At the end of the stimulus pulse, the stimulus delivery switch 220 opens. The pulse duration being the interval from the closing of the switch 220 to its reopening. During the stimulus delivery, some of the charge stored on Cₕ 225 has been transferred to C_{b} 229, and some has been delivered to the lead system 40 to stimulate the nerve tissue.

To re-establish equilibrium, the recharge switch 222 is closed, and a rapid recharge pulse is delivered. This is intended to remove any residual charge remaining on the coupling capacitor C_{b} 229, and the stimulus electrodes on the lead (polarization). Thus, the stimulus is delivered as the result of closing and opening of the stimulus delivery 220 switch and the closing and opening of the RCHG switch 222. At this point, the charge on the holding Cₕ 225 must be replenished by the stimulus amplitude generator 206 before another stimulus pulse can be delivered.

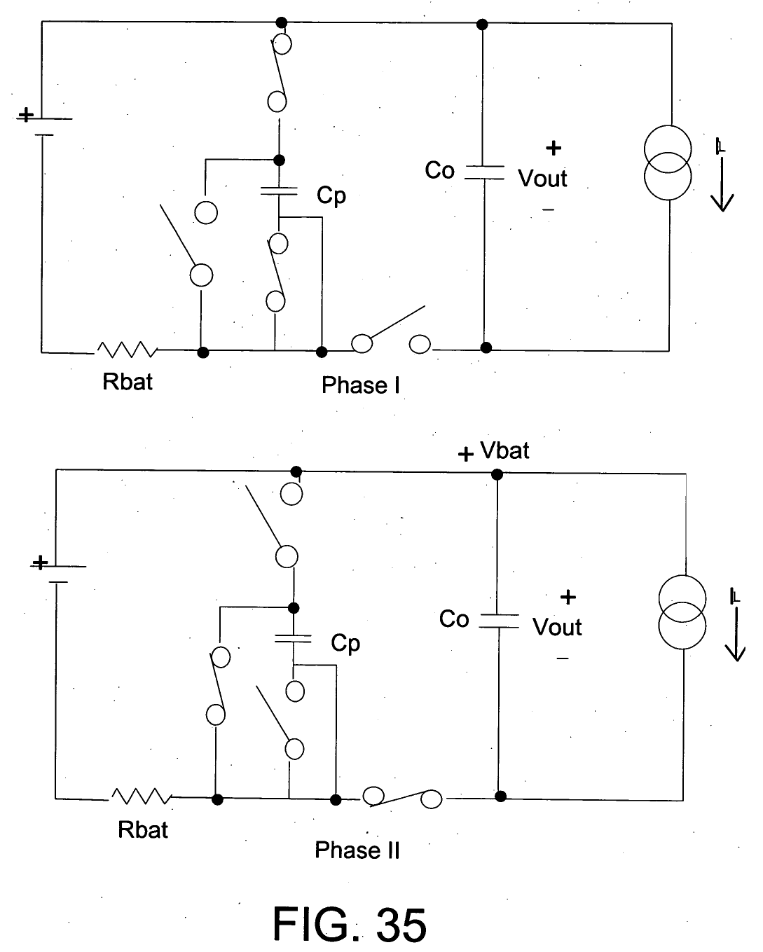

The pulse generating unit charges up a capacitor and the capacitor is discharged when the control (timing) circuitry requires the delivery of a pulse. This embodiment utilizes a constant voltage pulse generator, even though a constant current pulse generator can also be utilized. Pump-up capacitors are used to deliver pulses of larger magnitude than the potential of the batteries. The pump up capacitors are charged in parallel and discharged into the output capacitor in series. Shown in conjunction with FIG. 35 is a circuit diagram of a voltage doubler which is shown here as an example. For higher multiples of battery voltage, this doubling circuit can be cascaded with other doubling circuits. As shown in FIG. 35, during phase I (top of FIG. 35), the pump capacitor Cₚ is charged to V_{bat} and the output capacitor Cₒ supplies charge to the load. During phase II, the pump capacitor charges the output capacitor, which is still supplying the load current. In this case, the voltage drop across the output capacitor is twice the battery voltage.

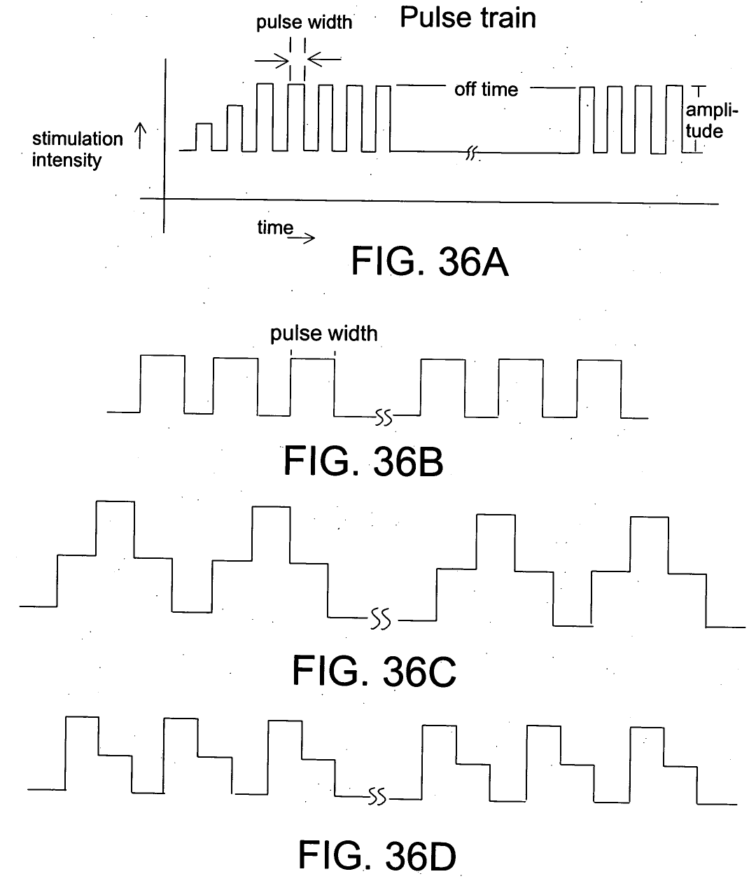

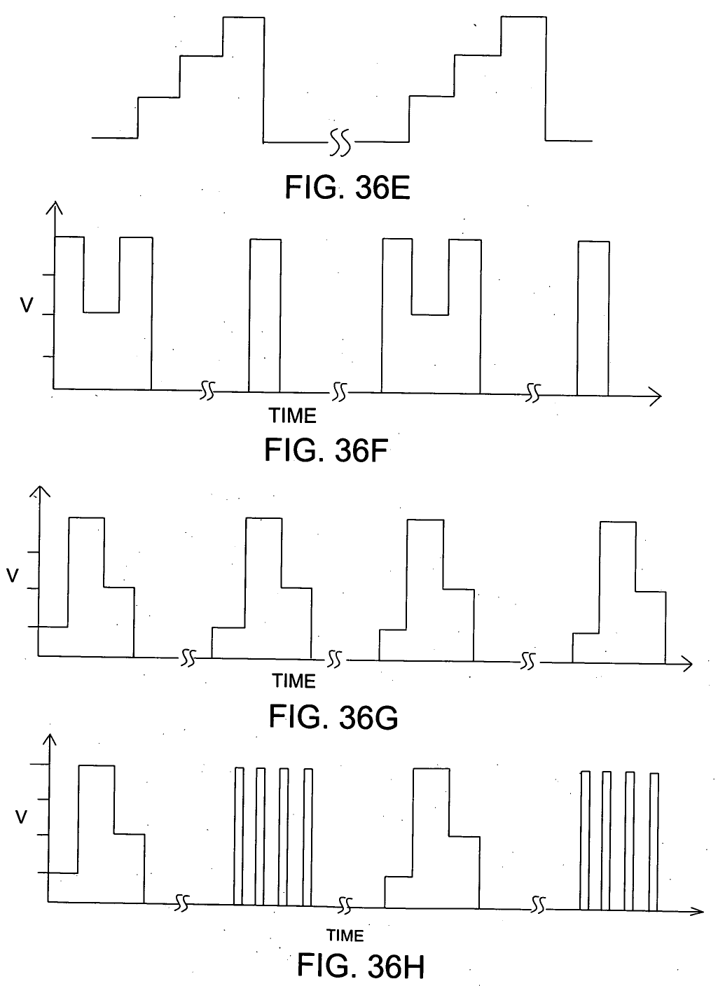

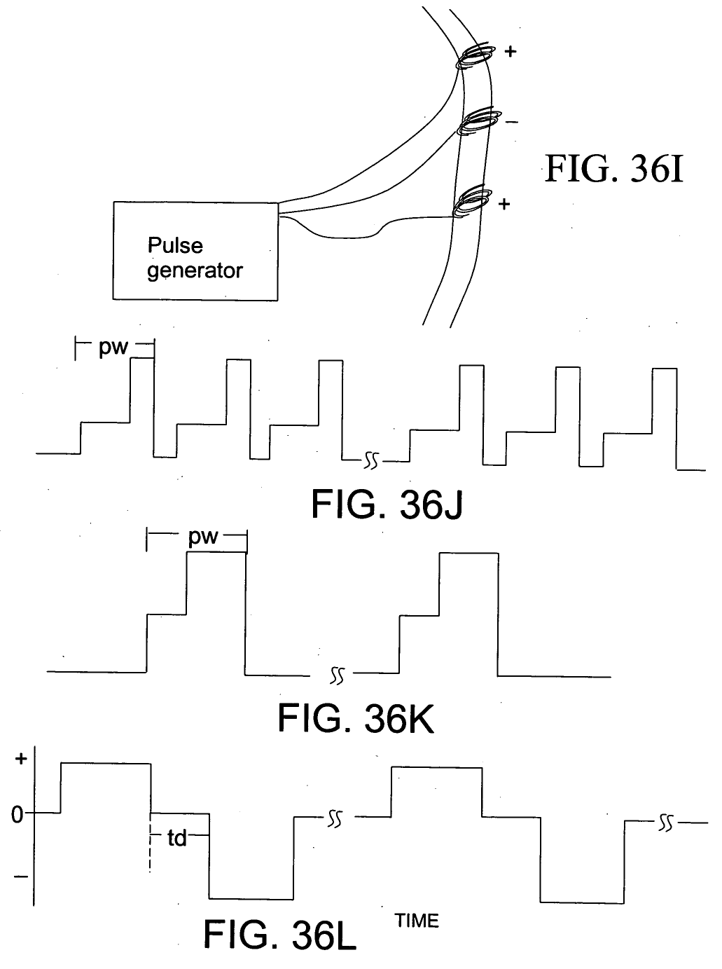

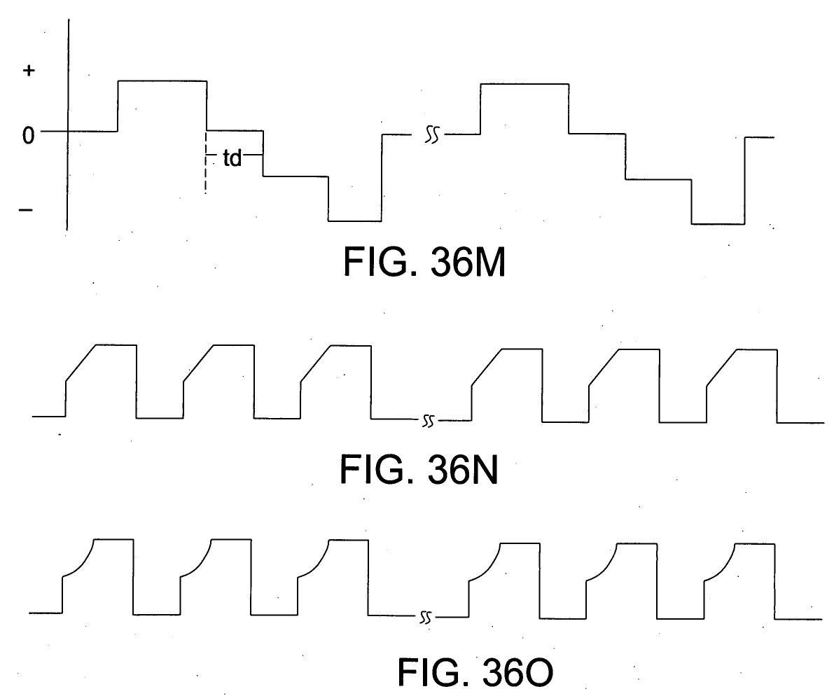

FIG. 36A shows one example of the pulse trains that may be delivered with this embodiment or in prior art sacral nerve(s) stimulators. The microcontroller is configured to deliver the pulse train as shown in the figure, i.e. there is “ramping up” of the pulse train. The purpose of the ramping-up is to avoid sudden changes in stimulation, when the pulse train begins. The ramping-up or ramping-down is optional, and may be programmed into the microcontroller.

The prior art systems delivering fixed rectangular pulses provide limited capability for selective stimulation or neuromodulation of sacral nerve(s). A fixed rectangular pulse, whether constant voltage or constant current, will recruit either i) A-fibers, or ii) A and B fibers, or iii) A and B and C fibers. Only one of these three discrete states can be achieved. This form of modulation is severely limited for providing therapy for neurological disorders.

In the method and system of the current invention, the microcontroller is configured to deliver rectangular and complex pulses. Complex pulses comprise non-rectangular, biphasic, multi-step, and other complex pulses where the amplitude is varying during the pulse. Advantageously, these complex pulses provide a new dimension to selective stimulation or neuromodulation of sacral nerve(s) to provide therapy for urological disorders, such as urinary incontinence/fecal incontinence.

Examples of these pulses and pulse trains are shown in FIGS. 36B to 36H. Selective stimulation with these complex pulses takes into account the threshold properties of different types of nerve fibers, as well as, the different refractory properties of different types of nerve fibers that are contained in the sacral nerve(s).

For example in the multi-step pulse shown in FIG. 36C, the first part of the pulse will tend to recruit large diameter (and myelinated) fibers, such as A and B fibers. The middle portion of the pulse where the amplitude is highest, will tend to recruit c-fibers which are the smallest fibers, and the last portion of the pulse will again tend to recruit the large diameter fibers provided they are not refractory. The multi-step (and multi-amplitude) pulses shown in FIG. 36E will tend to recruit large diameter fibers initially, and the later part of the pulse will tend to recruit the smaller diameter C-fibers.

Further, as shown in the examples of FIGS. 36F and 36H, complex and simple pulses, or pulse trains may be alternated. It will be clear to one skilled in the art, that the pulse trains in these two examples take into account both the threshold properties and the refractory properties of different types of nerve fibers which were shown in FIG. 5.

The pulses and pulse trains of this disclosure gives physicians a lot of flexibility for trying various different neuromodulation algorithms, for providing and optimizing therapy for urological disorders (and fecal incontinence) disorders. Furthermore, as shown in conjunction with FIG. 36-I, a combination of tripolar electrodes with different pulse shapes may be used for selective stimulation of sacral nerve(s). The different pulses used in conjunction with tripolar electrodes are shown in conjunction with FIGS. 36J, 36K, 37L, 36M, 36N, and 36-O. This combination is advantageous, because it can be used to provide selective large fiber block as well.

The combination of tripolar electrodes and the pulse shapes of FIGS. 36-J to 36-O would not only decrease or prevent the unwanted side effects, but the electrical charge of the pulse is also reduced, which will make this technique safer for long-term clinical applications.

In the tripolar cuff electrodes (FIG. 36-I), the electrode consists of a cathode, flanked by two anodes. When stimulation is applied, the nerve membrane is depolarized near the cathode and hyperpolarized near the anodes. If the membrane is sufficiently hyperpolarized, an action potential (AP) that travels into the depolarized zone cannot pass the hyperpolarized zone and is arrested. As with excitation, a lower external stimulus is needed for blocking large diameter fibers than for blocking smaller ones (C-fibers). Therefore, by applying a current above the blocking threshold for the large fibers but below the blocking threshold for the smaller ones, selective activation of the small fibers can be obtained. This is one of the aims of this invention, where selective stimulation of C-fibers can be achieved, without the unwanted side effects of motor stimulation to the throat region.

As shown in FIGS. 36J and 36K, the microcontroller 398 in the pulse generator 391 is configured to provide stepped pulses. The current of the first step is too low to induce an action potential (AP), but only depolarizes the membrane. The AP is generated during the second step. The pulses in FIGS. 36J and 36K are similar, except that the pulses in FIG. 36J have a longer first step. In addition to anodel blocking, another advantage of these stepped pulses is that the total charge per pulse can be reduced by almost a third.

Other examples of complex pulses, that may be used with tripolar electrodes are shown in FIGS. 36-L to 36-O. FIG. 36L shows biphasic pulses with a time delay td between the positive and negative pulse. FIG. 36M shows biphasic pulses with a time delay td, where the second part of the pulse is a step pulse. FIG. 36N shows ramp pulses, and FIG. 36-O show pulses with exponential components. Theoretical work, computer modeling, and animal studies have all shown that lower charge is obtained with these modified pulses when compared to square pulses. The charge reduction of these pulses can be approximately 30% less when compared to square pulses, which is fairly significant. The microcontroller 398 of the pulse generator 391 can be configured to deliver these pulses, as is well known to one skilled in the art.

Since the number of types of pulses and pulse trains to provide therapy can be complex for many physician's, in one aspect pre-determined/pre-packaged program comprise a complete program for the pulse trains that deliver therapy. The advantage of the pre-packaged programs is that the physician may program a complicated program simply by selecting a program number.

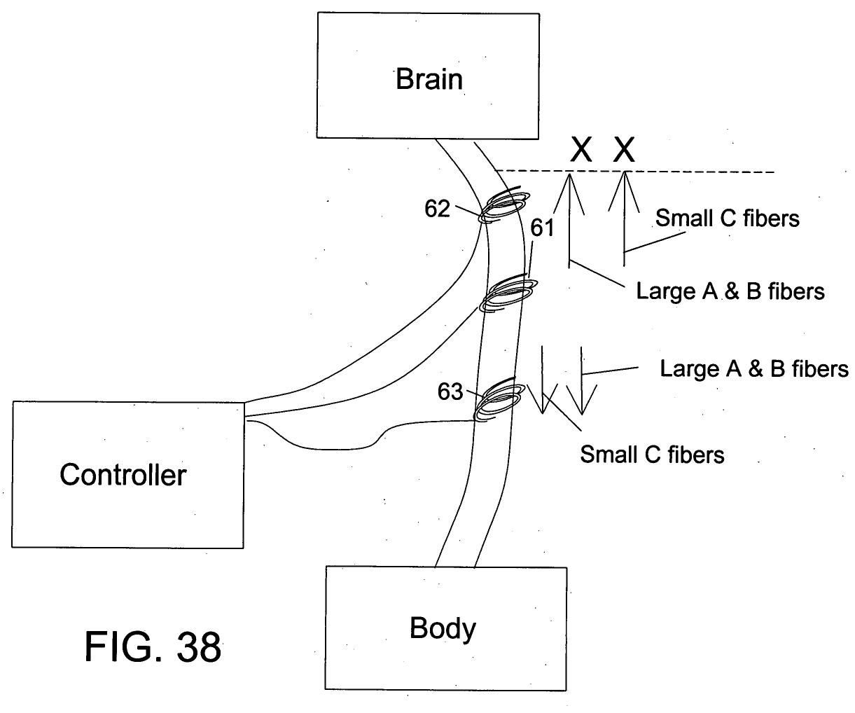

Since one of the objects of this invention is to decease side effects, blocking electrodes may be strategically placed at the relevant branches of sacral nerve(s). In one aspect efferent stimulation of selected types of fibers may be substantially blocked, utilizing the “greenwave” effect. In such a case, as shown in conjunction with FIG. 37, a tripolar lead is utilized. As depicted on the top portion of FIG. 37, a depolarization peak 10 on the sacral nerve(s) bundle corresponding to electrode 61 (cathode) and the two hyper-polarization peaks 8, 12 corresponding to electrodes 62, 63 (anodes). With the microcontroller controlling the tripolar device, the size and timing of the hyper-polarizations 8, 12 can be controlled. As was shown previously in FIG. 5, since the speed of conduction is different between the larger diameter A and B fibers and the smaller diameter c-fibers, by appropriately timing the pulses, collision blocks can be created for conduction via the large diameter A and B fibers in the efferent direction. This is depicted schematically in FIG. 38. Alternatively, separate leads may be utilized for stimulation and blocking, and the pulse generator may be adapted for two or three leads, as is well known in the art for dual chamber cardiac pacemakers or implantable defibrillators.

Therefore in the method and system of this invention, stimulation without block may be provided. Additionally, stimulation with selective block may be provided. Blocking of nerve impulses, unidirectional blocking, and selective blocking of nerve impulses is well known in the scientific literature. Some of the general literature is listed below and is incorporated herein by reference. (a) “Generation of unidirectionally propagating action potentials using a monopolar electrode cuff”, Annals of Biomedical Engineering, volume 14, pp. 437-450, By Ira J. Ungar et al. (b) “An asymmetric two electrode cuff for generation of unidirectionally propagated action potentials”, IEEE Transactions on Biomedical Engineering, volume BME-33, No. 6, June 1986, By James D. Sweeney, et al. (c) A spiral nerve cuff electrode for peripheral nerve stimulation, IEEE Transactions on Biomedical Engineering, volume 35, No. 11, November 1988, By Gregory G. Naples. et al. (d) “A nerve cuff technique for selective excitation of peripheral nerve trunk regions, IEEE Transactions on Biomedical Engineering, volume 37, No. 7, July 1990, By James D. Sweeney, et al. (e) “Generation of unidirectionally propagated action potentials in a peripheral nerve by brief stimuli”, Science, volume 206 pp. 1311-1312, Dec. 14, 1979, By Van Den Honert et al. (f) “A technique for collision block of perpheral nerve: Frequency dependence” IEEE Transactions on Biomedical Engineering, MP-12, volume 28, pp. 379-382, 1981, By Van Den Honert et al. (g) “A nerve cuff design for the selective activation and blocking of myelinated nerve fibers” Ann. Conf. of the IEEE Engineering in Medicine and Biology Soc., volume 13, No. 2, p 906, 1991, By D. M Fitzpatrick et al. (h) “Orderly recruitment of motoneurons in an acute rabbit model”, “Ann. Conf. of the IEEE Engineering in Medicine and Biology Soc., volume 20, No. 5, page 2564, 1998, By N. J. M. Rijkhof, et al. (i) “Orderly stimulation of skeletal muscle motor units with tripolar nerve cuff electrode”, IEEE Transactions on Biomedical Engineering, volume 36, No. 8, pp. 836, 1989, By R. Bratta. (j) M. Devor, “Pain Networks”, Handbook of Brand Theory and Neural Networks, Ed. M. A. Arbib, MIT Press, page 698, 1998.

Blocking can be generally divided into 3 categories: (a) DC or anodal block, (b) Wedenski Block, and (c) Collision block. In anodal block there is a steady potential which is applied to the nerve causing a reversible and selective block. In Wedenski Block the nerve is stimulated at a high rate causing the rapid depletion of the neurotransmitter. In collision blocking, unidirectional action potentials are generated anti-dromically. The maximal frequency for complete block is the reciprocal of the refractory period plus the transit time, i.e. typically less than a few hundred hertz. The use of any of these blocking techniques can be applied for the practice of this invention, and all are considered within the scope of this invention.

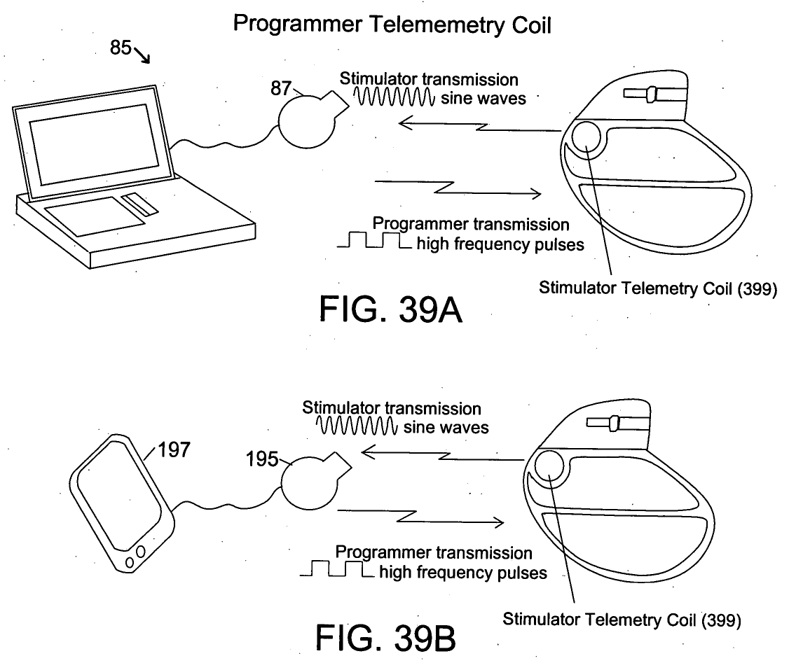

The programming of the implanted pulse generator (IPG) 391 is shown in conjunction with FIGS. 39A and 39B. With the magnetic Reed Switch 389 (FIG. 28A) in the closed position, a coil in the head of the programmer 85, communicates with a telemetry coil 399 of the implanted pulse generator 391. Bi-directional inductive telemetry is used to exchange data with the implanted unit 391 by means of the external programming unit 85.

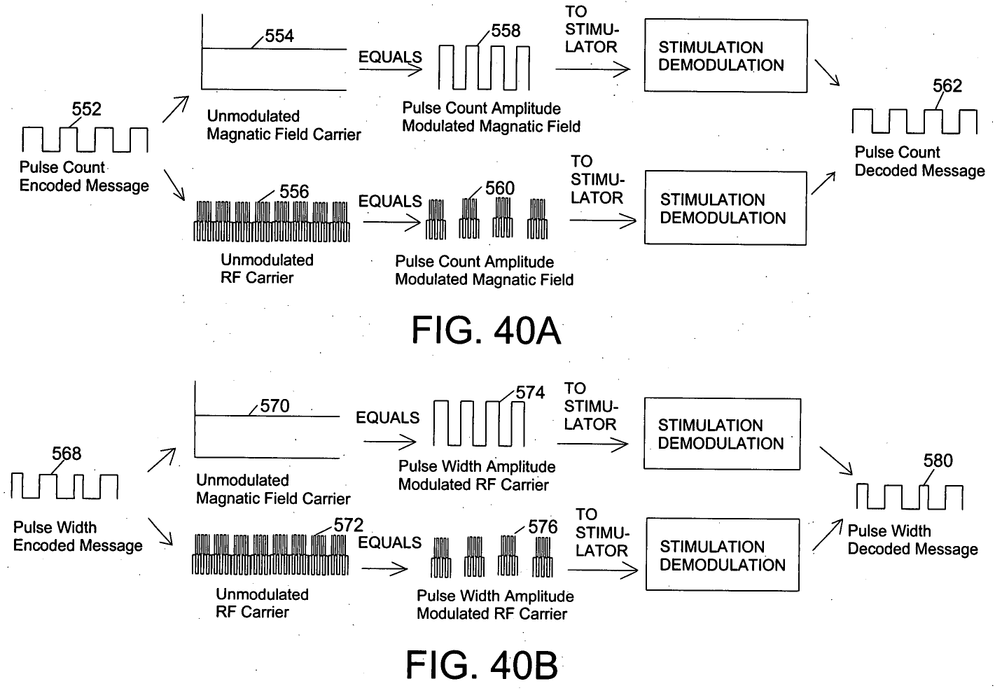

The transmission of programming information involves manipulation of the carrier signal in a manner that is recognizable by the pulse generator 391 as a valid set of instructions. The process of modulation serves as a means of encoding the programming instruction in a language that is interpretable by the implanted pulse generator 391. Modulation of signal amplitude, pulse width, and time between pulses are all used in the programming system, as will be appreciated by those skilled in the art. FIG. 40A shows an example of pulse count modulation, and FIG. 40B shows an example of pulse width modulation, that can be used for encoding.

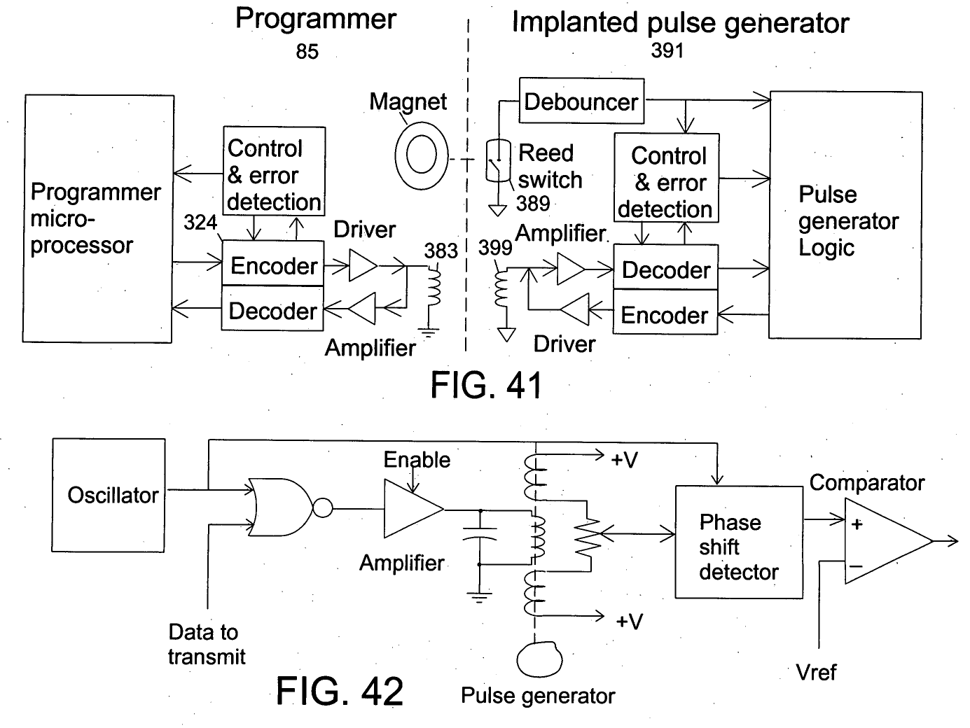

FIG. 41 shows a simplified overall block diagram of the implanted pulse generator (IPG) 391 programming and telemetry interface. The left half of FIG. 41 is programmer 85 which communicates programming and telemetry information with the IPG 391. The sections of the IPG 391 associated with programming and telemetry are shown on the right half of FIG. 41. In this case, the programming sequence is initiated by bringing a permanent magnet in the proximity of the IPG 391 which closes a reed switch 389 in the IPG 391. Information is then encoded into a special error-correcting pulse sequence and transmitted electromagnetically through a set of coils. The received message is decoded, checked for errors, and passed on to the unit's logic circuitry. The IPG 391 of this embodiment includes the capability of bi-directional communication.

The reed switch 389 is a magnetically-sensitive mechanical switch, which consists of two thin strips of metal (the “reed”) which are ferromagnetic. The reeds normally spring apart when no magnetic field is present. When a field is applied, the reeds come together to form a closed circuit because doing so creates a path of least reluctance. The programming head of the programmer contains a high-field-strength ceramic magnet.

When the switch closes, it activates the programming hardware, and initiates an interrupt of the IPG central processor. Closing the reed switch 389 also presents the logic used to encode and decode programming and telemetry signals. A nonmaskable interrupt (NMI) is sent to the IPG processor, which then executes special programming software. Since the NMI is an edge-triggered signal and the reed switch is vulnerable to mechanical bounce, a debouncing circuit is used to avoid multiple interrupts. The overall current consumption of the IPG increases during programming because of the debouncing circuit and other communication circuits.

A coil 399 is used as an antenna for both reception and transmission. Another set of coils 383 is placed in the programming head, a relatively small sized unit connected to the programmer 85. All coils are tuned to the same resonant frequency. The interface is half-duplex with one unit transmitting at a time.

Since the relative positions of the programming head 87 and IPG 391 determine the coupling of the coils, this embodiment utilizes a special circuit which has been devised to aid the positioning of the programming head, and is shown in FIG. 42. It operates on similar principles to the linear variable differential transformer. An oscillator tuned to the resonant frequency of the pacemaker coil 399 drives the center coil of a three-coil set in the programmer head. The phase difference between the original oscillator signal and the resulting signal from the two outer coils is measured using a phase shift detector. It is proportional to the distance between the implanted pulse generator and the programmer head. The phase shift, as a voltage, is compared to a reference voltage and is then used to control an indicator such as an LED. An enable signal allows switching the circuit on and off.

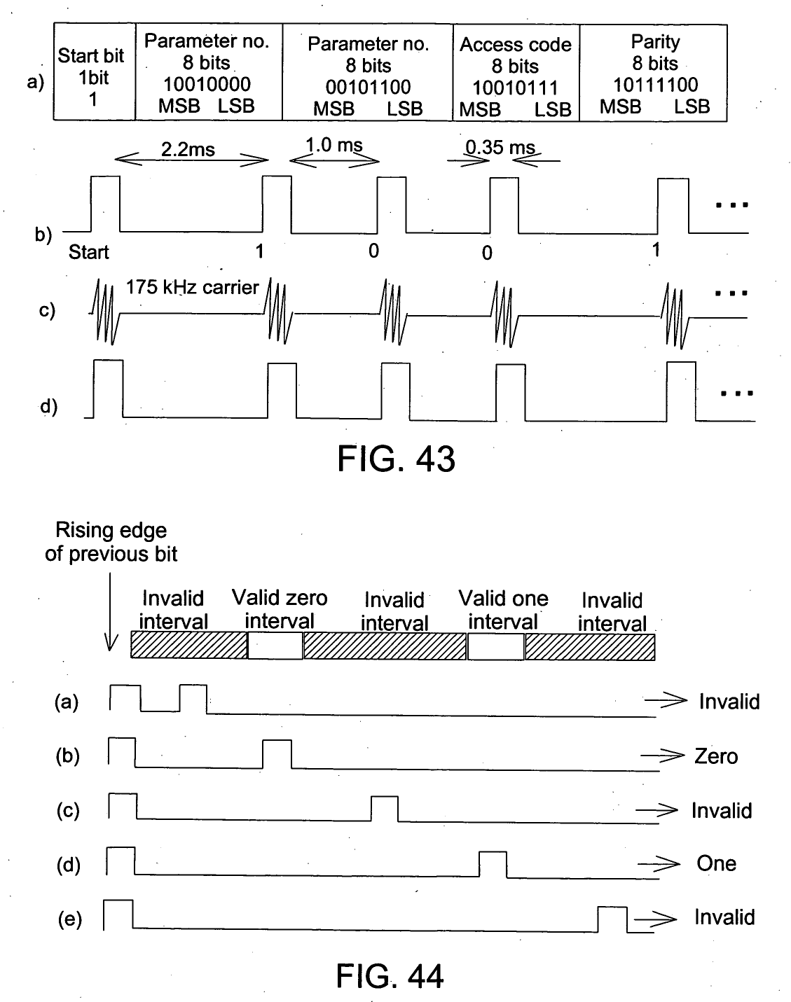

Actual programming is shown in conjunction with FIGS. 43 and 44. Programming and telemetry messages comprise many bits; however, the coil interface can only transmit one bit at a time. In addition, the signal is modulated to the resonant frequency of the coils, and must be transmitted in a relatively short period of time, and must provide detection of erroneous data.

A programming message is comprised of five parts FIG. 43(a). The start bit indicates the beginning of the message and is used to synchronize the timing of the rest of the message. The parameter number specifies which parameter (e.g., mode, pulse width, delay) is to be programmed. In the example, in FIG. 43(a) the number 10010000 specifies the pulse rate to be specified. The parameter value represents the value that the parameter should be set to. This value may be an index into a table of possible values; for example, the value 00101100 represents a pulse stimulus rate of 80 pulses/min. The access code is a fixed number based on the stimulus generator model which must be matched exactly for the message to succeed. It acts as a security mechanism against use of the wrong programmer, errors in the message, or spurious programming from environmental noise. It can also potentially allow more than one programmable implant in the patient. Finally, the parity field is the bitwise exclusive-OR of the parameter number and value fields. It is one of several error-detection mechanisms.

All of the bits are then encoded as a sequence of pulses of 0.35-ms duration FIG. 43(b). The start bit is a single pulse. The remaining bits are delayed from their previous bit according to their bit value. If the bit is a zero, the delay is short (1.0); if it is a one, the delay is long (2.2 ms). This technique of pulse position coding, makes detection of errors easier.

The serial pulse sequence is then amplitude modulated for transmission FIG. 43(c). The carrier frequency is the resonant frequency of the coils. This signal is transmitted from one set of coils to the other and then demodulated back into a pulse sequence FIG. 43(d).

FIG. 44 shows how each bit of the pulse sequence is decoded from the demodulated signal. As soon as each bit is received, a timer begins timing the delay to the next pulse. If the pulse occurs within a specific early interval, it is counted as a zero bit (FIG. 44(b)). If it otherwise occurs with a later interval, it is considered to be a one bit (FIG. 44(d)). Pulses that come too early, too late, or between the two intervals are considered to be errors and the entire message is discarded (FIG. 44 (a, c, e)). Each bit begins the timing of the bit that follows it. The start bit is used only to time the first bit.

Telemetry data may be either analog or digital. Digital signals are first converted into a serial bit stream using an encoding such as shown in FIG. 44 (b). The serial stream or the analog data is then frequency modulated for transmission.

An advantage of this and other encodings is that they provide multiple forms of error detection. The coils and receiver circuitry are tuned to the modulation frequency, eliminating noise at other frequencies. Pulse-position coding can detect errors by accepting pulses only within narrowly-intervals. The access code acts as a security key to prevent programming by spurious noise or other equipment. Finally, the parity field and other checksums provides a final verification that the message is valid. At any time, if an error is detected, the entire message is discarded.

Another more sophisticated type of pulse position modulation may be used to increase the bit transmission rate. In this, the position of a pulse within a frame is encoded into one of a finite number of values, e.g. 16. A special synchronizing bit is transmitted to signal the start of the frame. Typically, the frame contains a code which specifies the type or data contained in the remainder of the frame.

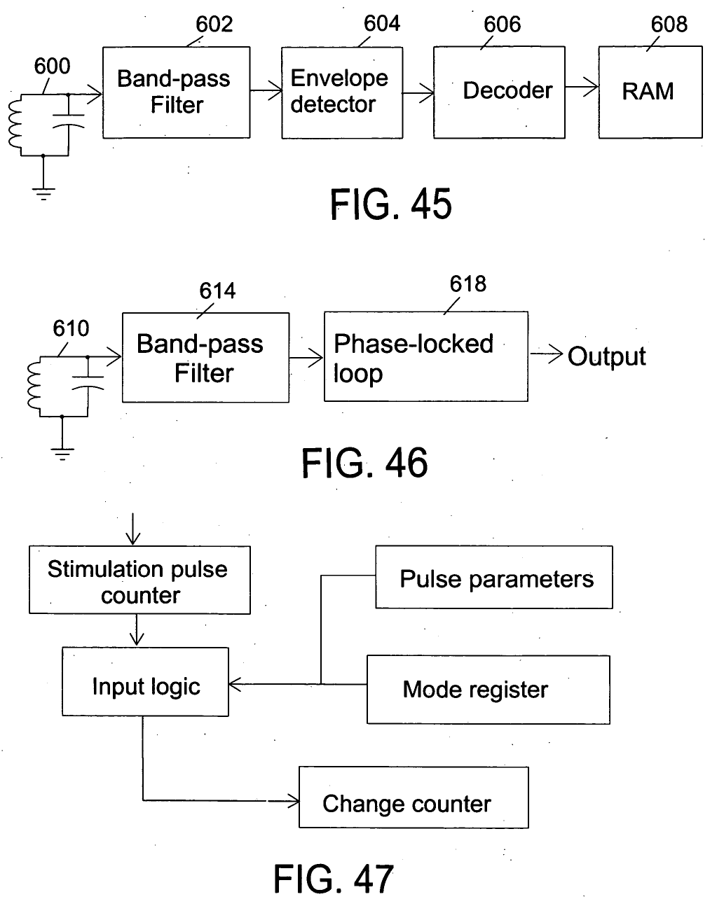

FIG. 45 shows a diagram of receiving and decoding circuitry for programming data. The IPG coil, in parallel with capacitor creates a tuned circuit for receiving data. The signal is band-pass filtered 602 and envelope detected 604 to create the pulsed signal in FIG. 43 (d). After decoding, the parameter value is placed in a RAM at the location specified by the parameter number. The IPG can have two copies of the RAM—a permanent set and a temporary set—which makes it easy for the physician to set the IPG to a temporary configuration and later reprogram it back to the usual settings.

FIG. 46 shows the basic circuit used to receive telemetry data. Again, a coil and capacitor create a resonant circuit tuned to the carrier frequency. The signal is further band-pass filtered 614 and then frequency-demodulated using a phase-locked loop 618.

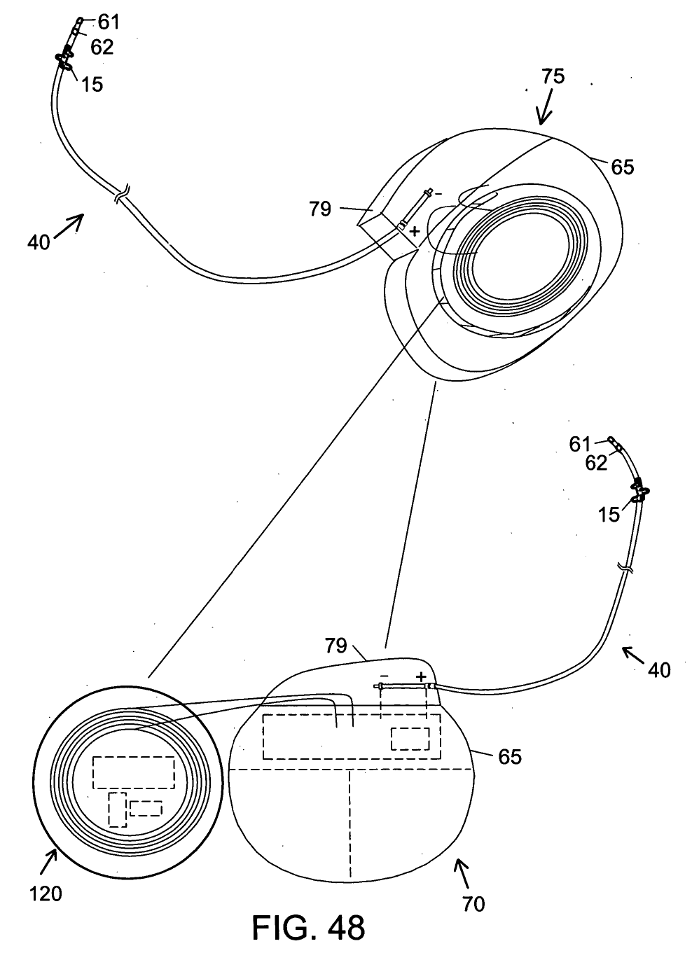

This embodiment also comprises an optional battery status test circuit. Shown in conjunction with FIG. 47, the charge delivered by the battery is estimated by keeping track of the number of pulses delivered by the IPG 391. An internal charge counter is updated during each test mode to read the total charge delivered. This information about battery status is read from the IPG 391 via telemetry.

### Combination Implantable Device Comprising Both a Stimulus-Receiver and a Programmable Implantable Pulse Generator (IPG)

In one embodiment, the implantable device may comprise both a stimulus-receiver and a programmable implantable pulse generator (IPG) in one device. Another embodiment of a similar device is disclosed in applicant's co-pending application Ser. No. 10/436,006. This embodiment also comprises predetermined/pre-packaged programs. Examples of several stimulation states were given in the previous sections, under “Programmer-less Implantable Pulse Generator (IPG)” and “Programmable Implantable Pulse Generator”. These predetermined/pre-packaged programs comprise unique combinations of pulse amplitude, pulse width, pulse frequency, ON-time and OFF-time.

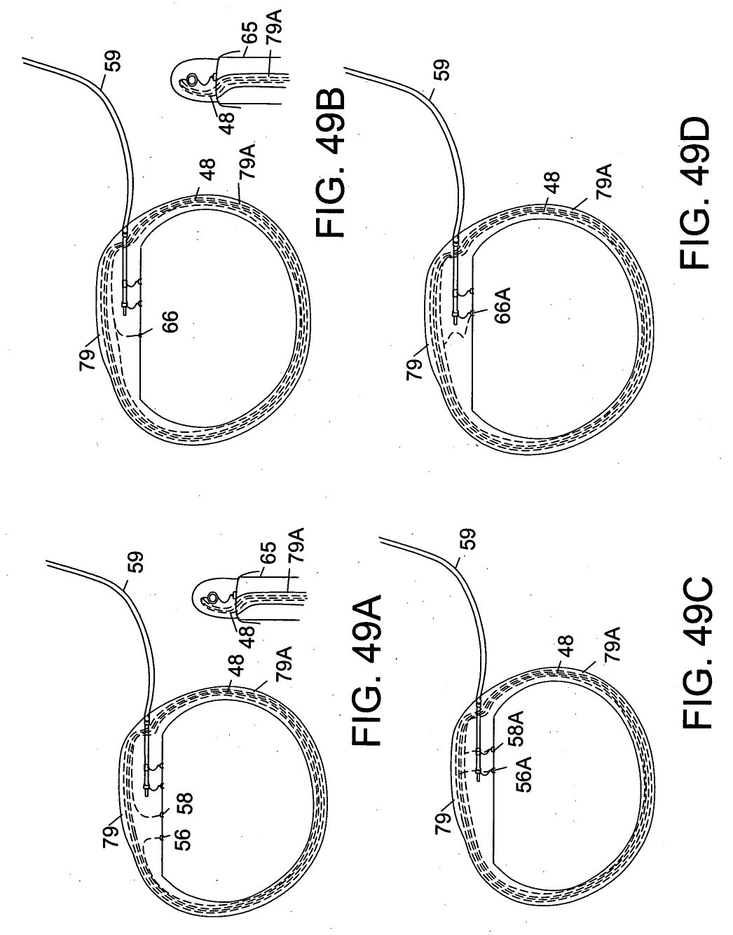

FIG. 48 shows a close up view of the packaging of the implanted stimulator 75 of this embodiment, showing the two subassemblies 120, 170. The two subassemblies are the stimulus-receiver module 120 and the battery operated pulse generator module 170. The electrical components of the stimulus-receiver module 120 may be substantially in the titanium case along with other circuitry, except for a coil. The coil may be outside the titanium case as shown in FIG. 48, or the coil 48C may be externalized at the header portion 79 of the implanted device, and may be wrapped around the titanium can. In this case, the coil is encased in the same material as the header 79, as shown in FIGS. 49A-49D. FIG. 49A depicts a bipolar configuration with two separate feed-throughs, 56, 58. FIG. 49B depicts a unipolar configuration with one separate feed-through 66. FIGS. 49C, and 49D depict the same configuration except the feed-throughs are common with the feed-throughs 66A for the lead.

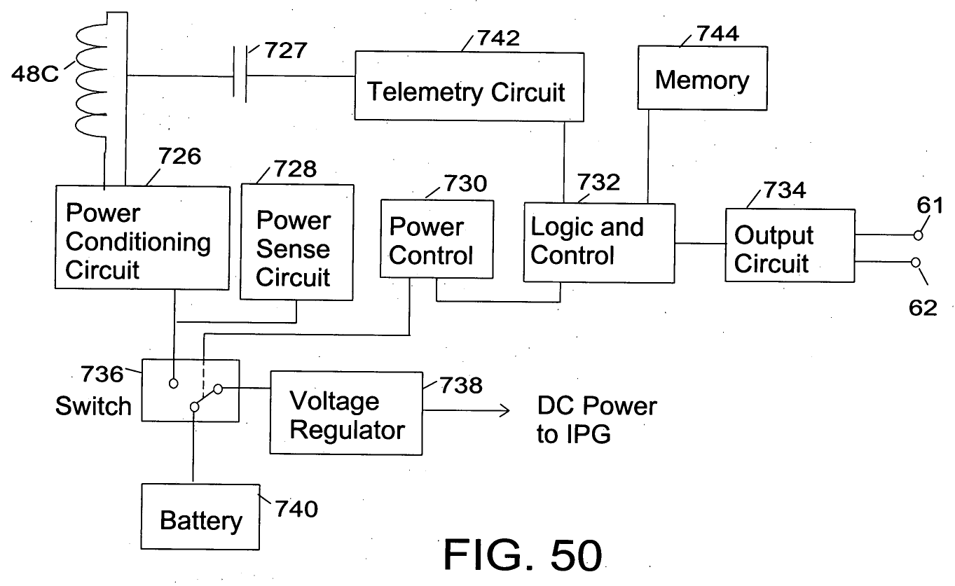

FIG. 50 is a simplified overall block diagram of the embodiment where the implanted stimulator 75 is a combination device, which may be used as a stimulus-receiver (SR) in conjunction with an external stimulator, or the same implanted device may be used as a traditional programmable implanted pulse generator (IPG). The coil 48C which is external to the titanium case may be used both as a secondary of a stimulus-receiver, or may also be used as the forward and back telemetry coil.

In this embodiment, as disclosed in FIG. 50, the IPG circuitry within the titanium case is used for all stimulation pulses whether the energy source is the internal battery 740 or an external power source. The external device serves as a source of energy, and as a programmer that sends telemetry to the IPG. For programming, the energy is sent as high frequency sine waves with superimposed telemetry wave driving the external coil 46C. Once received by the implanted coil 48C, the telemetry is passed through coupling capacitor 727 to the IPG's telemetry circuit 742. For pulse delivery using external power source, the stimulus-receiver portion will receive the energy coupled to the implanted coil 48C and, using the power conditioning circuit 726, rectify it to produce DC, filter and regulate the DC, and couple it to the IPG's voltage regulator 738 section so that the IPG can run from the externally supplied energy rather than the implanted battery 740.

The system provides a power sense circuit 728 that senses the presence of external power communicated with the power control 730 when adequate and stable power is available from an external source. The power control circuit controls a switch 736 that selects either battery power 740 or conditioned external power from 726. The logic and control section 732 and memory 744 includes the IPG's microcontroller, pre-programmed instructions, and stored chagneable parameters. Using input for the telemetry circuit 742 and power control 730, this section controls the output circuit 734 that generates the output pulses.

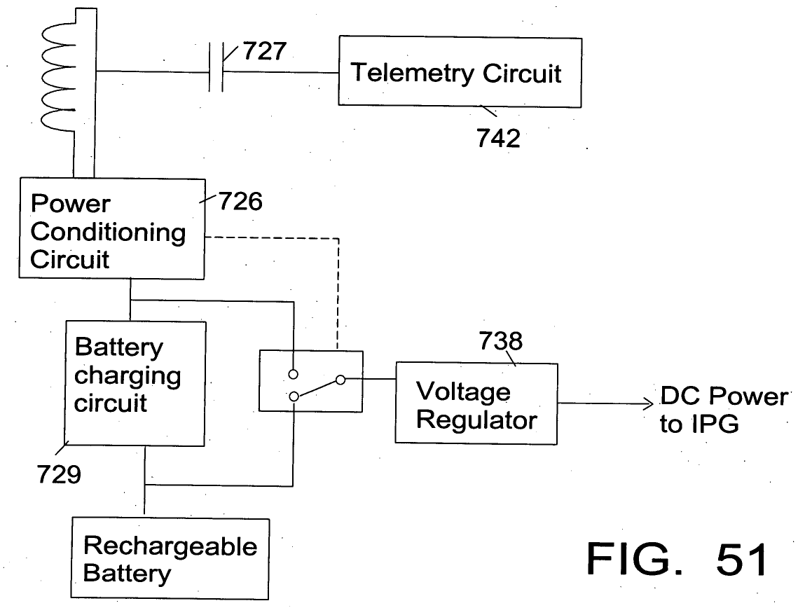

It will be clear to one skilled in the art that this embodiment of the invention can also be practiced with a rechargeable battery. This version is shown in conjunction with FIG. 51. The circuitry in the two versions are similar except for the battery charging circuitry 749. This circuit is energized when external power is available. It senses the charge state of the battery and provides appropriate charge current to safely recharge the battery without overcharging.

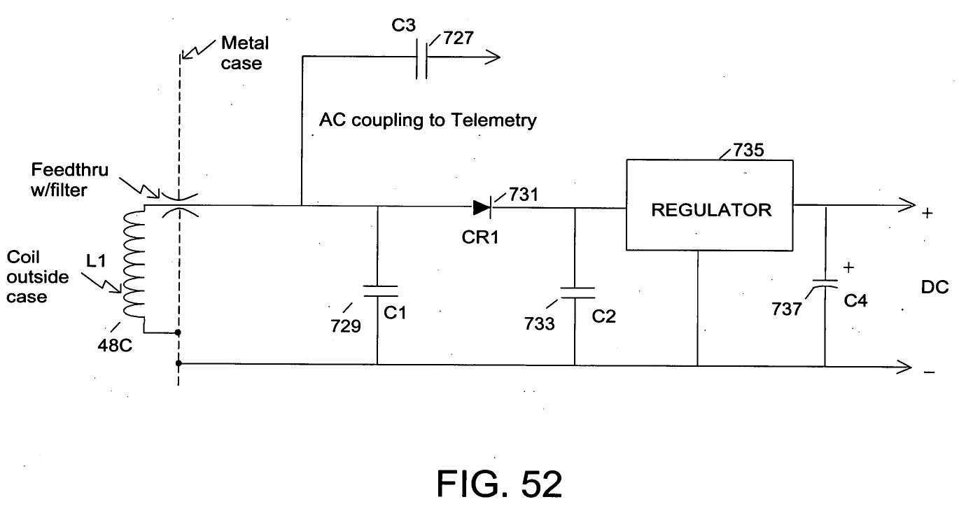

The stimulus-receiver portion of the circuitry is shown in conjunction with FIG. 52. Capacitor C1 (729) makes the combination of C1 and L1 sensitive to the resonant frequency and less sensitive to other frequencies, and energy from an external (primary) coil 46C is inductively transferred to the implanted unit via the secondary coil 48C. The AC signal is rectified to DC via diode 731, and filtered via capacitor 733. A regulator 735 sets the output voltage and limits it to a value just above the maximum IPG cell voltage. The output capacitor C4 (737), typically a tantalum capacitor with a value of 100 micro-Farads or greater, stores charge so that the circuit can supply the IPG with high values of current for a short time duration with minimal voltage change during a pulse while the current draw from the external source remains relatively constant. Also shown in conjunction with FIG. 52, a capacitor C3 (727) couples signals for forward and back telemetry.

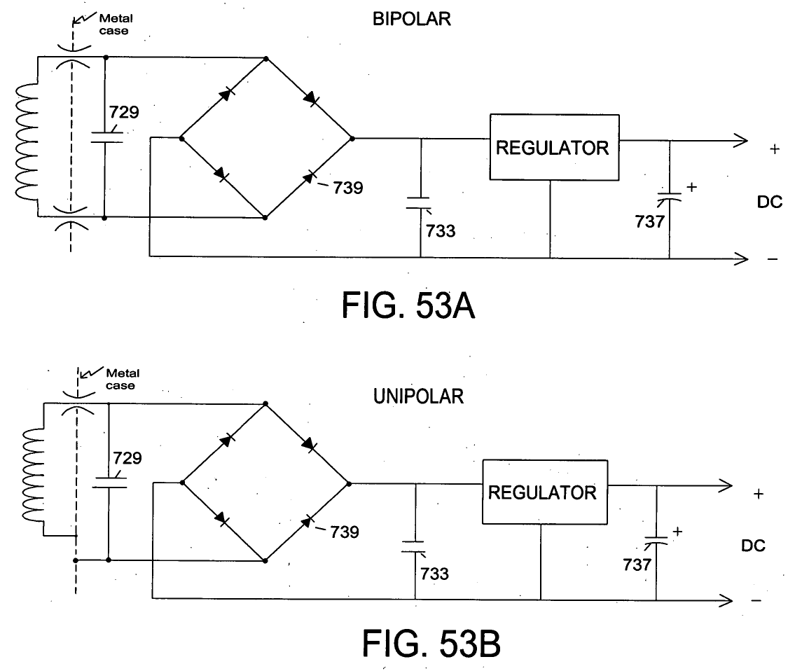

FIGS. 53A and 53B show alternate connection of the receiving coil. In FIG. 53A, each end of the coil is connected to the circuit through a hermetic feedthrough filter. In this instance, the DC output is floating with respect to the IPG's case. In FIG. 53B, one end of the coil is connected to the exterior of the IPG's case. The circuit is completed by connecting the capacitor 729 and bridge rectifier 739 to the interior of the IPG's case The advantage of this arrangement is that it requires one less hermetic feedthrough filter, thus reducing the cost and improving the reliability of the IPG. Hermetic feedthrough filters are expensive and a possible failure point. However, the case connection may complicit the output circuitry or limit its versatility. When using a bipolar electrode, care must be taken to prevent an unwanted return path for the pulse to the IPG's case. This is not a concern for unipolar pulses using a single conductor electrode because it relies on the IPG's case a return for the pulse current.

In the unipolar configuration, advantageously a bigger tissue area is stimulated since the difference between the tip (cathode) and case (anode) is larger. Stimulation using both configuration is considered within the scope of this invention.

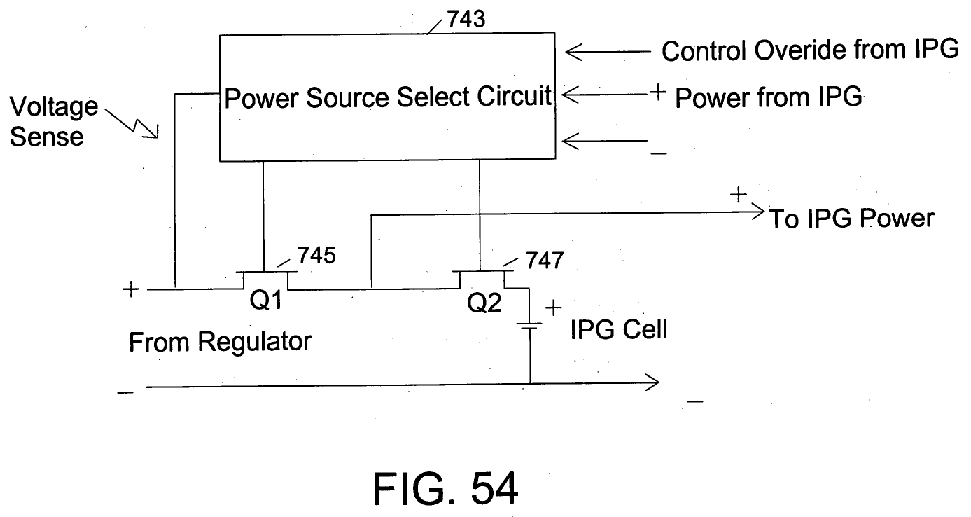

The power source select circuit is highlighted in conjunction with FIG. 54. In this embodiment, the IPG provides stimulation pulses according to the stimulation programs stored in the memory 744 of the implanted stimulator, with power being supplied by the implanted battery 740. When stimulation energy from an external stimulator is inductively received via secondary coil 48C, the power source select circuit (shown in block 743) switches power via transistor Q1 745 and transistor Q2 743. Transistor Q1 and Q2 are preferably low loss MOS transistor used as switches, even though other types of transistors may be used.

### Implantable Pulse Generator (IPG) Comprising a Rechargable Battery

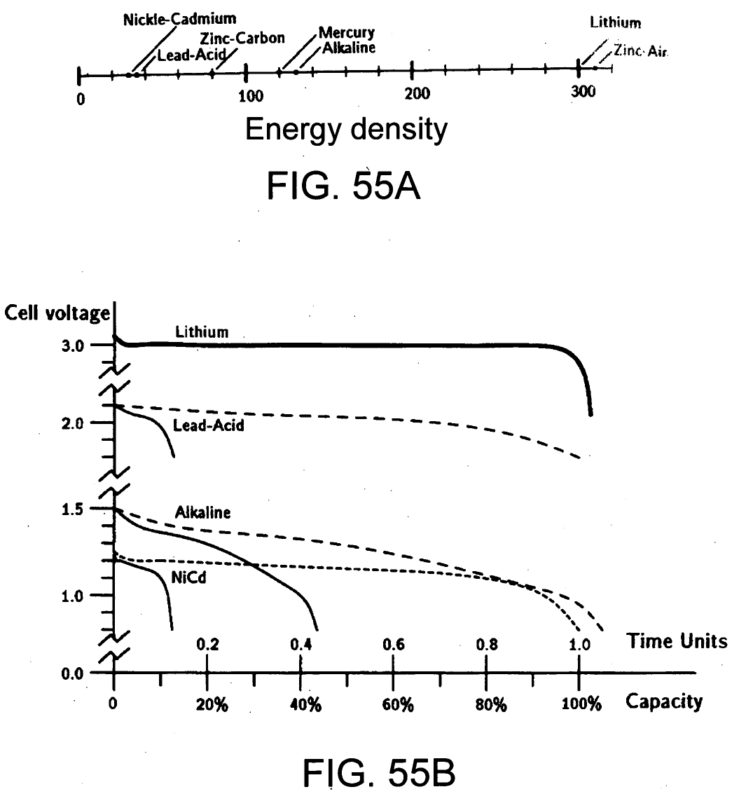

In one embodiment, an implantable pulse generator with rechargeable power source can be used. Because of the rapidity of the pulses required for modulating nerve tissue 54 with stimulating and/or blocking pulses, there is a real need for power sources that will provide an acceptable service life under conditions of continuous delivery of high frequency pulses. FIG. 55A shows a graph of the energy density of several commonly used battery technologies. Lithium batteries have by far the highest energy density of commonly available batteries. Also, a lithium battery maintains a nearly constant voltage during discharge. This is shown in conjunction with FIG. 55B, which is normalized to the performance of the lithium battery. Lithium-ion batteries also have a long cycle life, and no memory effect. However, Lithium-ion batteries are not as tolerant to overcharging and overdischarging. One of the most recent development in rechargable battery technology is the Lithium-ion polymer battery. Recently the major battery manufacturers (Sony, Panasonic, Sanyo) have announced plans for Lithium-ion polymer battery production.

This embodiment also comprises predetermined/pre-packaged programs. Examples of several stimulation states were given in the previous sections, under “Programmer-less Implantable Pulse Generator (IPG)” and “Programmable Implantable Pulse Generator”. These pre-packaged/pre-determined programs comprise unique combinations of pulse amplitude, pulse width, pulse frequency, ON-time and OFF-time. Additionally, predetermined programs comprising blocking pulses may also be stored in the memory of the pulse generator.

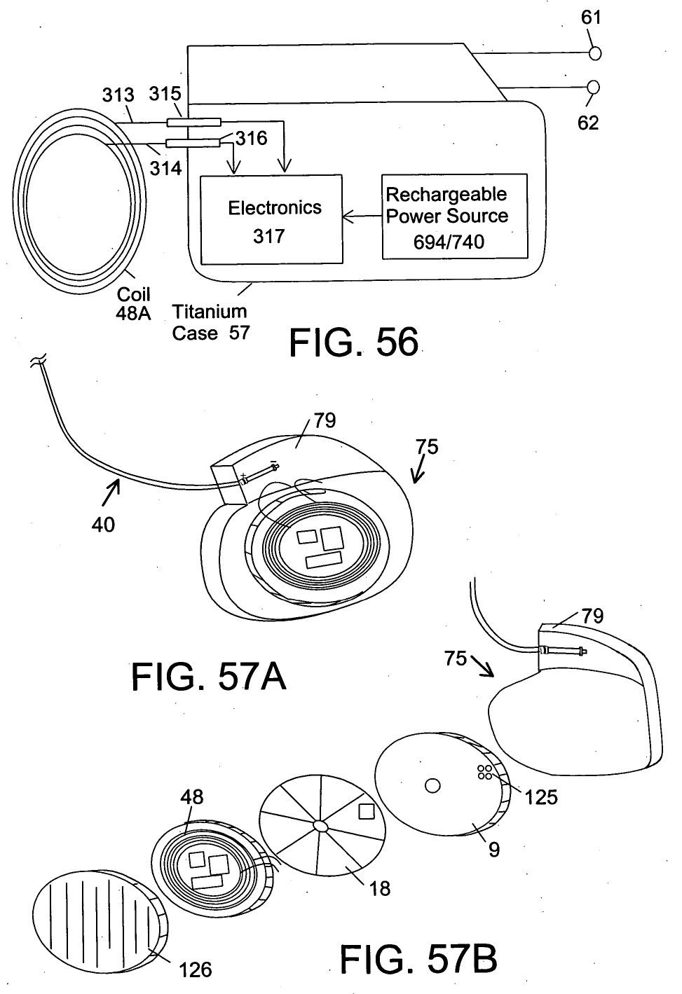

As shown in conjunction with FIG. 56, the coil is externalized from the titanium case 57. The RF pulses transmitted via coil 46 and received via subcutaneous coil 48A are rectified via a diode bridge. These DC pulses are processed and the resulting current applied to recharge the battery 694/740 in the implanted pulse generator. In one embodiment the coil 48C may be externalized at the header portion 79 of the implanted device, and may be wrapped around the titanium can, as was previously shown in FIGS. 49A-D.

In one embodiment, the coil may also be positioned on the titanium case as shown in conjunction with FIGS. 57A and 57B. FIG. 57A shows a diagram of the finished implantable stimulator 391R of one embodiment. FIG. 57B shows the pulse generator with some of the components used in assembly in an exploded view. These components include a coil cover 126, the secondary coil 48 and associated components, a magnetic shield 7, and a coil assembly carrier 9. The coil assembly carrier 9 has at least one positioning detail 125 located between the coil assembly and the feed through for positioning the electrical connection. The positioning detail 125 secures the electrical connection.

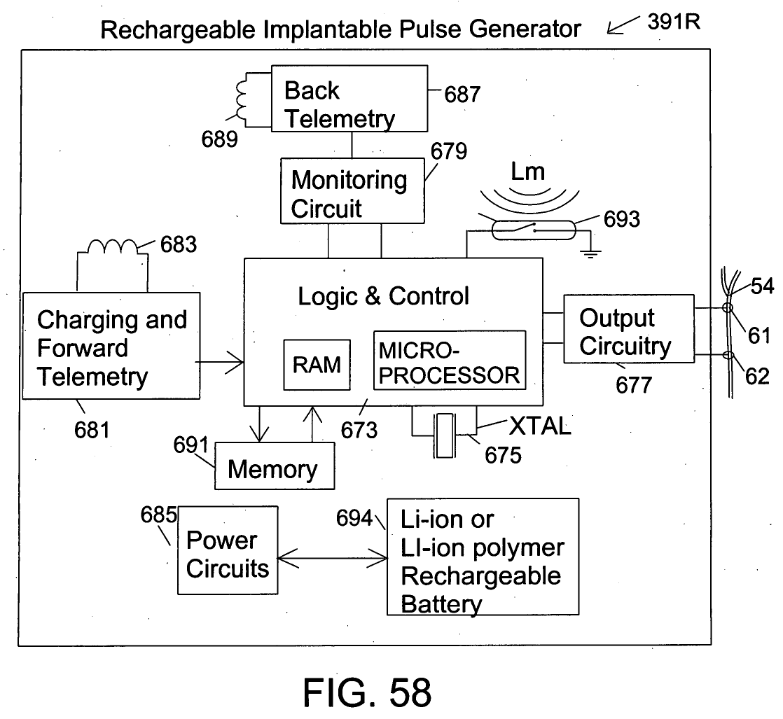

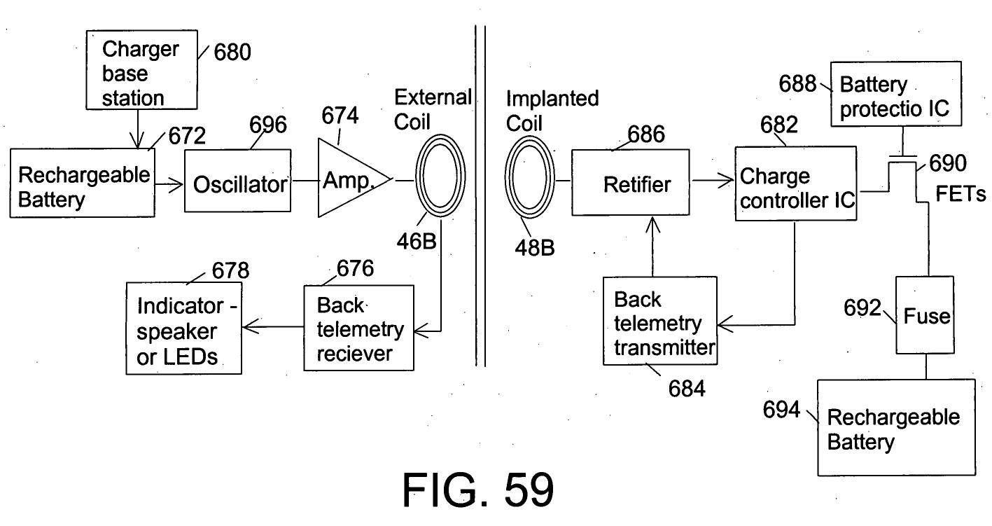

A schematic diagram of the implanted pulse generator (IPG 391R), with re-chargeable battery 694, is shown in conjunction with FIG. 58. The IPG 391R includes logic and control circuitry 673 connected to memory circuitry 691. The operating program and stimulation parameters are typically stored within the memory 691 via forward telemetry. Stimulation pulses are provided to the nerve tissue 54 via output circuitry 677 controlled by the microcontroller.

The operating power for the IPG 391R is derived from a rechargeable power source 694. The rechargeable power source 694 comprises a rechargeable lithium-ion or lithium-ion polymer battery. Recharging occurs inductively from an external charger to an implanted coil 48B underneath the skin 60. The rechargeable battery 694 may be recharged repeatedly as needed. Additionally, the IPG 391R is able to monitor and telemeter the status of its rechargable battery 691 each time a communication link is established with the external programmer 85.

Much of the circuitry included within the IPG 391R may be realized on a single application specific integrated circuit (ASIC). This allows the overall size of the IPG 391R to be quite small, and readily housed within a suitable hermetically-sealed case. The IPG case is preferably made from a titanium and is shaped in a rounded case.

Shown in conjunction with FIG. 59 are the recharging elements of this embodiment. The re-charging system uses a portable external charger to couple energy into the power source of the IPG 391R. The DC-to-AC conversion circuitry 696 of the re-charger receives energy from a battery 672 in the re-charger. A charger base station 680 and conventional AC power line may also be used. The AC signals amplified via power amplifier 674 are inductively coupled between an external coil 46B and an implanted coil 48B located subcutaneously with the implanted pulse generator (IPG) 391R. The AC signal received via implanted coil 48B is rectified 686 to a DC signal which is used for recharging the rechargeable battery 694 of the IPG, through a charge controller IC 682. Additional circuitry within the IPG 391R includes, battery protection IC 688 which controls a FET switch 690 to make sure that the rechargeable battery 694 is charged at the proper rate, and is not overcharged. The battery protection IC 688 can be an off-the-shelf IC available from Motorola (part no. MC 33349N-3R1). This IC monitors the voltage and current of the implanted rechargeable battery 694 to ensure safe operation. If the battery voltage rises above a safe maximum voltage, the battery protection IC 688 opens charge enabling FET switches 690, and prevents further charging. A fuse 692 acts as an additional safeguard, and disconnects the battery 694 if the battery charging current exceeds a safe level. As also shown in FIG. 59, charge completion detection is achieved by a back-telemetry transmitter 684, which modulates the secondary load by changing the full-wave rectifier into a half-wave rectifier/voltage clamp. This modulation is in turn, sensed by the charger as a change in the coil voltage due to the change in the reflected impedance. When detected through a back telemetry receiver 676, either an audible alarm is generated or a LED is turned on.

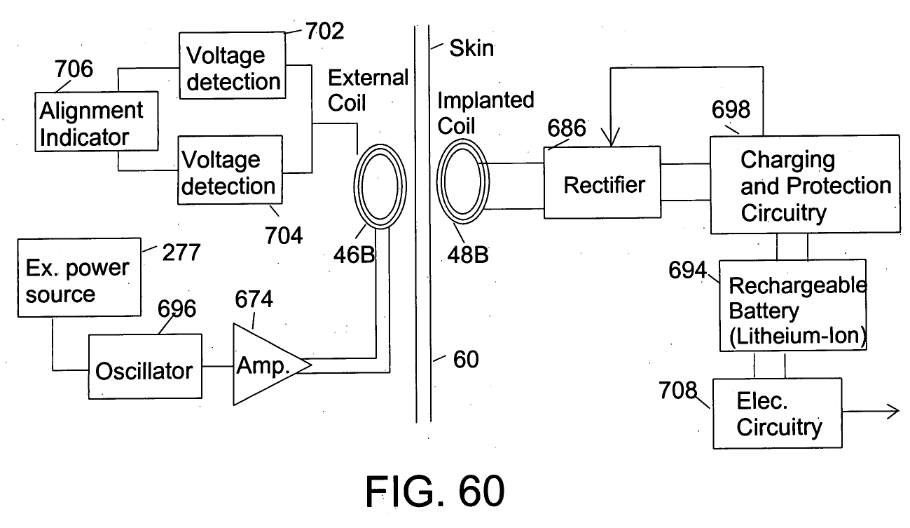

A simplified block diagram of charge completion and misalignment detection circuitry is shown in conjunction with FIG. 60. As shown, a switch regulator 686 operates as either a full-wave rectifier circuit or a half-wave rectifier circuit as controlled by a control signal (CS) generated by charging and protection circuitry 698. The energy induced in implanted coil 48B (from external coil 46B) passes through the switch rectifier 686 and charging and protection circuitry 698 to the implanted rechargeable battery 694. As the implanted battery 694 continues to be charged, the charging and protection circuitry 698 continuously monitors the charge current and battery voltage. When the charge current and battery voltage reach a predetermined level, the charging and protection circuitry 698 triggers a control signal. This control signal causes the switch rectifier 686 to switch to half-wave rectifier operation. When this change happens, the voltage sensed by voltage detector 702 causes the alignment indicator 706 to be activated. This indicator 706 may be an audible sound or a flashing LED type of indicator.

The indicator 706 may similarly be used as a misalignment indicator. In normal operation, when coils 46B (external) and 48B (implanted) are properly aligned, the voltage Vₛ sensed by voltage detector 704 is at a minimum level because maximum energy transfer is taking place. If and when the coils 46B and 48B become misaligned, then less than a maximum energy transfer occurs, and the voltage Vₛ sensed by detection circuit 704 increases significantly. If the voltage Vₛ reaches a predetermined level, alignment indicator 706 is activated via an audible speaker and/or LEDs for visual feedback. After adjustment, when an optimum energy transfer condition is established, causing Vₛ to decrease below the predetermined threshold level, the alignment indicator 706 is turned off.

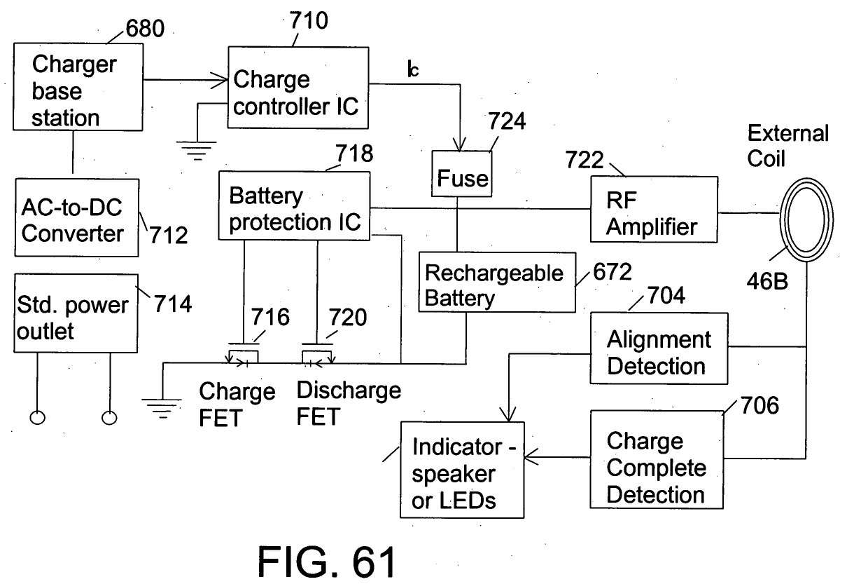

The elements of the external recharger are shown as a block diagram in conjunction with FIG. 61. In this disclosure, the words charger and recharger are used interchangeably. The charger base station 680 receives its energy from a standard power outlet 714, which is then converted to 5 volts DC by a AC-to-DC transformer 712. When the re-charger is placed in a charger base station 680, the re-chargeable battery 672 of the re-charger is fully recharged in a few hours and is able to recharge the battery 694 of the IPG 391R. If the battery 672 of the external re-charger falls below a prescribed limit of 2.5 volt DC, the battery 672 is trickle charged until the voltage is above the prescribed limit, and then at that point resumes a normal charging process.

As also shown in FIG. 81, a battery protection circuit 718 monitors the voltage condition, and disconnects the battery 672 through one of the FET switches 716, 720 if a fault occurs until a normal condition returns. A fuse 724 will disconnect the battery 672 should the charging or discharging current exceed a prescribed amount.

In summary, in the method of the current invention for neuromodulation of sacral nerve(s) 54, to provide adjunct therapy for urinary/fecal incontinence and urological disorders can be practiced with any of the several pulse generator systems disclosed including,

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a programmable implantable pulse generator;

e) a combination implantable device comprising both a stimulus-receiver and a programmable IPG; and

f) an IPG comprising a rechargeable battery.

Neuromodulation of sacral nerve(s) with any of these systems is considered within the scope of this invention.

### Remote Communications Module

In one embodiment, the external stimulator and/or the programmer has a telecommunications module, as described in a co-pending application, and summarized here for reader convenience. The telecommunications module has two-way communications capabilities.

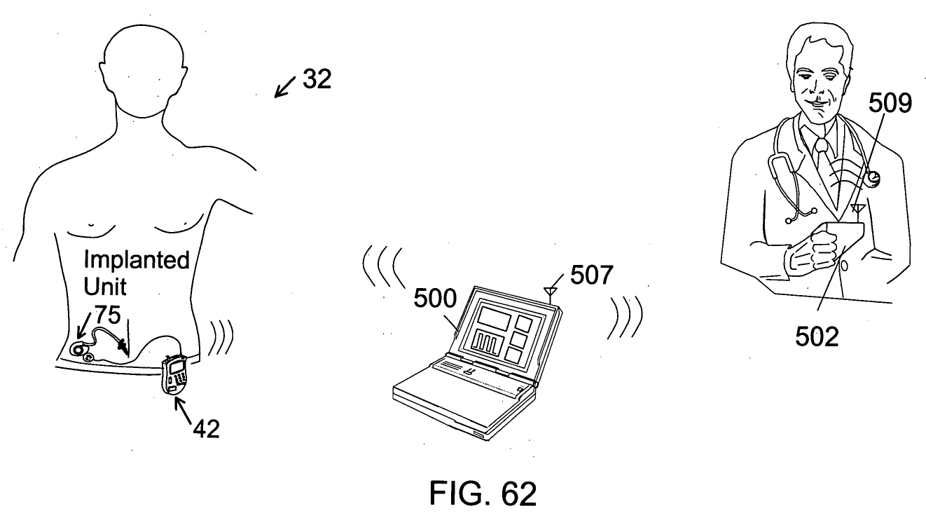

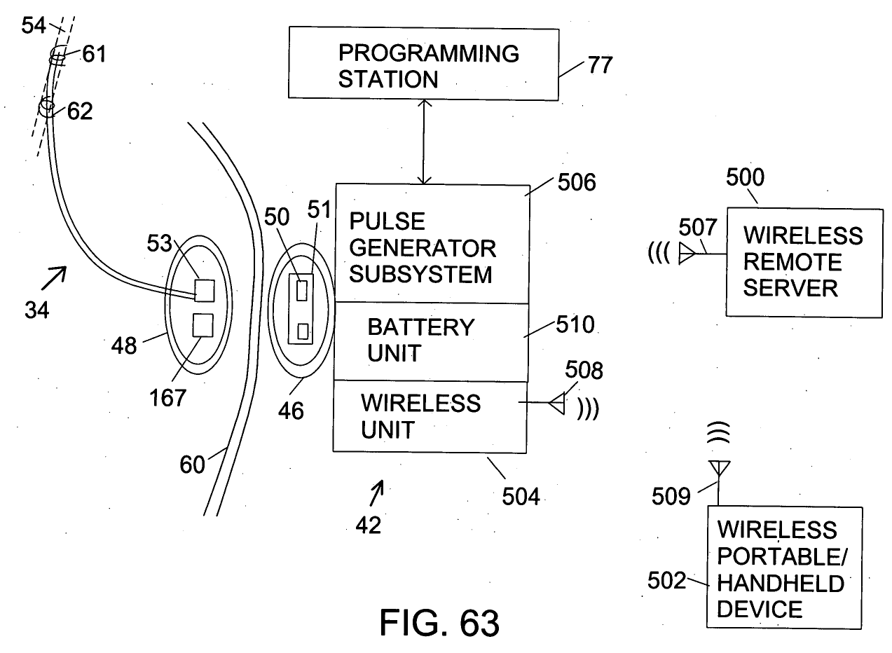

FIGS. 62 and 63 depict communication between an external stimulator 42 and a remote hand-held computer 502. A desktop or laptop computer can be a server 500 which is situated remotely, perhaps at a physician's office or a hospital. The stimulation parameter data can be viewed at this facility or reviewed remotely by medical personnel on a hand-held personal data assistant (PDA) 502, such as a “palm-pilot” from PALM corp. (Santa Clara, Calif.), a “Visor” from Handspring Corp. (Mountain view, Calif.) or on a personal computer (PC). The physician or appropriate medical personnel, is able to interrogate the external stimulator 42 device and know what the device is currently programmed to, as well as, get a graphical display of the pulse train. The wireless communication with the remote server 500 and hand-held PDA 502 would be supported in all geographical locations within and outside the United States (US) that provides cell phone voice and data communication service.

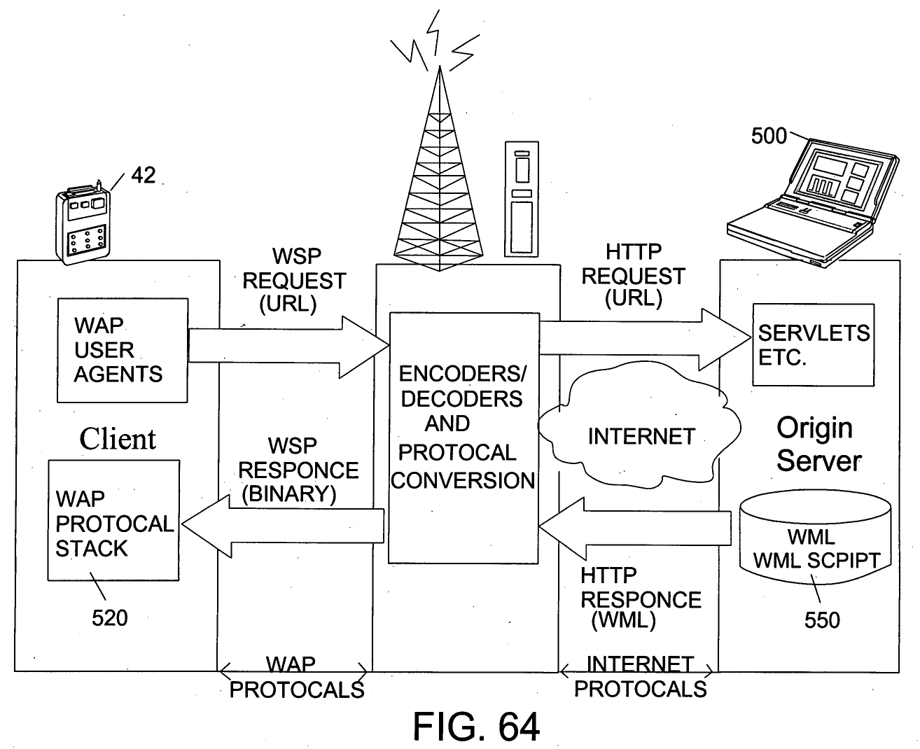

In one aspect of the invention, the telecommunications component can use Wireless Application Protocol (WAP). The Wireless Application Protocol (WAP), which is a set of communication protocols standardizing Internet access for wireless devices. While previously, manufacturers used different technologies to get Internet on hand-held devices, with WAP devices and services interoperate. WAP also promotes convergence of wireless data and the Internet. The WAP programming model is heavily based on the existing Internet programming model, and is shown schematically in FIG. 64. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops.

The key components of the WAP technology, as shown in FIG. 64, includes 1) Wireless Mark-up Language (WML) 550 which incorporates the concept of cards and decks, where a card is a single unit of interaction with the user. A service constitutes a number of cards collected in a deck. A card can be displayed on a small screen. WML supported Web pages reside on traditional Web servers. 2) WML Script which is a scripting language, enables application modules or applets to be dynamically transmitted to the client device and allows the user interaction with these applets. 3) Microbrowser, which is a lightweight application resident on the wireless terminal that controls the user interface and interprets the WML/WML Script content. 4) A lightweight protocol stack 520 which minimizes bandwidth requirements, guaranteeing that a broad range of wireless networks can run WAP applications. The protocol stack of WAP can comprise a set of protocols for the transport (WTP), session (WSP), and security (WTLS) layers. WSP is binary encoded and able to support header caching, thereby economizing on bandwidth requirements. WSP also compensates for high latency by allowing requests and responses to be handled asynchronously, sending before receiving the response to an earlier request. For lost data segments, perhaps due to fading or lack of coverage, WTP only retransmits lost segments using selective retransmission, thereby compensating for a less stable connection in wireless. The above mentioned features are industry standards adopted for wireless applications and greater details have been publicized, and well known to those skilled in the art.

In this embodiment, two modes of communication are possible. In the first, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device to download these parameters.

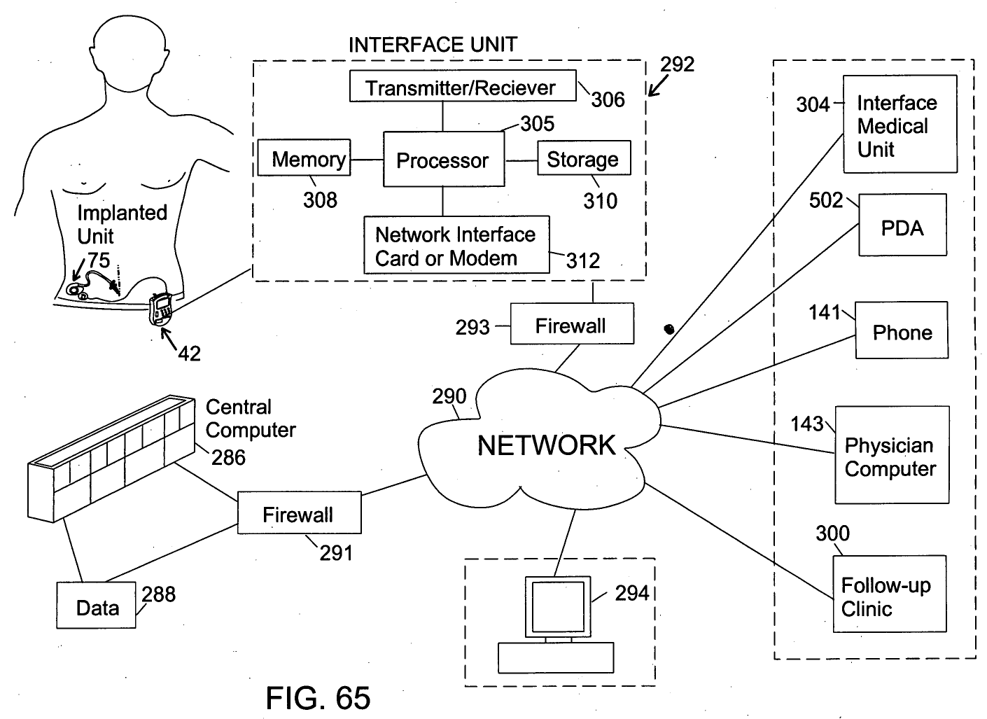

Shown in conjunction with FIG. 65, in one embodiment, the external stimulator 42 and/or the programmer 85 may also be networked to a central collaboration computer 286 as well as other devices such as a remote computer 294, PDA 502, phone 141, physician computer 143. The interface unit 292 in this embodiment communicates with the central collaborative network 290 via land-lines such as cable modem or wirelessly via the internet. A central computer 286 which has sufficient computing power and storage capability to collect and process large amounts of data, contains information regarding device history and serial number, and is in communication with the network 290. Communication over collaboration network 290 may be effected by way of a TCP/IP connection, particularly one using the internet, as well as a PSTN, DSL, cable modem, LAN, WAN or a direct dial-up connection.

The standard components of interface unit shown in block 292 are processor 305, storage 310, memory 308, transmitter/receiver 306, and a communication device such as network interface card or modem 312. In the preferred embodiment these components are embedded in the external stimulator 42 and can also be embedded in the programmer 85. These can be connected to the network 290 through appropriate security measures (Firewall) 293.

Another type of remote unit that may be accessed via central collaborative network 290 is remote computer 294. This remote computer 294 may be used by an appropriate attending physician to instruct or interact with interface unit 292, for example, instructing interface unit 292 to send instruction downloaded from central computer 286 to remote implanted unit.

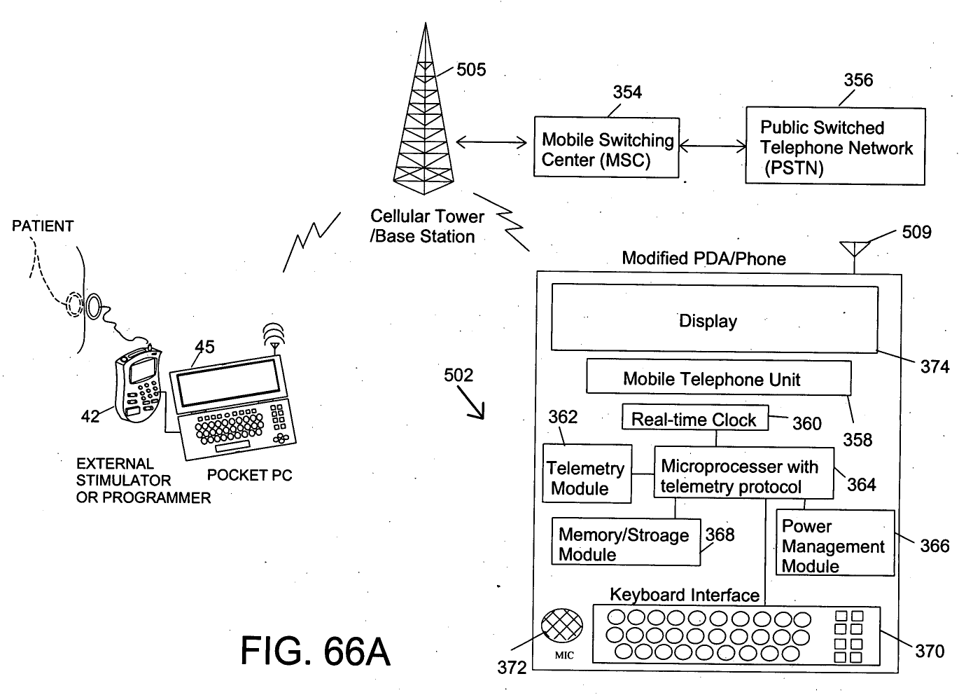

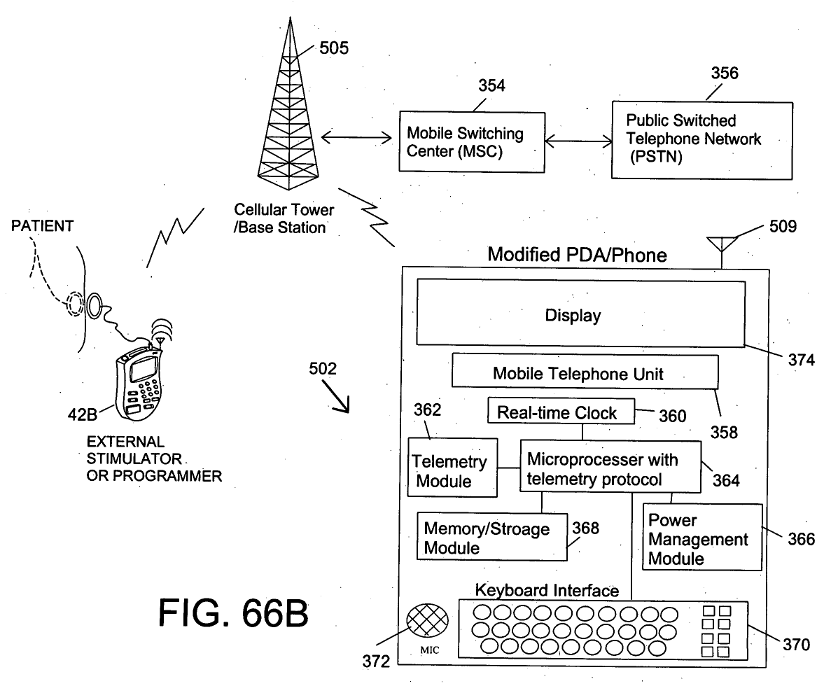

Shown in conjunction with FIGS. 66A and 66B the physician's remote communication's module is a Modified PDA/Phone 502 in this embodiment. The Modified PDA/Phone 502 is a microprocessor based device as shown in a simplified block diagram in FIGS. 76A and 76B. The PDA/Phone 502 is configured to accept PCM/CIA cards specially

configured to fulfill the role of communication module 292 of the present invention. The Modified PDA/Phone 502 may operate under any of the useful software including Microsoft Window's based, Linux, Palm OS, Java OS, SYMBIAN, or the like.

The telemetry module 362 comprises an RF telemetry antenna 142 coupled to a telemetry transceiver and antenna driver circuit board which includes a telemetry transmitter and telemetry receiver. The telemetry transmitter and receiver are coupled to control circuitry and registers, operated under the control of microprocessor 364. Similarly, within stimulator a telemetry antenna 142 is coupled to a telemetry transceiver comprising RF telemetry transmitter and receiver circuit. This circuit is coupled to control circuitry and registers operated under the control of microcomputer circuit.

With reference to the telecommunications aspects of the invention, the communication and data exchange between Modified PDA/Phone 502 and external stimulator 42 operates on commercially available frequency bands. The 2.4-to-2.4853 GHz bands or 5.15 and 5.825 GHz, are the two unlicensed areas of the spectrum, and set aside for industrial, scientific, and medical (ISM) uses. Most of the technology today including this invention, use either the 2.4 or 5 GHz radio bands and spread-spectrum technology.

The telecommunications technology, especially the wireless internet technology, which this invention utilizes in one embodiment, is constantly improving and evolving at a rapid pace, due to advances in RF and chip technology as well as software development. Therefore, one of the intents of this invention is to utilize “state of the art” technology available for data communication between Modified PDA/Phone 502 and external stimulator 42. The intent of this invention is to use 3G technology for wireless communication and data exchange, even though in some cases 2.5G is being used currently.

For the system of the current invention, the use of any of the “3G” technologies for communication for the Modified PDA/Phone 502, is considered within the scope of the invention. Further, it will be evident to one of ordinary skill in the art that as future 4G systems, which will include new technologies such as improved modulation and smart antennas, can be easily incorporated into the system and method of current invention, and are also considered within the scope of the invention.

The present invention may be embodied in other specific forms without departing from the spirit or essential attributes thereof. It is therefore desired that the present embodiment be considered in all aspects as illustrative and not restrictive, reference being made to the appended claims rather than to the foregoing description to indicate the scope of the invention.

What is claimed is:

1. A method of providing rectangular and/or complex electrical pulses to sacral nerve(s) and/or its branches or parts thereof of a patient, for treating or alleviating the symptoms for at least one of urinary incontinence, fecal incontinence, urological disorders, comprising the steps of:
providing an implanted pulse generator, capable of generating rectangular and/or complex electrical pulses, wherein said implanted pulse generator comprises microprocessor, circuitry, memory, and power source;
providing at least one predetermined/pre-packaged program(s) of said neuromodulation therapy stored in memory of said implantable pulse generator, wherein said predetermined/pre-packaged program(s) define neuromodulation parameters of pulse amplitude, pulse-width, pulse frequency, on-time and off-time;
providing at least one implanted lead(s) in electrical contact with said implanted pulse generator, wherein said implanted lead(s) comprising at least one electrode adapted to be in contact with said sacral nerve(s) or branches;
providing programmer means for activating and/or programming said implanted pulse generator, wherein bi-directional inductive telemetry is used to exchange data with said implanted pulse generator; and
selectively choosing between said at least one predetermined/pre-packaged program and activating said selected program.

2. The method of claim 1, wherein said urological disorders comprises overflow incontinence, stress incontinence, idiopathic chronic urinary retention, interstitial cystitis, neuro-urological disorder, vesico-urethral dysfunctions, bladder inflammation, bladder pain, pelvic pain, constipation, and genito-urinary disorders such as prostatitis, prostatalgia, and prostatodynia.

3. The method of claim 1, wherein said sacral nerve(s) and/or its branches or parts thereof comprises at least one of sacral nerves S₁, S₂, S₃, S₄, pudendal nerve, superior gluteal nerve, lumbo-sacral trunk, inferior gluteal nerve, common fibular nerve, tibial nerve, posterior femoral cutaneous nerve, sciatic nerve, and obturator nerve.

4. The method of claim 1, wherein said electrical pulses to said sacral nerve(s) and/or its branches may be provided unilaterally or bilaterally.

5. The method of claim 1, wherein said at least one predetermined/pre-packaged program(s) can be modified with an external programmer.

6. The method of claim 1, wherein said implanted pulse generator further comprises a power source which is rechargeable.

7. The method of claim 6, wherein said rechargeable power source in said implanted pulse generator is recharged with an external system, via inductively coupled energy transfer.

8. The method of claim 1, wherein said implanted pulse generator further comprises circuitry switchable between inductively coupled energy transfer, and telemetry for said implanted pulse generator.

9. The method of claim 1, wherein said implanted pulse generator further comprises telemetry means for remote device interrogation and/or programming over a wide area network.

10. A method of neuromodulating sacral nerve(s) and/or its branches or parts thereof for treating or alleviating the symptoms for at least one of fecal incontinence, urinary incontinence including overflow incontinence and urinary stress incontinence, idiopathic chronic urinary retention, interstitial cystitis, neuro-urological disorder, vesico-urethral dysfunctions, bladder inflammation, bladder pain, pelvic pain, constipation, and genito-urinary disorders such as prostatitis, prostatalgia, and prostatodynia, with rectangular and/or complex electric pulses, comprising the steps of:
providing an implanted pulse generator to supply said rectangular and/or complex electric pulses, wherein said implanted pulse generator is one from a group comprising: a combination implantable device, wherein said implantable device comprises both a stimulus-receiver module and a programmable implanted pulse generator (IPG) module; an implantable pulse generator (IPG) comprising a rechargeable battery; or a programmable implanted pulse generator (IPG);
providing at least one predetermined/pre-packaged program(s) stored in memory to control the output of said implanted pulse generator, wherein said predetermined/pre-packaged program(s) defines neuromodulation parameters of pulse amplitude, pulse-width, pulse frequency, on-time and off-time;
providing at least one implanted lead in electrical contact with said implanted pulse generator, and comprising at least one electrode adapted to be in contact with said sacral nerve(s) or its branches;
providing means for activating and/or programming said implantable pulse generator, wherein bi-directional inductive telemetry is used to exchange data with said implanted pulse generator; and
activating said at least one predetermined/pre-packaged program to emit said rectangular and/or complex electric pulses to said sacral nerve(s) and/or its branches,
whereby, neuromodulation of said sacral nerve(s) and/or its branches is provided according to said at least one predetermined/pre-packaged program.

11. The method of claim 10, wherein said implanted pulse generator further comprises telemetry means for remote device interrogation and/or programming over a wide area network.

12. The method of claim 10, wherein said electrical pulses to said sacral nerve(s) and/or its branches may be provided unilaterally or bilaterally.

13. A system for providing rectangular and/or complex electrical pulses to sacral nerve(s) and/or its branches or parts thereof, for treating or alleviating the symptoms for at least one of urinary incontinence, fecal incontinence, urological disorders, comprising:
an implantable pulse generator capable of generating rectangular and/or complex electrical pulses, comprising microprocessor, circuitry, memory, and power source;
at least one predetermined/pre-packaged program(s) of neuromodulation therapy stored in memory of said implantable pulse generator to control electrical pulses emitted by the implantable pulse generator, wherein said predetermined/pre-packaged program(s) define neuromodulation parameters of pulse amplitude, pulse-width, pulse frequency, on-time and off-time;
an implantable lead in electrical contact with said implantable pulse generator, wherein said implantable lead comprising at least one electrode adapted to be in contact with said sacral nerve(s) or branches; and
an external programmer means for activating and/or programming said implantable pulse generator, wherein bidirectional inductive telemetry is used to exchange data with said implantable pulse generator.

14. The system of claim 13, wherein said sacral nerve(s) and/or its branches or parts thereof comprises at least one of sacral nerves S₁, S₂, S₃, S₄, pudendal nerve, superior gluteal nerve, lumbo-sacral trunk, inferior gluteal nerve, common fibular nerve, tibial nerve, posterior femoral cutaneous nerve, sciatic nerve, and obturator nerve.

15. The system of claim 13, wherein said urological disorders comprises at least one of overflow incontinence, stress incontinence, idiopathic chronic urinary retention, interstitial cystitis, neuro-urological disorder, vesico-urethral dysfunctions, bladder inflammation, bladder pain, pelvic pain, constipation, and genito-urinary disorders such as prostatitis, prostatalgia, and prostatodynia.

16. The system of claim 13, wherein said at least one predetermined/pre-packaged program(s) can be modified with an external programmer.

17. The system of claim 13, wherein said implanted pulse generator further comprises a rechargeable power source which is recharged with an external system, via inductively coupled energy transfer.

18. The system of claim 13, wherein said implanted pulse generator further comprises circuitry switchable between inductively coupled energy transfer, and telemetry for said implanted pulse generator.

19. The system of claim 13, wherein external components of said implantable pulse generator system further comprises telemetry means for remote device interrogation and/or programming over a wide area network.

20. The system of claim 13, wherein said implanted lead comprises a lead body with insulation which is one from the group consisting of polyurethane, silicone, and silicone with polytetrafluoroethylene (PTFE).

21. The system of claim 13, wherein said at least one electrode of said implanted lead comprises a material selected from the group consisting of platinum, platinum/iridium alloy, platinum/iridium alloy coated with titanium nitride, and carbon.
